(19) United States
(12) Patent Application Publication
(10) Pub. No.: US 2008/0119549 A1
(43) Pub. Date: May 22, 2008

(54) METABOLICALLY-STABILIZED INHIBITORS OF FATTY ACID AMIDE HYDROLASE

(75) Inventors: Olivier DASSE, Foothill Ranch, CA (US); David Putman, Irvine, CA (US); Timothy R. Compton, Issaquah, WA (US); Jeff A. Parrott, Irvine, CA (US)

Correspondence Address: ORGANON USA, INC.;PATENT DEPARTMENT, 56 LIVINGSTON AVENUE, ROSELAND, NJ, 07068, US

(73) Assignee: N.V. Organon, Oss (NL)

(21) Appl. No.: 11/773,675

(22) Filed: Jul. 5, 2007

Related U.S. Application Data
(60) Provisional application No. 60/866,568, filed on Nov. 20, 2006.

(51) Int. Cl. A61K 31/27; A61P 29/00; C07C 271/10

(52) U.S. Cl. 514/488; 560/24

(57) ABSTRACT

Pharmacological inhibition of fatty acid amide hydrolase (FAAH) activity leads to increased levels of fatty acid amides. Esters of alkylcarbamic acids are disclosed that are inhibitors of FAAH activity. Compounds disclosed herein inhibit FAAH activity. Described herein are processes for the preparation of esters of alkylcarbamic acid compounds, compositions that include them, and methods of use thereof.

### CROSS-REFERENCE TO RELATED APPLICATIONS

This application claims the benefit of U.S. Provisional Application No. 60/866,568, entitled “METABOLICALLY-STABILIZED INHIBITORS OF FATTY ACID AMIDE HYDROLASE,” filed Nov. 20, 2006, which is incorporated by reference in its entirety.

### FIELD OF THE INVENTION

Described herein are compounds, methods of making such compounds, pharmaceutical compositions and medicaments containing such compounds, and methods of using such compounds and compositions to inhibit the activity of fatty acid amide hydrolase (FAAH).

### BACKGROUND OF THE INVENTION

Fatty acid amide hydrolase (FAAH) is an enzyme that hydrolyzes the fatty acid amide (FAA) family of endogenous signaling lipids. General classes of FAAs include the N-acylethanolamines (NAEs) and fatty acid primary amides (FAPAs). Examples of NAEs include anandamide (AEA), palmitoylethanolamide (PEA) and oleoylethanolamide (OEA). Pharmacological inhibition of FAAH activity results in increases in the levels of these fatty acid amides.

### SUMMARY OF THE INVENTION

Compounds, compositions and methods for inhibiting the activity of fatty acid amide hydrolase (FAAH) are provided. Among the compounds provided herein are compounds that are inhibitors of fatty acid amide hydrolase (FAAH). Among the compounds provided are compounds that are metabolically-stabilized relative to the compound having the structure:

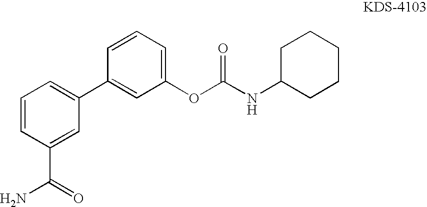

Processes for the preparation of such metabolically-stabilized compounds that inhibit the activity of fatty acid amide hydrolase, compositions that include the compounds, as well as methods of use thereof are also provided.

Such metabolic stabilization includes improved pharmacokinetic and pharmacodynamic parameters, including an increased bioavailability, an increased half-life, a decreased clearance rate, an increased Tₘₐₓ, an increased Cₘₐₓ, an increase area under the curve, or any combination of the foregoing.

Compounds provided herein include carbamates and ureas in which at least one of the groups covalently attached to the “N” portion of the carbamate or one of the two “N” portions of the urea is a (CH₂)_{z}(C₃-C₈ cycloalkyl), a (CH₂)_{z}(C₃-C₈ heterocycloalkyl), or a (CH₂)_{z}(C₇-C₁₂ polycycloalkyl) group, wherein z is 0 or 1, and wherein at least one of the carbon atoms in the cycloalkyl ring or, optionally, one of the carbon atoms in the polycycloalkyl ring, is monosubstituted or disubstituted, and wherein each substitution is independently selected from the group consisting of methyl, halogen, fluoromethyl or C₃-C₆ cycloalkyl; or wherein one carbon atom in the cycloalkyl ring is substituted with an oxo group; or wherein one carbon atom in the cycloalkyl ring is disubstituted to form a 3-, 4-, or 5-membered carbocyclic group; or wherein two adjacent atoms in the cycloalkyl ring are each substituted with a group that forms a 3-, 4-, 5-, or 6-membered carbocyclic group; or wherein at least one of the groups covalently attached to the “N” portion of the carbamate or one of the two “N” portions of the urea is an optionally substituted (CH₂)_{z}(bridged carbocylic group), z is 0 or 1, wherein the optional substitution is a methyl, halogen, or fluoromethyl group.

Compounds provided herein include carbamates and ureas in which at least one of the groups covalently attached to the “N” portion of the carbamate or one of the two “N” portions of the urea is selected from the group consisting of:

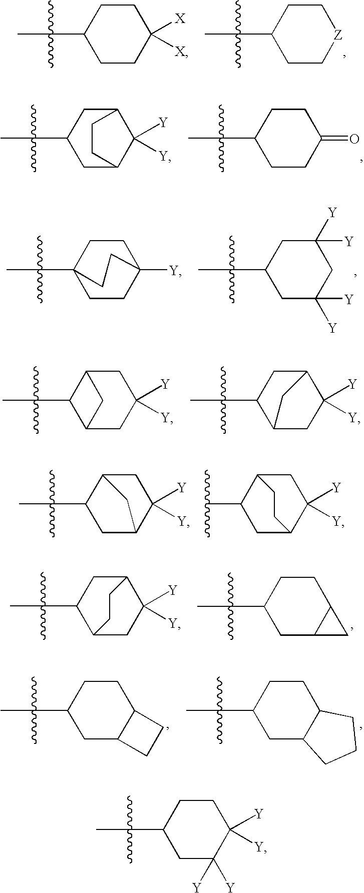

neopentyl, neohexyl, methylenecyclopropyl, methylenecyclobutyl, and methylenecyclopentyl; with the proviso that the groups attached to the “N” portion of the carbamate or the “N” portion of the urea is not an unsubstituted cyclohexyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; and
Z is O, N—(C₁-C₆ alkyl), or SO₂.

Compounds provided herein include those that have a structure of Formula (I) and pharmaceutically acceptable salts, N-oxides, solvates, esters, acids and prodrugs thereof. In certain embodiments, isomers and chemically protected forms of compounds having a structure represented by Formula (L) are also provided.

Provided herein are compounds of Formula (I):

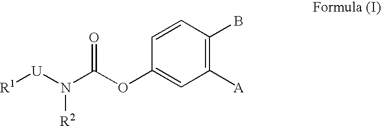

wherein:
R¹ is selected from the group consisting of:

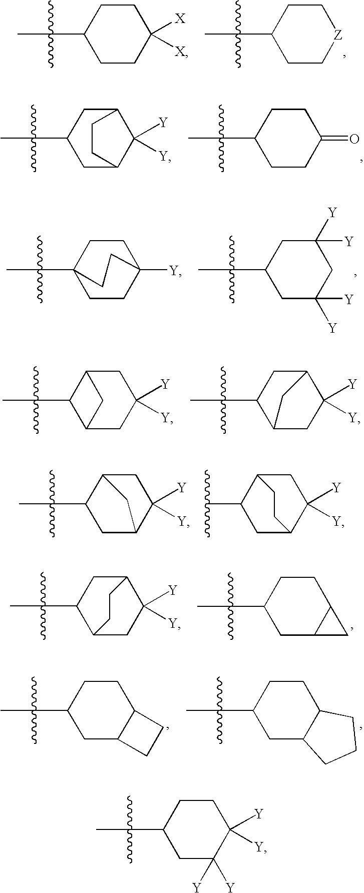

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted non-aromatic cyclic group comprising a C(O)—(CH₂)_{q}— moiety, wherein q is 1, 2, 3 or 4;
or A and B together form an optionally substituted heteroaromatic group comprising at least one N, NR², S, or group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, and an optionally substituted ketoheteroalkyl;
or one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and
pharmaceutically acceptable salts, pharmaceutically acceptable N-oxides, pharmaceutically active metabolites, pharmaceutically acceptable prodrugs, or pharmaceutically acceptable solvates thereof.

In one embodiment is a compound of Formula (I), wherein one of A or B is -L-G and the other is H.

In another embodiment is a compound of Formula (I), wherein A is -L-C.

In a further embodiment is a compound of Formula (I), wherein B is -L-G.

In yet a further embodiment is a compound of Formula (I), wherein R² is H.

In one embodiment is a compound of Formula (I), wherein:
L is a bond, or an optionally substituted group selected from among C₁-C₆ alkylene, C₁-C₆ ketoalkylene, —C(O)NR⁹—(CH₂)ₙ—, —NR⁹—C(O)—(CH₂)ₙ—.

In one embodiment, is a compound wherein:

L is a bond, or an optionally substituted group selected from among C₁-C₆ alkylene, —NR⁹—C(O)—(CH₂), —

In another embodiment is a compound of Formula (I), wherein L is —NR⁹—C(O)—(CH₂)ₙ—, G is H; R⁹ is H; and n is 1.

In yet another embodiment is a compound of Formula (I), wherein L is a bond.

In a further embodiment is a compound of Formula (I), wherein G is —CO₂H.

In yet a further embodiment is a compound of Formula (I), wherein L is CH₂; and G is —CO₂H.

In one embodiment is a compound of Formula (I), wherein G is tetrazolyl.

In another embodiment is a compound of Formula (I), wherein one of A or B is (CH₂)_{q}C(O)-alkyl, (CH₂)_{q}C(O)—N(R²)₂ and the other is H, alkyl, or heteroalkyl, and q is 0, 1, 2, 3, or 4.

In yet another embodiment is a compound of Formula (I), wherein one of A or B is (CH₂)_{q}C(O)—N(R²)₂; wherein q is 0, R² is H; and the other is H.

In a further embodiment is a compound of Formula (I), wherein one of A or B is (CH₂)_{q}C(O)—N(R²)₂; wherein q is 1, R² is H; and the other is H.

In yet a further embodiment is a compound of Formula (I), wherein A and B together form an optionally substituted heteroaromatic group comprising at least one N, NR², S, or O group.

In one embodiment is a compound of Formula (I), wherein A and B together form the optionally substituted heteroaromatic group comprising N and S.

In another embodiment is a compound of Formula (I), wherein A and B together form the optionally substituted heteroaromatic group comprising N and O.

In one embodiment is a compound of Formula (I), wherein the heteroaromatic group is optionally substituted with a CH₃ group.

In one aspect is a pharmaceutical composition comprising a compound of Formula (I), pharmaceutically acceptable salt, pharmaceutically acceptable N-oxide, pharmaceutically active metabolite, pharmaceutically acceptable prodrug, or pharmaceutically acceptable solvate and a pharmaceutically acceptable diluent, excipient or binder.

In one aspect is a method of treating pain in a patient comprising administering to the patient having pain a therapeutically effective amount of a compound, pharmaceutically acceptable salt, pharmaceutically acceptable N-oxide, pharmaceutically active metabolite, pharmaceutically acceptable prodrug, or pharmaceutically acceptable solvate of claim 1.

In one embodiment the pain is selected from the group consisting of nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In one embodiment, the compound is an irreversible inhibitor of fatty acid amide hydrolase.

In another embodiment, the compound does not substantially cross the blood-brain barrier.

In one aspect is a use of a compound of Formula (I) for inhibiting the activity of fatty acid amide hydrolase activity or for the treatment of a disease, disorder, or condition, that would benefit from inhibition of fatty acid amide hydrolase activity. In one embodiment, the disease, disorder or condition is pain. In a further embodiment the pain is selected from the group consisting of nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

Also provided herein are uses of a compound of claim of Formula (I) for the formulation of a medicament for the inhibition of fatty acid amide hydrolase (FAAH) and/or the treatment of pain.

Provided herein are articles of manufacture, comprising packaging material, a compound of Formula (I), which is effective for inhibiting the activity of fatty acid amide hydrolase (FAAH), within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of fatty acid amide hydrolase (FAAH).

Compounds provided herein include those that have a structure of Formula (II) and pharmaceutically acceptable salts, N-oxides, solvates, esters, acids and prodrugs thereof. In certain embodiments, isomers and chemically protected forms of compounds having a structure represented by Formula (II) are also provided.

In a further aspect are provided pharmaceutical compositions, which include a therapeutically effective amount of at least one of any of the compounds herein, or a pharmaceutically acceptable salt, pharmaceutically acceptable N-oxide, pharmaceutically active metabolite, pharmaceutically acceptable prodrug, or pharmaceutically acceptable solvate. In certain embodiments, the compositions provided herein further include a pharmaceutically acceptable diluent, excipient and/or binder.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by FAAH activity, or in which FAAH activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) one or more compounds provided herein.

In one aspect, provided herein are methods for treating a patient by administering a compound provided herein. In some embodiments, provided herein is a method of inhibiting the activity of fatty acid amide hydrolase or of treating a disease, disorder, or condition, which would benefit from inhibition of fatty acid amide hydrolase activity in a patient, which includes administering to the patient a therapeutically effective amount of at least one of any of the compounds herein, or pharmaceutically acceptable salt, pharmaceutically acceptable N-oxide, pharmaceutically active metabolite, pharmaceutically acceptable prodrug, or pharmaceutically acceptable solvate.

In certain embodiments, compounds and compositions provided herein are effective for the treatment, prevention, or amelioration of one or more symptoms of diseases, disorders or conditions that are selected from among acute or chronic pain, eating disorders, cardiovascular diseases, metabolic diseases, disorders or conditions, renal ischemia, cancers, disorders of the immune system, allergic diseases, parasitic, viral or bacterial infectious diseases, inflammatory diseases, osteoporosis, ocular conditions, pulmonary conditions, gastrointestinal diseases and urinary incontinence.

In other embodiments, compounds provided herein are effective for the treatment, prevention, or amelioration of one or more symptoms of diseases, disorders or conditions that are selected from among pain, nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In other embodiments, compounds provided herein are effective for the treatment, prevention, or amelioration of one or more symptoms of diseases, disorders or conditions that are selected from among Parkinson's disease, muscle spasticity, epilepsy, obesity, hyperlipidemia, insulin resistance syndrome, fatty liver disease, obesity, atherosclerosis, arteriosclerosis, metabolic disorders, feeding and fasting, alteration of appetite, hypertension, septic shock, cardiogenic shock, intestinal inflammation and motility, irritable bowel syndrome, colitis, diarrhea, ileitis, ischemia, cerebral ischemia, hepatic ischemia, myocardial infarction, arthritis, rheumatoid arthritis, spondylitis, shoulder tendonitis or bursitis, gouty arthritis, aolymyalgia rheumatica, thyroiditis, hepatitis, inflammatory bowel diseases, asthma, multiple sclerosis, chronic obstructive pulmonary disease (COPD), allergic rhinitis, and cardiovascular diseases.

Compounds provided herein are irreversible inhibitors of fatty acid amide hydrolase (FAAH). Compounds provided herein increase the levels of some endogenous fatty acid amides. Compounds provided herein increase the levels of endogenous fatty acid amides selected from among AEA, OEA and PEA.

In some embodiments, compounds provided herein do not substantially cross the blood-brain barrier, particularly compounds that are at least partially ionized (positively or negatively charged) at the pH of human serum. Fatty acid amide hydrolase (FAAH) is present throughout the body. In some cases, it is preferable to restrict FAAH inhibitors to peripheral tissues so as to minimize or eliminate any psychotropic effects. In some embodiments, compounds provided herein preferentially inhibit FAAH activity in peripheral tissues and fluids and minimize potentially undesired central nervous system side effects. In some embodiments, the incorporation of an ionizable group into a FAAH inhibitor compound decreases the ability of the compound to cross the blood-brain-barrier. In some embodiments, incorporation of an ionizable group into a FAAH inhibitor provides a FAAH inhibitor compound that preferentially inhibits FAAH activity in peripheral tissues. In some embodiments, the incorporation of an ionizable group into a FAAH inhibitor compound, such as, for example, compounds disclosed herein, may be used to inhibit FAAH activity in peripheral tissues in preference to CNS tissues. In some embodiments, the incorporation of an ionizable group into a FAAH inhibitor compound provides a FAAH inhibitor compound that does not substantially cross the blood-brain-barrier and is not an effective therapeutic in neural disorders.

In some embodiments, compounds provided herein are FAAH inhibitor compounds that are ionizable at physiological pH and do not substantially cross the blood brain barrier. Compounds that are ionizable at physiological pH are charged and do not substantially cross the blood brain barrier.

In some embodiments, compounds provided herein (if at least 10% ionized at the pH of human serum), after administration to a mammal, result in plasma AUC values that are at least 5 times greater than brain tissue AUC values, provided that the administration is conducted as described in Example 15. In other embodiments, compounds provided herein, after administration to a mammal, result in plasma AUC values that are at least 5 times, at least 6 times, at least 8 times, at least 10 times, at least 12 times, at least 14 times, at least 16 times, at least 18 times, at least 20 times, or at least 30 times greater than the brain tissue AUC values.

In some embodiments, compounds provided herein are administered to a human.

In some embodiments, compounds provided herein are orally administered.

In some embodiments, compounds provided herein are used for inhibiting fatty acid amide hydrolase (FAAH) activity. In some embodiments, compounds provided herein are used for inhibiting the activity of fatty acid amide hydrolase activity or for the treatment of a disease or condition that would benefit from inhibition of fatty acid amide hydrolase activity.

In other embodiments, compounds provided herein are used for the formulation of a medicament for the inhibition of fatty acid amide hydrolase (FAAH).

In certain embodiments, compounds and compositions provided herein are effective for the treatment, prevention, or amelioration of one or more symptoms of diseases, disorders or conditions that are selected from among acute or chronic pain, dizziness, vomiting, nausea, eating disorders, neurological and psychiatric pathologies, acute or chronic neurodegenerative diseases, epilepsy, sleep disorders, cardiovascular diseases, renal ischemia, cancers, disorders of the immune system, allergic diseases, parasitic, viral or bacterial infectious diseases, inflammatory diseases, osteoporosis, ocular conditions, pulmonary conditions, gastrointestinal diseases and urinary incontinence.

In other embodiments, compounds provided herein are effective for the treatment, prevention, or amelioration of one or more symptoms of diseases, disorders or conditions that are selected from among pain, nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

In other embodiments, compounds provided herein are effective for the treatment, prevention, or amelioration of one or more symptoms of diseases, disorders or conditions that are selected from among Parkinson's disease, muscle spasticity, epilepsy, obesity, hyperlipidemia, insulin resistance syndrome, fatty liver disease, obesity, atherosclerosis, arteriosclerosis, metabolic disorders, feeding and fasting, alteration of appetite, hypertension, septic shock, cardiogenic shock, intestinal inflammation and motility, irritable bowel syndrome, colitis, diarrhea, ileitis, ischemia, cerebral ischemia, hepatic ischemia, myocardial infarction, arthritis, rheumatoid arthritis, spondylitis, shoulder tendonitis or bursitis, gouty arthritis, aolymyalgia rheumatica, thyroiditis, hepatitis, inflammatory bowel diseases, asthma, multiple sclerosis, chronic obstructive pulmonary disease (COPD), allergic rhinitis, and cardiovascular diseases.

In certain other embodiments, compounds and compositions provided herein are effective for the treatment, prevention, or amelioration of one or more symptoms of pain and/or inflammation.

In one aspect, provided herein is a method of inhibiting fatty acid amide hydrolase activity in a mammal, which includes administering to the mammal a therapeutically effective amount of a compound or composition provided herein. In some embodiments the mammal is a human. In other embodiments, compound or composition is orally administered.

In another aspect, a compound provided herein is used for the formulation of a medicament for the inhibition of fatty acid amide hydrolase (FAAH).

Articles of manufacture containing packaging material, a compound or composition or pharmaceutically acceptable derivative thereof provided herein, which is effective for inhibiting the activity of fatty acid amide hydrolase (FAAH), within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of fatty acid amide hydrolase (FAAH), are provided.

Any of the combinations of the groups described above for the various variables is contemplated herein.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. All references cited herein, including patents, patent applications, and publications, are hereby incorporated by reference in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The novel features are set forth with particularity in the appended claims. A better understanding of the features and advantages of the present disclosure will be obtained by reference to the following detailed description that sets forth illustrative embodiments, in which the principles described herein are utilized.

Disclosed herein are compounds that inhibit the activity the activity of fatty acid amide hydrolase (FAAH), compositions that include the compounds, and methods of their use. Compounds disclosed herein are inhibitors of fatty acid amide hydrolase (FAAH) and are useful in the treatment of diseases, disorders, or conditions that would benefit from the inhibition of fatty acid amide hydrolase and increases in endogenous fatty acid amides.

### The Endocannabinoid System

The endocannabinoid signaling system is composed of three elements (Lambert et al. J. Med. Chem. 2005, vol. 48, no. 16, 5059-5087). The first is represented by the G protein-coupled receptors that bind endogenous and exogenous cannabinoid ligands. Two such receptors have been identified, the CB₁ receptor, which is found almost everywhere in the body, but is most abundant in the central nervous system (CNS) (Freund et al. Physiol. Rev. 2003; 83:1017-1066); and the CB₂ receptor, which is primarily expressed in immune cells and in hematopoietic cells, but is also present at low levels in the brain (Munro et al. Nature, 1993; 365:61-65; Van Sickle et al. Science 2005; 310:329-332; Hanus et al., Proc. Nat. Acad. Sci., U.S.A., 1999; 96:14228-14233).

The second element is represented by the endocannabinoids, naturally occurring lipid molecules that bind to and activate cannabinoid receptors (Devane et al. Science 1992; 258: 1946-1949; Mechoulam et al. Biochem. Pharmacol. 1995; 50:83-90; Sugria et al. Biochem. Biophys. Res. Commun. 1995; 215:89-97), are generated on demand by neurons and other cells (Di Marzo et al. Nature 1994; 372: 686-691; Giuffrida et al. Nat. Neurosci. 1999; 2:358-363; Stella et al. Nature 2001; 388:773-778), and are rapidly eliminated (Beltramo et al. FEBS Lett. 1997; 403:263-267; Stella et al. Nature 2001; 388:773-778).

The third element is represented by the proteins involved in the formation and elimination of the various endocannabinoid ligands (Piomelli, D. Nat. Rev. Neurosci. 2003; 4:873-884).

Cannabinoid receptors can be activated by endocannabinoids, as well as synthetic ligands.

Anandamide (arachidonoylethanolamide) was the first endocannabinoid substance to be discovered (Devane et al. Science 1992; 258:1946-1949; Piomelli, D. Nat. Rev. Neurosci. 2003; 4:873-884). Current evidence indicates that this lipid-derived mediator is released upon demand by stimulated neurons (Di Marzo et al. Nature, 1994; 372:686-691; Giuffrida et al. Nat. Neurosci. 1999, 2:358-363); activates cannabinoid receptors with high potency (Devane et al. Science 1992; 258:1946-1949), and is rapidly eliminated through a two-step process consisting of carrier-mediated internalization followed by intracellular hydrolysis (metabolism) (Beltramo et al. Science 1997; 277:1094-1097; Di Marzo et al. Nature 1994; 372:686-691; Hillard et al. J. Lipid Res. 1997; 38:2383-2398).

The endocannabinoids anandamide and 2-arachidonylglycerol (2-AG), both of which produce most of their effects by binding to the CB₁ receptor, have been shown to be tonically released and can control basal nociceptive thresholds (Meng et al., Nature 1998; September 24; 395(6700):381-3). In particular, anandamide acts as a CB₁ agonist and exhibits pharmacological activity in mice comparable to other synthetic cannabinoids.

### Fatty Acid Amide Hydrolase (FAAH)

Fatty acid amide hydrolase (FAAH) is an enzyme that hydrolyzes the fatty acid amide (FAA) family of endogenous signaling lipids. General classes of fatty acid amides include the N-acylethanolamines (NAEs) and fatty acid primary amides (FAPAs). Examples of NAEs include anandamide (AEA), palmitoylethanolamide (PEA) and oleoylethanolamide (OEA). An example of FAPAs includes 9-Z-octadecenamide or oleamide. (McKinney M K, Cravatt B F. 2005. Annu Rev Biochem 74:411-32)]. FAAH can act as a hydrolytic enzyme not only for fatty acid ethanolamides and primary amides, but also for esters, such as, for example, 2-arachidonylglycerol (2-AG) (Mechoulam et al. Biochem. Pharmacol. 1995; 50:83-90; Stella et al. Nature, 1997; 388:773-778; Suguria et al. Biochem. Biophys. Res. Commun. 1995; 215:89-97).

FAAH is abundantly expressed throughout the CNS (Freund et al. Physiol. Rev. 2003; 83:1017-1066) as well as in peripheral tissues, such as, for example, in the pancreas, brain, kidney, skeletal muscle, placenta, and liver (Giang, D. K. et al. Molecular Characterization of Human and Mouse Fatty Acid Amide Hydrolases. Proc. Natl. Acad. Sci. U.S.A. 1997, 94, 2238-2242; Cravatt et al. PNAS, 2004, vol. 101, no. 29, 10821-10826.).

Anandamide, or arachidonylethanolamide, is a NAE that acts as an endogenous ligand for the cannabinoid type 1 (CB₁) receptor (Devane W A, et al. 1992. Science 25 8:1946-49). Anandamide is rapidly eliminated through a two-step process consisting of carrier-mediated transport followed by intracellular hydrolysis by FAAH. The hydrolysis of anandamide by FAAH results in the formation of arachidonic acid and ethanolamine. The current postulated catalytic mechanism for hydrolysis of anandamide by FAAH involves nucleophilic attack of amino acid residue Serine 241 of FAAH on the amide moiety of anandamide, resulting in the formation of arachidonic acid and ethanolamine (Deutsch et al. The fatty acid amide hydrolase (FAAH) Prostaglandins, Leukotrienes and Essential Fatty Acids (2002) 66 (2&3), 201-210; Alexander et al. Chemistry & Biology, vol. 12, 1179-1187; 2005.).

Mutant mice lacking the gene encoding for FAAH display a profound reduction in hydrolysis activity for anandamide and other fatty acid amides and show signs of enhanced anandamide activity at cannabinoid receptors, leading to observable physiological phenomena such as reduced pain sensation (Cravatt B F, et al. 2001. Proc Nat Acad Sci USA 98: 9371-9376). This suggests that therapeutic agents that alter the activity of the FAAH enzyme can increase the actions of anandamide and other fatty acid amides in the body. Such agents may also avoid the multiple, often undesirable effects produced by indiscriminant activation of cannabinoid receptors by administration of Δ9-THC (the active ingredient in marijuana) and other direct-acting cannabinoids.

Many endogenous fatty acid amides, other than anandamide, do not bind the CB₁ receptor. Several of these lipids have been shown to produce specific cellular and behavioral effects, and may represent a large family of endogenous signaling lipids that act in vivo on receptor systems distinct from CB₁. These include palmitoylethanolamide (PEA) (Calignano A, et al. 1998. Nature 394:277-8 1; Jaggar S I, et al. 1998. Pain 76:189-99; Franklin A, Parmentier-Batteur et al. 2003. J Neurosci 23: 7767-75), stearoylethanolamide (SEA) (Terrazino et al. 2004 FASEB J: 18:1580-82; Maccarrone M, et al. 2002. Biochem J 366:137-44), and oleoylethanolamide (OEA) (deFonseca F R, et al. 2001. Nature 414:209-12; Fu J, et al. 2003. Nature 425:90-93; Fu J, et al. 2005. Neuropharmacology 48(8):1 147-53). Both OEA and PEA have been shown to activate peroxisome proliferator-activated receptor alpha (PPAR-alpha) (Fu J, et al. 2003. Nature 425:90-93; Guzman M, et al. 2004, J Biol Chem 279(27): 27849-54; Lo Verme J, et al. 2005. Cell Mol Life Sci 62(6): 708-16; Lo Verme J, et al. 2005. Life Sci 77(14): 1685-98; Lo Verme J, et al. 2005. Mol Pharmacol 67(1): 15-9). Through these actions, OEA and PEA can regulate several biological pathways including, but not limited to, feeding, metabolism, pain and inflammation. Therefore, agents that alter FAAH enzymatic activity can regulate the levels of a variety of fatty acid amides in vivo that, in turn, have therapeutic actions through a variety of targets.

Without being bound by theory, it is thought that certain fatty acid amides, such as, for example, OEA, act through the peroxisome proliferator-activated receptor α (PPAR-α) to regulate diverse physiological processes, including, e.g., feeding and lipolysis. Consistent with this, human adipose tissue has been shown to bind and metabolize endocannabinoids such as anandamide and 2-arachidonylglycerol. See Spoto et al., Aug. 22, 2006, Biochimie (E-publication ahead of print); and Matias et al. (2006), J. Clin. Endocrin. & Met., 91(8):3171-3180. Thus, inhibiting FAAH activity in vivo leads to reduced body fat, body weight, caloric intake, and liver triglyceride levels. However, unlike, other anti-lipidemic agents that act through PPAR-α, e.g., fibrates, FAAH inhibitors do not cause adverse side effects such as rash, fatigue, headache, erectile dysfunction, and, more rarely, anemia, leukopenia, angioedema, and hepatitis. See, e.g., Muscari et al. (2002), Cardiology, 97:115-121. An additional therapeutic property of FAAH inhibitors is that due to their ability to elevate anandamide levels, they effectively alleviate depression and anxiety, conditions often associated with energy metabolism disorders (EMDs) such as obesity. See Simon et al. (2006), Archives of Gen. Psychiatry, 63(7):824-830. In some embodiments, FAAH inhibitor compounds may be peripherally restricted and may not substantially affect neural disorders, such as, for example, depression and anxiety. Finally, agonism of cannabinoid receptors has also been shown to reduce the progression of atherosclerosis in animal models. See Steffens et al. (2005), Nature, 434:782-786; and Steffens et al. (2006), Curr. Opin. Lipid., 17:519-526. Thus, increasing the level of endogenous cannabinergic fatty acid amides (e.g., anandamide) is expected to effectively treat or reduce the risk of developing atherosclerosis.

Many fatty acid amides are produced on demand and rapidly degraded by FAAH. As a result, hydrolysis by FAAH is considered to be one of the essential steps in the regulation of fatty acid amide levels in the central nervous system as well as in peripheral tissues and fluids. The broad distribution of FAAH combined with the broad array of biological effects of fatty acid amides (both endocannabinoid and non-endocannabinoid mechanisms) suggests that inhibition of FAAH may lead to altered levels of fatty acid amides in many tissues and fluids and may be useful to treat many different conditions. FAAH inhibitors increase the levels of endogenous fatty acid amides. FAAH inhibitors block the degradation of endocannabinoids and increase the tissue levels of these endogenous substances. FAAH inhibitors can be used in this respect in the prevention and treatment of pathologies in which endogenous cannabinoids and or any other substrates metabolized by the FAAH enzyme are involved.

Inhibition of FAAH is expected to lead to an increase in the level of anadamide and other fatty acid amides. This increase in fatty acid amides may lead to an increase in the noiceptive threshold. Thus, in one embodiment, inhibitors of FAAH are useful in the treatment of pain. Such inhibitors might also be useful in the treatment of other disorders that can be treated using fatty acid amides or modulators of cannabinoid receptors, such as, for example, anxiety, eating disorders, metabolic disorders, cardiovascular disorders, and inflammation.

The various fatty acid ethanolamides have important and diverse physiological functions. As a result, inhibitor molecules that selectively inhibit FAAH enzymatic activity would allow a corresponding selective modulation of the cellular and extra-cellular concentrations of a FAAH substrate. FAAH inhibitors that are biologically compatible could be effective pharmaceutical compounds when formulated as therapeutic agents for any clinical indication where FAAH enzymatic inhibition is desired. In some embodiments, FAAH activity in peripheral tissues can be preferentially inhibited. In some embodiments, FAAH inhibitors that do substantially cross the blood-brain-barrier can be used to preferentially inhibit FAAH activity in peripheral tissues. In some embodiments, FAAH inhibitors that preferentially inhibit FAAH activity in peripheral tissues can minimize the effects of FAAH inhibition in the central nervous system. In some embodiments, it is preferred to inhibit FAAH activity in peripheral tissues and minimize FAAH inhibition in the central nervous system.

Diseases, disorders, syndromes and/or conditions, that would benefit from inhibition of FAAH enzymatic activity include, for example, Alzheimer's Disease, schizophrenia, depression, alcoholism, addiction, suicide, Parkinson's disease, Huntington's disease, stroke, emesis, miscarriage, embryo implantation, endotoxic shock, liver cirrhosis, atherosclerosis, cancer, traumatic head injury, glaucoma, and bone cement implantation syndrome.

Other diseases, disorders, syndromes and/or conditions that would benefit from inhibition of FAAH activity, include, for example, multiple sclerosis, retinitis, amyotrophic lateral sclerosis, immunodeficiency virus-induced encephalitis, attention-deficit hyperactivity disorder, pain, nociceptive pain, neuropathic pain, inflammatory pain, non-inflammatory pain, painful hemorrhagic cystitis, obesity, hyperlipidemia, metabolic disorders, feeding and fasting, alteration of appetite, stress, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, and inflammatory diseases.

Neurological and psychological disorders that would benefit from inhibition of FAAH activity include, for example, pain, depression, anxiety, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, diskenesia, seizure disorders, jet lag, and insomnia.

FAAH inhibitors can also be used in the treatment of a variety of metabolic syndromes, diseases, disorders and/or conditions, including but not limited to, insulin resistance syndrome, diabetes, hyperlipidemia, fatty liver disease, obesity, atherosclerosis and arteriosclerosis.

FAAH inhibitors are useful in the treatment of a variety of painful syndromes, diseases, disorders and/or conditions, including but not limited to those characterized by nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

Inhibition of FAAH activity can also be used in the treatment of a variety of conditions involving inflammation. These conditions include, but are not limited to arthritis (such as rheumatoid arthritis, shoulder tendonitis or bursitis, gouty arthritis, and aolymyalgia rheumatica), organ-specific inflammatory diseases (such as thyroiditis, hepatitis, inflammatory bowel diseases), asthma, other autoimmune diseases (such as multiple sclerosis), chronic obstructive pulmonary disease (COPD), allergic rhinitis, and cardiovascular diseases.

FAAH inhibitors may also be useful in the treatment of other disorders such as loss of appetite, respiratory disorders, allergies, and traumatic brain injury.

In some cases, FAAH inhibitors are useful in preventing neurodegeneration or for neuroprotection.

In addition, it has been shown that when FAAH activity is reduced or absent, one of its substrates, anandamide, acts as a substrate for COX-2, which converts anandamide to prostamides (Weber et al. J Lipid. Res. 2004; 45:757). Concentrations of certain prostamides may be elevated in the presence of a FAAH inhibitor. Certain prostamides are associated with reduced intraocular pressure and ocular hypotensivity. Thus, in one embodiment, FAAH inhibitors may be useful for treating glaucoma.

In some embodiments, FAAH inhibitors can be used to treat or reduce the risk of EMDs, which include, but are not limited to, obesity, appetite disorders, overweight, cellulite, Type I and Type II diabetes, hyperglycemia, dyslipidemia, steatohepatitis, liver steatosis, non-alcoholic steatohepatitis, Syndrome X, insulin resistance, diabetic dyslipidemia, anorexia, bulimia, anorexia nervosa, hyperlipidemia, hypertriglyceridemia, atherosclerosis, arteriosclerosis, inflammatory disorders or conditions, Alzheimer's disease, Crohn's disease, vascular inflammation, inflammatory bowel disorders, rheumatoid arthritis, asthma, thrombosis, or cachexia.

In other embodiments, FAAH inhibitors can be used to treat or reduce the risk of insulin resistance syndrome and diabetes, i.e., both primary essential diabetes such as Type I Diabetes or Type II Diabetes and secondary nonessential diabetes. Administering a composition containing a therapeutically effective amount of an in vivo FAAH inhibitor reduces the severity of a symptom of diabetes or the risk of developing a symptom of diabetes, such as atherosclerosis, hypertension, hyperlipidemia, liver steatosis, nephropathy, neuropathy, retinopathy, foot ulceration, or cataracts.

In another embodiment, FAAH inhibitors can be used to treat food abuse behaviors, especially those liable to cause excess weight, e.g., bulimia, appetite for sugars or fats, and non-insulin-dependent diabetes.

In some embodiments, FAAH inhibitors can be used to treat a subject suffering from an EMD and also suffers from a depressive disorder or from an anxiety disorder. Preferably, the subject is diagnosed as suffering from the depressive or psychiatric disorder prior to administration of the FAAH inhibitor composition. Thus, a dose of a FAAH inhibitor that is therapeutically effective for both the EMD and the depressive or anxiety disorder is administered to the subject. Methods for treatment of anxiety and depressive disorders by FAAH inhibition are described in, e.g., U.S. patent application Ser. Nos. 10/681,858 and 60/755,035.

Preferably, the subject to be treated is human. However, the methods can also be used to treat non-human mammals. Animal models of EMDs such as those described in, e.g., U.S. Pat. No. 6,946,491 are particularly useful.

Symptoms, diagnostic tests, and prognostic tests for each of the above-mentioned conditions are known in the art. See, e.g., “Harrison's Principles of Internal Medicine©,” 16th ed., 2004, The McGraw-Hill Companies, Inc., and the “Diagnostic and Statistical Manual of Mental Disorders©,” 4th ed., 1994, American Psychiatric Association.

FAAH inhibitor compositions can also be used to decrease body-weight in individuals wishing to decrease their body weight for cosmetic, but not necessarily medical considerations.

A FAAH inhibitor composition can be administered in combination with a drug for lowering circulating cholesterol levels (e.g., statins, niacin, fibric acid derivatives, or bile acid binding resins). FAAH inhibitor compositions can also be used in combination with a weight loss drug, e.g., orlistat or an appetite suppressant such as diethylpropion, mazindole, orlistat, phendimetrazine, phentermine, or sibutramine.

The methods described herein can also include providing an exercise regimen or providing a calorie-restricted diet (e.g., a triglyceride-restricted diet) to the subject.

Esters of alkylcarbamic acids and alkylthiocarbamic acids have shown promise as selective FAAH inhibitors (Kathuria et al., Nat. Med. 2003, 9:76-81). A series of alkylcarbamic acid aryl esters, such as, for example, cyclohexylcarbamic acid 3′-carbamoylbiphenyl-3-yl ester (also known as 5′-carbamoylbiphenyl-3-yl cyclohexyl carbamate, UCM597, URB597, and KDS-4103 (URB-597)), have been shown to be potent and selective inhibitors of FAAH activity. Alkylcarbamic acid aryl esters, such as, for example, cyclohexylcarbamic acid 3′-carbamoylbiphenyl-3-yl ester, have been shown to be potent and selective inhibitors of FAAH activity, which do not significantly interact with selected serine hydrolases or with cannabinoid receptors (Mor et al. J. Med. Chem. 2004, 47:4998-5008; Piomelli et al. International Patent Publication No. WO 2004/033422; incorporated by reference).

Alkylcarbamic acid aryl esters inhibit FAAH activity through an irreversible interaction with FAAH, possibly due to a nucleophilic attack of an active serine residue (Serine 241) of FAAH on the carbamate moiety of the alkylcarbamic acid aryl ester compounds (Kathuria et al. Nature Medicine, vol. 9, no. 1, 76-81, 2003; Deutsch et al. Prostaglandins, Leukotrienes and Essential Fatty Acids (2002) 66(2&3), 201-210; Alexander et al. Chemistry & Biology, vol. 12, 1179-1187; 2005.). Metabolism of the alkylcarbamic acid aryl ester inhibitors by the FAAH enzyme results in the hydrolysis of the carbamate compounds and release of the aryloxy portion of the alkylcarbamic acid aryl ester inhibitor.

Provided herein are compound, which are esters of alkylcarbamic acids, compositions that include them, and methods of their use. Compounds provided herein have a structure selected from among:

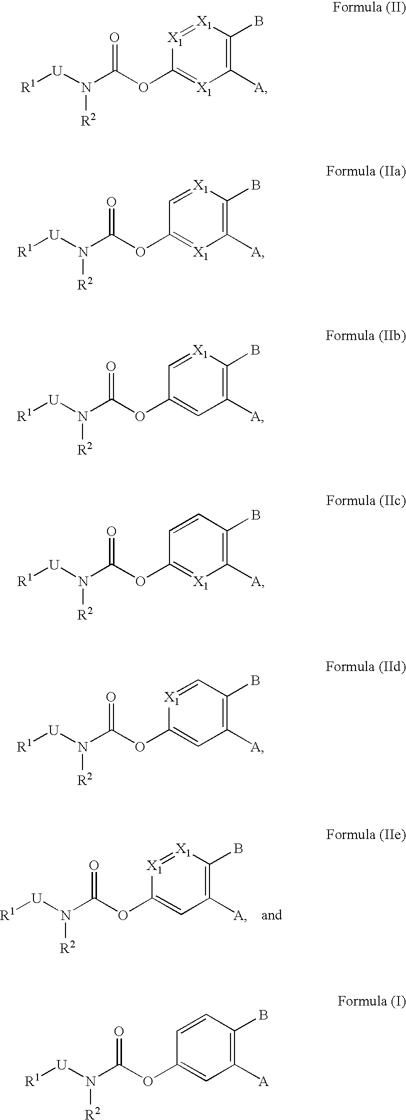

wherein:
R¹ is selected from the group consisting of:

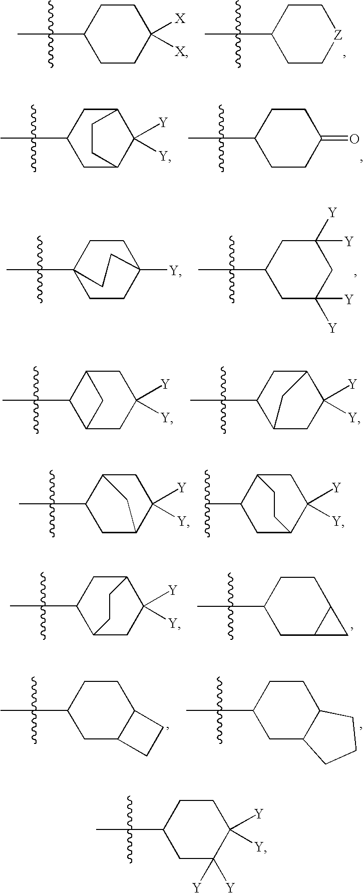

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
one of A or B is (CH₂)_{q}C(O)-alkyl, (CH₂)_{q}C(O)—N(R²)₂ and the other is H, alkyl, or heteroalkyl, q is 0, 1, 2, 3, or 4;
or A and B together form an optionally substituted non-aromatic cyclic group comprising a C(O)—(CH₂)_{q}-moiety, wherein q is 1, 2, 3 or 4;
or A and B together form an optionally substituted heteroaromatic group comprising at least one N, NR², S, or group;
or A and B together form an optionally substituted non-aromatic or aromatic carbocycle group;
or A and B together form an optionally substituted oxo-substituted heterocycle;
or A and B are each independently selected from among H, an optionally substituted alkyl, an optionally substituted heteroalkyl, an optionally substituted heterocyclic group, an optionally substituted aryl group, an optionally substituted heteroaryl group, an optionally substituted ketoalkyl, and an optionally substituted ketoheteroalkyl;
one of A or B is -L-G and the other is selected from among H and an optionally substituted C₁-C₆ alkyl; or
A and B are both selected from among H and an optionally substituted C₁-C₆ alkyl provided that at least one X₁ is present and is N;
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(—NR¹⁰)N(R⁹)—(CH₂)ₙ—, and —NR⁹C(═NR¹⁰)N(R⁹)—(CH₂)ₙ—;
G is tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₓOH, —OP(═O)OR⁸OH, —OP(═O)R¹⁰H, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
X₁ is independently CH or N, provided that at least one X₁ is N;
n is 1, 2, 3, or 4; x is 1, 2, or 3; y is 0, 1, or 2; and
   pharmaceutically acceptable salts, pharmaceutically acceptable N-oxides, pharmaceutically active metabolites, pharmaceutically acceptable prodrugs, or pharmaceutically acceptable solvates thereof.

Further compounds provided herein have a structure selected from among:

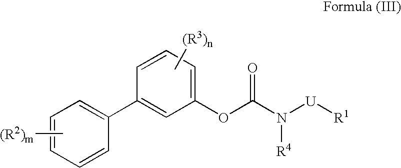

wherein:
R¹ is selected from the group consisting of:

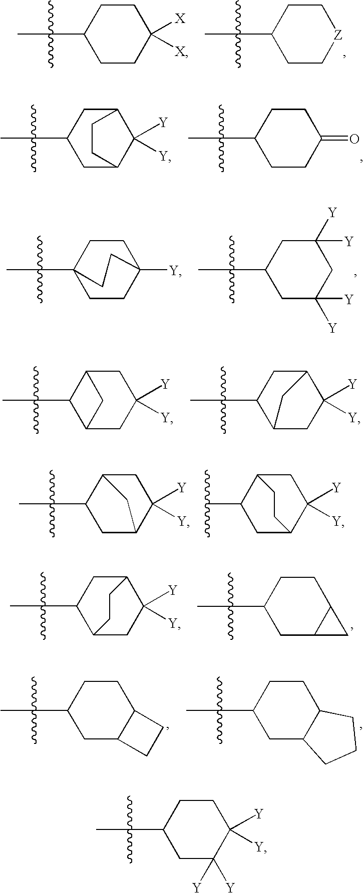

neopentyl, neohexyl, methylenecyclopropyl, methylenecyclobutyl, and methylenecyclopentyl; with the proviso that R¹ is not unsubstituted cyclohexyl;
R⁴ is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), arloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl; and m and n are each independently 0-3; and
pharmaceutically acceptable salts, pharmaceutically acceptable N-oxides, pharmaceutically active metabolites, pharmaceutically acceptable prodrugs, or pharmaceutically acceptable solvates thereof.

In some embodiments, the compound of Formula (III) has the structure:

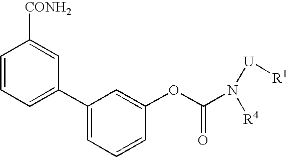

wherein:
R¹ is selected from the group consisting of:

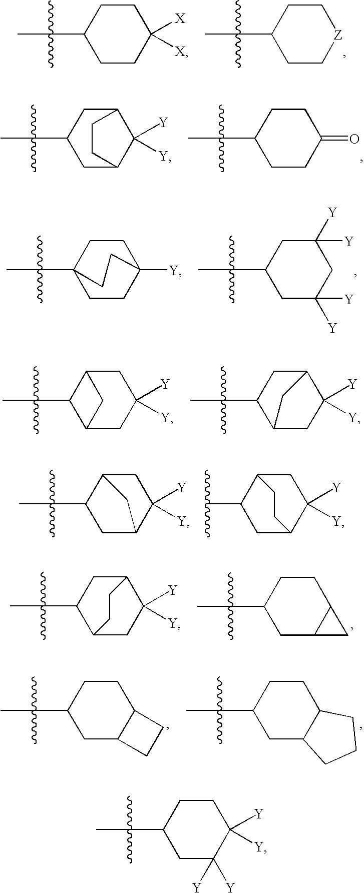

neopentyl, neohexyl, methylenecyclopropyl, methylenecyclobutyl, and methylenecyclopentyl, with the proviso that R¹ is not unsubstituted cyclohexyl;
R⁴ is H or an optionally substituted alkyl;
U is a bond or CH₂; and
pharmaceutically acceptable salts, pharmaceutically acceptable N-oxides, pharmaceutically active metabolites, pharmaceutically acceptable prodrugs, or pharmaceutically acceptable solvates thereof.

Further compounds described herein are:

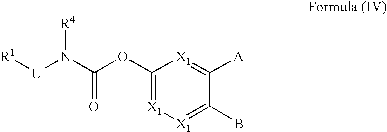

R¹ is selected from the group consisting of:

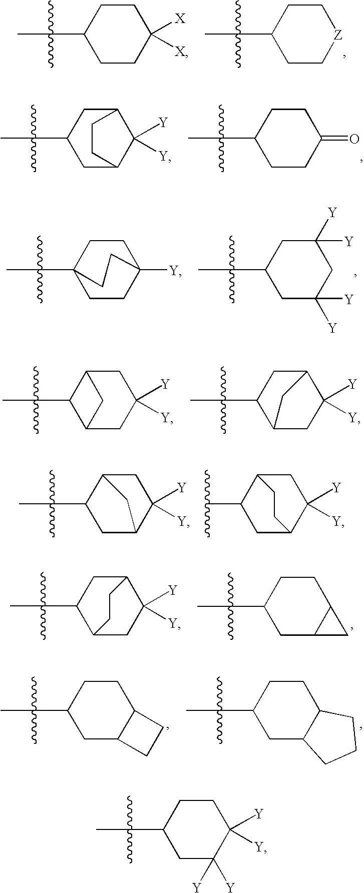

neopentyl, neohexyl, methylenecyclopropyl, methylenecyclobutyl, and methylenecyclopentyl; with the proviso that R¹ is not unsubstituted cyclohexyl;
R⁴ is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;

A and B are selected from:
(i) one of A or B is C(O)-alkyl or —C(O)N(alkyl)((CH₂)ₙG), and the other is H, alkyl, heteroalkyl;
   A and B can combine into a non-aromatic cyclic group;
   A and B can be substituted; or
(ii) A and B together form an optionally substituted heteroaromatic group; A and/or B are N, S, O, or CR⁹; or
(iii) A and B are H provided that at least one X₁ is not CH;

each X₁ is independently CH or N; and n is 1, 2, 3, or 4; and
pharmaceutically acceptable salts, pharmaceutically acceptable N-oxides, pharmaceutically active metabolites, pharmaceutically acceptable prodrugs, or pharmaceutically acceptable solvates thereof.

Further compounds described herein have the structure of Formula (1), Formula (2), Formula (3), Formula (4), Formula (5), Formula (6), Formula (7), Formula (8), Formula (9), Formula (10), Formula (11), Formula (12), Formula (13), Formula (14), Formula (15), Formula (16), Formula (17), Formula (18), Formula (19), Formula (20), Formula (21), Formula (22), Formula (23), Formula (24), Formula (25), Formula (26), Formula (27), Formula (28), or Formula (29) disclosed in U.S. Provisional Patent Application No. 60/755,035, filed on Dec. 29, 2005, herein incorporated by reference; provided that the R¹ group has the structure:

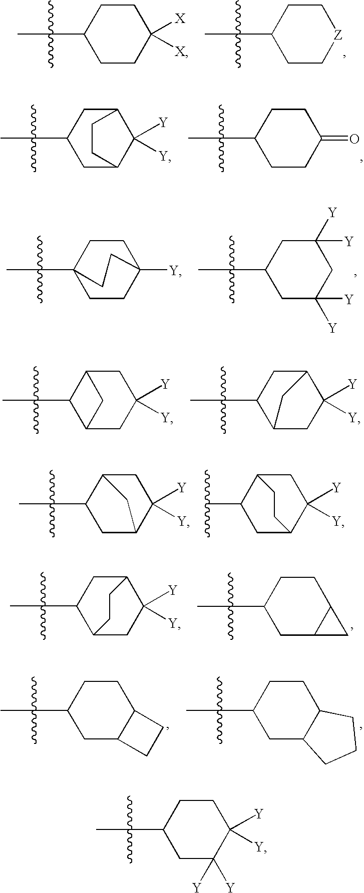

neopentyl, neohexyl, methylenecyclopropyl, methylenecyclobutyl, and methylenecyclopentyl; with the proviso that R¹ is not unsubstituted cyclohexyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl; and
Z is O, N—(C₁-C₆ alkyl), or SO₂; and
pharmaceutically acceptable salts, pharmaceutically acceptable N-oxides, pharmaceutically active metabolites, pharmaceutically acceptable prodrugs, or pharmaceutically acceptable solvates thereof.

### Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “Ito 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl moiety may be branched, straight chain, or cyclic (in which case, it would also be known as a “cycloalkenyl” group). Depending on the structure, an alkenyl group can be a monoradical or a diradical (i.e., an alkenylene group). Alkenyl groups can be optionally substituted.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡H, —C≡CCH₃ and —C≡CCH₂CH₃. The “R” portion of the alkynyl moiety may be branched, straight chain, or cyclic. Depending on the structure, an alkynyl group can be a monoradical or a diradical (i.e., an alkynylene group). Alkynyl groups can be optionally substituted.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “aromatic” refers to a planar ring having a delocalized n-electron system containing 4n+2π electrons, where n is an integer. Aromatic rings can be formed by five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “bond” or “single bond” refers to a chemical bond between two atoms, or two moieties when the atoms joined by the bond are considered to be part of larger substructure.

The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

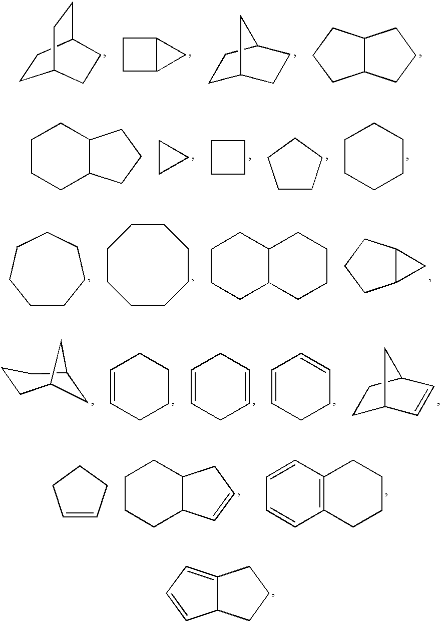

and the like. Depending on the structure, an cycloalkyl group can be a monoradical or a diradical (e.g., an cycloalkylene group).

As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

The term “halo” or, alternatively, “halogen” or “halide” means fluoro, chloro, bromo or iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the terms “heteroalkyl” “heteroalkenyl” and “heteroalkynyl” include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

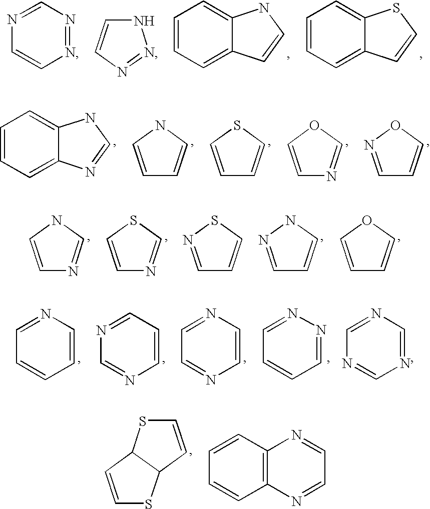

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

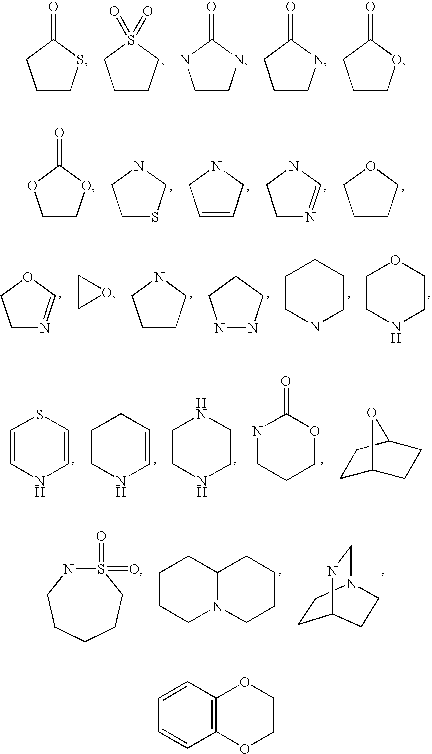

and the like. The term heteroalicyclic also includes all ring forms of the carbohydrates, including but not limited to the monosaccharides, the disaccharides and the oligosaccharides.

The term “monosaccharide” refers to any of several carbohydrates that cannot be broken down to simpler sugars via hydrolysis. By way of example only, monosaccharides include, trioses, such as, by way of example only, glyceraldehyde and dihydroxyacetone; tetroses, such as, by way of example only, erythrose, threose, and erythrulose; pentoses, such as, by way of example only, arabinose, lyxose, ribose, xylose, ribulose, and xylulose; hexoses, such as, by way of example only, allose, altrose, galactose, glucose, gulose, idose, mannose, talose, fructose, psicose, sorbose, and tagatose; heptoses, such as, by way of example only, mannoheptulose, sedoheptulose; octoses, such as, by way of example only, 2-keto-3-deoxy-manno-octonate; and nonoses, such as, by way of example only, sialose.

The term “dissacharide” refers to a carbohydrate composed of two monosaccharides. Examples of dissacharides, include, sucrose, lactose, maltose, trehalose, and cellobiose.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of the group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocyclic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The term “moiety” refers to a specific segment or functional group of a molecule. Chemical moieties are often recognized chemical entities embedded in or appended to a molecule.

As used herein, the term “cyano” refers to a group of formula —CN.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl. The protecting groups that may form the protective derivatives of the above substituents are known to those of skill in the art and may be found in references such as Greene and Wuts, above.

As used herein, the term “PEG” or “polyethyleneglycol” refers to a group of formula —CH₂—(O—CH₂—CH₂)_{q}—O-alkyl or —O—(CH₂—CH₂—O)_{q}-alkyl, —CH₂—(O—CH₂—CH₂)_{q}—OH, or —O(CH₂—CH₂—O)_{q}—H, where q is an integer between 1 and 300.

As used herein, the term “PPG” or “polypropyleneglycol” refers to a group of formula CH₂—(O—CHR_{M}—CHR_{M})_{q}—O-alkyl or —O—(CHR_{M}—CHR_{M}—O)_{q}-alkyl, —CH₂—(O—CHR_{M}—CHR_{M})_{q}—OH, or —O—(CHR_{M}—CHR_{M}—O)_{q}—H, where one of R_{M} is methyl and the other R_{M} is H, and q is an integer between 1 and 300.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof can be chosen by one skilled in the field to provide stable moieties and compounds.

### Compounds

Certain compounds that inhibit the activity of fatty acid amide hydrolase (FAAH) play a role in health. In certain embodiments, FAAH inhibitor compounds are useful in treating any of a variety of diseases, disorders or conditions. In certain embodiments, compounds provided herein are selective FAAH inhibitor compounds.

FAAH inhibitor compositions have been described in U.S. patent application Ser. Nos. 10/681,858, 60/755,035; U.S. Pat. Nos. 6,462,054, 6,949,574 and 6,891,043; International Patent Publication No. WO 04020430, WO 04067498, WO 04099176, WO 05033066, WO 02087569, WO 03065989, WO 9749667, WO 9926584, WO 04033652, and WO 06044617; Cravatt et al. Current Opinion in Chemical Biology, 2003, 7:469-475; Kathuria et al. Nature Medicine, vol. 9, no. 1, pp 76-81, 2003; Tarzia et al. J. Med. Chem. 2003, 46, 2352-2360; and Drysdale et al. Current Medicinal Chemistry, 2003, 10, 2719-2732.

Experiments have demonstrated that upon administration KDS-4103 is metabolized as follows:

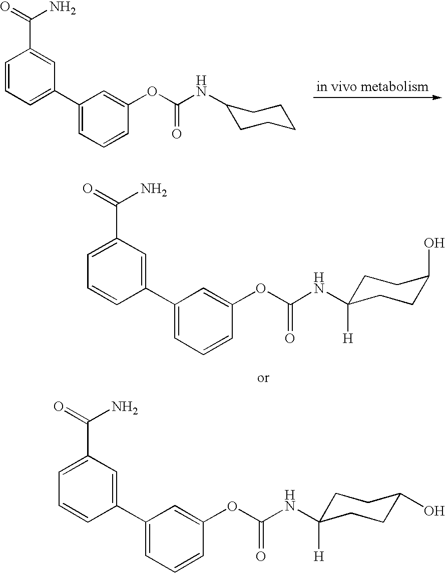

Based on this information, described herein are compounds that inhibit the activity of FAAH, but which are metabolically-stabilized relative to KDS-4103. Such stability can be conferred by replacing the cyclohexyl group of KDS-4103 with a group having the structure.

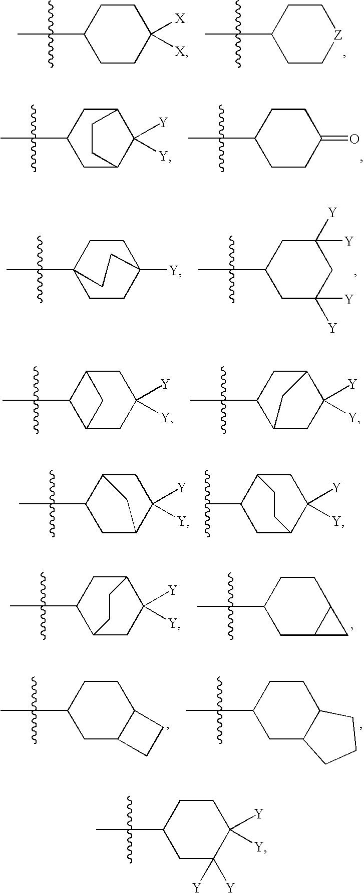

neopentyl, neohexyl, methylenecyclopropyl, methylenecyclobutyl, methylenecyclopentyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl; and
Z is O, N—(C₁-C₆ alkyl), or SO₂.

For example, in human S9 cells, the half-life of KDS-4103 is approximately 30 minutes. On the other hand:

### Human S9 ½ Life In-Vitro

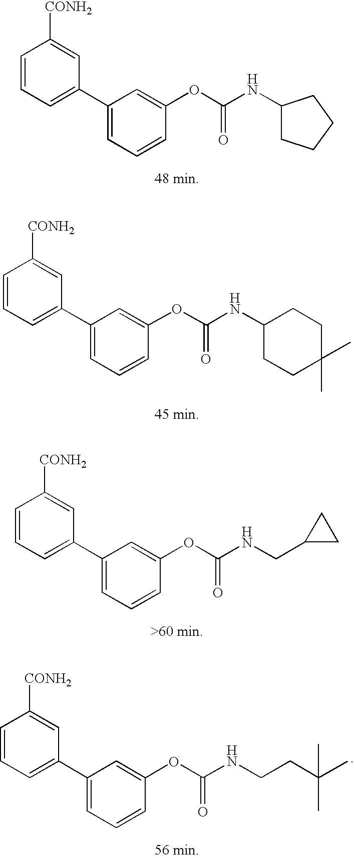

Further such a strategy of metabolically stabilizing a FAAH inhibitor can be employed with any of the FAAH inhibitors described herein, in particular compounds having the structure of Formula (I), Formula (II), Formula (III), or Formula (IV).

Also described herein are pharmaceutically acceptable salts, pharmaceutically active metabolites and pharmaceutically acceptable prodrugs of such compounds. Pharmaceutical compositions that include at least one such compound or a pharmaceutically acceptable salt, pharmaceutically active metabolite or pharmaceutically acceptable prodrug of such compound, are provided.

In some embodiments, compounds provided herein are ionizable and do not substantially cross the blood brain barrier. In some embodiments, provided herein are carbamate FAAH inhibitors that are ionizable at physiological pH, and therefore less likely to cross the blood brain barrier. In some embodiments, compounds provided herein have a moiety that is ionizable at physiological pH. In other embodiments, compounds provided herein have a charge at physiological pH. In some other embodiments, compounds provided herein are protonated at physiological pH. In other embodiments, compounds provided herein are deprotonated at physiological pH. Such FAAH inhibitors are particularly useful when it is desirable to minimize and/or avoid psychotropic effects caused by FAAH inhibition in the central nervous system.

In some embodiments, compounds provided herein have a structure selected from among:

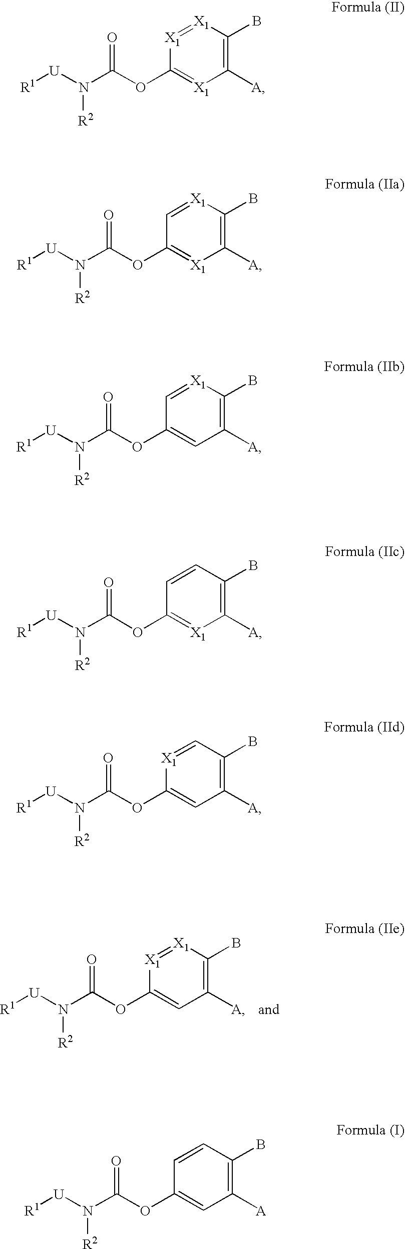

wherein:
R¹ is selected from the group consisting of:

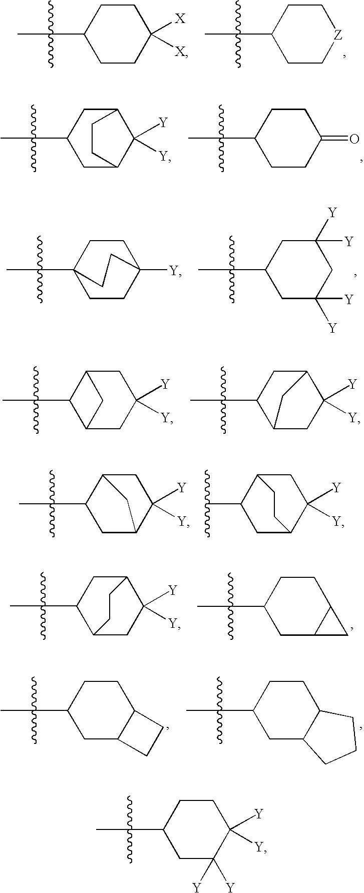

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
one of A or B is -L-G and the other is selected from among H and an optionally substituted C₁-C₆ alkyl; or
A and B are both H provided that at least one X₁ is present and is N;
L is a bond, or an optionally substituted group selected from among C₁-C₆ alkylene, C₁-C₆ heteroalkylene, C₁-C₆ ketoalkylene, a monosaccharide, a disaccharide, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;

G is H, tetrazolyl, —CH₂—(O—CH₂—CH₂), —O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R⁹)C(O)R⁸, —NR⁹C(O)N(R⁹)₂, —C(═NR¹³N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —C(R⁹)₂(OR⁹), —CO₂R⁹, —CON(R⁹)₂, —OS(═O)₂OR⁹, —OP(═O)₂OR⁹, or -L⁵-(substituted or unsubstituted heteroaryl containing 1-3 N atoms);
L⁵ is —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂—C(O)R⁸, —CN, and —NO₂;
X₁ is independently CH or N;
n is 1, 2, 3, or 4; and
   pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure according to Formula (II)

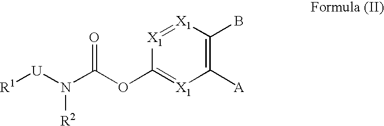

wherein:
R¹ is selected from the group consisting of:

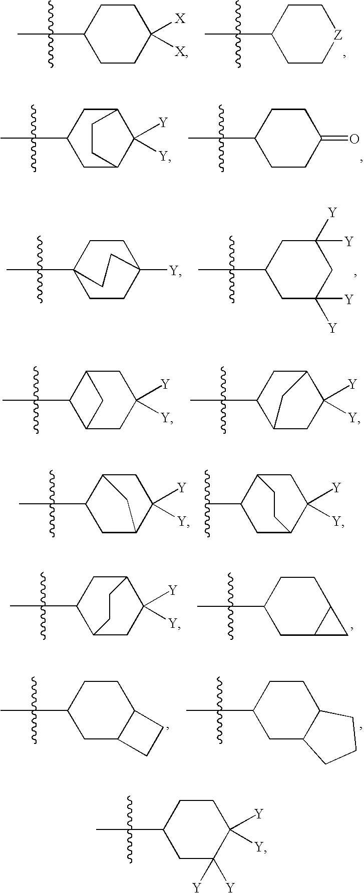

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
one of A or B is -L-G and the other is selected from among H and an optionally substituted C₁-C₆ alkyl, A and B can together form a ring structure; or
A and B are both H provided that at least one X₁ is N;
L is a bond, or an optionally substituted group selected from among C₁-C₆ alkylene, C₁-C₆heteroalkylene, C₁-C₆ ketoalkylene, a monosaccharide, a disaccharide, —C(O)NR⁹—(CH₂)ₙ—, —NR⁹—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R)C(O)R⁸, —NR⁹C(O)N(R⁹)₂, —C(═NR¹⁰)N(R)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —C(R⁹)₂(OR⁹), —CO₂R⁹, —CON(R)₂, —OS(═O)₂OR⁹, —OP(═O)₂OR⁹, or -L⁵-(substituted or unsubstituted heteroaryl containing 1-3 N atoms);
L⁵ is —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂—C(O)R¹, —CN, and —NO₂;
X₁ is independently CH or N;
n is 1, 2, 3, or 4; and
   pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure of Formula (IIa):

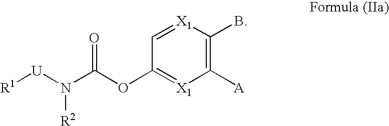

In other embodiments, compounds provided herein have a structure of Formula (IIb):

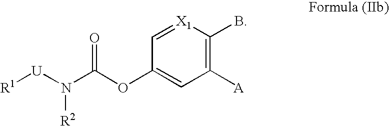

In some other embodiments, compounds provided herein have a structure of Formula (IIc):

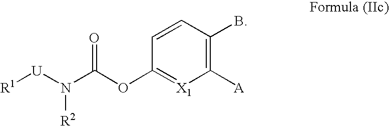

In another embodiment, compounds provided herein have a structure of Formula (IId):

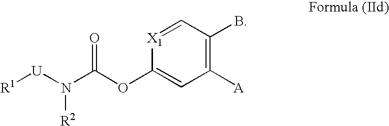

In yet some other embodiments, compounds provided herein have a structure of Formula (IIe):

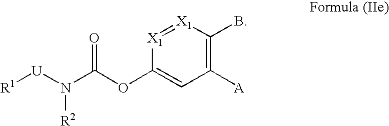

In some embodiments, both A and B are H provided that at least one X₁ is present and is N. In embodiments where X₁ is present, at least one X₁ is N.

In some embodiments, provided herein are compounds that have a structure of Formula (I):

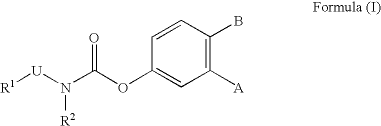

wherein:
R¹ is selected from the group consisting of:

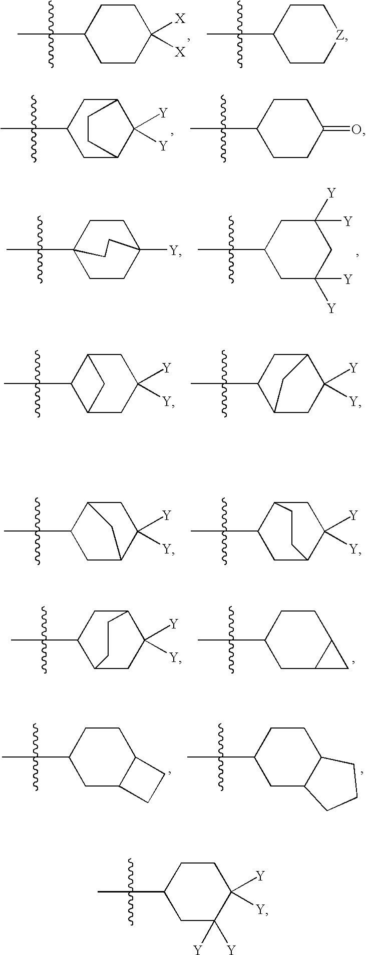

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, a monosaccharide, a disaccharide, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R⁹)C(O)R⁸, —NR⁹C(O)N(R⁹)₂, —C(═NR¹⁰)N(R⁹)₂, —NR⁹C(═NR¹⁰)N(R⁹)₂, —NR⁹C(═CHR¹⁰)N(R)₂, —C(O)NR⁹C(═NR¹⁰)N(R⁹)₂, —C(O)NR⁹C(═CHR¹⁰)N(R⁹)₂, —C(R⁹)₂(OR⁹), —CO₂R⁹, —CON(R⁹)₂, —OS(═O)₂OR⁹, —OP(═O)₂OR⁹, or -L⁵-(substituted or unsubstituted heteroaryl containing 1-3 N atoms);
L⁵ is —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂—C(O)R⁸, —CN, and —NO₂;
n is 1, 2, 3, or 4; and
   pharmaceutically acceptable salts, pharmaceutically acceptable N-oxides, pharmaceutically active metabolites, pharmaceutically acceptable prodrugs, or pharmaceutically acceptable solvates thereof.

For any and all of the embodiments, substituents can be selected from among a subset of the listed alternatives. For example, in some embodiments, one of A or B is -L-G and the other is H. In some embodiments, A is -L-G. In other embodiments, B is -L-G.

In some embodiments, R² is H.

In some embodiments, L is a bond, or an optionally substituted group selected from among C₁-C₆ alkylene, C₁-C₆ ketoalkylene, a monosaccharide, a disaccharide, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)—. In other embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, and —S(O)₂—(CH₂)ₙ—. In some other embodiments, L is a bond, or an optionally substituted group selected from among C₁-C₆ alkyl and C₁-C₆ ketoalkyl. In yet other embodiments, L is a bond.

In some embodiments, G is H, tetrazolyl, —CH₂—(O—CH₂—CH₂)_{q}—O—CH₃, —O—(CH₂—CH₂—O)_{q}—CH₃—CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NR⁹S(═O)₂R⁸, —S(═O)₂N(R⁹)₂, —OR⁹, —OC(O)N(R⁹)₂, —NR⁹C(O)OR⁸, —C(O)NHS(═O)₂R⁸, —S(═O)₂NHC(O)R⁸, —N(R⁹)₂, —N(R⁹)C(O)R⁸, —NR⁹C(O)N(R⁹)₂, —C(═NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂R⁹, —CON(R⁹)₂, —OS(═O)₂OR⁹, or —OP(═O)₂OR⁹. In other embodiments, G is H, tetrazolyl, —NR⁹S(═O)₂R⁸, —S(═O)₂N(R⁹)₂, —OR⁹, —C(O)NHS(═O)₂R⁸, —S(═O)₂NHC(O)R⁸, —N(R⁹)₂, —N(R⁹)C(O)R⁸, —C(═NR¹⁰)N(R⁹)₂, —NR⁹C(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂R⁹, or —CON(R)₂. In some other embodiments, G is H, tetrazolyl, —NR⁹S(═O)₂R⁸, —S(═O)₂N(R⁹)₂, —OR⁹, —C(O)NHS(═O)₂R⁸, —S(═O)₂NHC(O)R⁸, —N(R⁹)₂, —N(R⁹)C(O)R⁸, —C(═NR¹⁰)N(R⁹)₂, —NR⁹C(═NR¹⁰)N(R⁹)₂, —CO₂R⁹, or —CON(R⁹)₂. In yet some other embodiments, G is H, tetrazolyl, —NR⁹S(═O)₂R⁸, —S(═O)₂N(R⁹)₂, —OR⁹, —C(O)NHS(═O)₂R⁸, —S(═O)₂NHC(O)R⁸, —N(R⁹)₂, —N(R⁹)C(O)R⁸, —C(═NR¹⁰)N(R⁹)₂, —NR⁹C(═NR¹⁰)N(R⁹)₂, —CO₂R⁹, or —CON(R⁹)₂; each R⁸ is independently a substituted or unsubstituted C₁-C₆ alkyl; each R⁹ is H; and each R¹⁰ is independently selected from among H, —S(═O)₂R⁸, —S(═O)₂NH₂, and —C(O)R⁸.

In some embodiments, G is -L⁵-(substituted or unsubstituted heteroaryl containing 1-3 N atoms); and L⁵ is —OC(O)O—, —NHC(O)NH—, —NHC(O)O—, —O(O)CNH—, —NHC(O)—, —C(O)NH—, —C(O)O—, or —OC(O)—. In other embodiments, G is -L⁵-(substituted or unsubstituted heteroaryl containing 1-3 N atoms); and L⁵ is a bond, —OC(O)O—, —NHC(O)NH—, —NHC(O)O—, —O(O)CNH—, —NHC(O)—, —C(O)NH—, —C(O)O—, or —OC(O)—. In some other embodiments, G is -L⁵-(substituted or unsubstituted heteroaryl containing 1-3 N atoms); and L⁵ is a bond. In yet other embodiments, L is a bond, G is -L⁵-(substituted or unsubstituted heteroaryl containing 1-3 N atoms); and L⁵ is a bond. In some embodiments, G is not H.

In some embodiments, compounds provided herein have a structure of Formula (I):

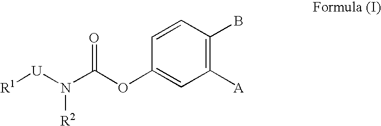

wherein:
R¹ is selected from the group consisting of:

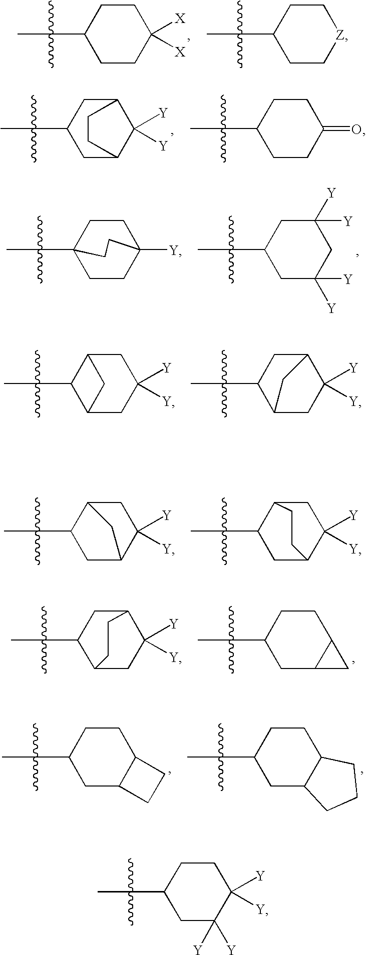

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
one of A Or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁹, —S(═O)₂NH-phenyl, —OH, —SH, —OC(O)NHR⁹, —NHC(O)OR⁸, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R)₂, —NR⁹C(═NR¹⁰)NHC(═NR¹⁰)N(R)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —OS(═O)₂OH, —OP(═O)₂OH, or -L⁵-(substituted or unsubstituted heteroaryl containing 1-3 N atoms);
L⁵ is a bond, —OC(O)O—, —NHC(O)NH—, —NHC(O)O—, —O(O)CNH—, —NHC(O)—, —C(O)NH—, —C(O)O—, or —OC(O)—;
each R⁸ is independently a substituted or unsubstituted C₁-C₆ alkyl;
each R⁹ is independently H, a substituted C₁-C₆ alkyl or unsubstituted C₁-C₆ alkyl; and
each R¹⁰ is independently selected from among H, —S(═O)₂R⁸, —S(═O)₂NH₂, —C(O)R⁸, —CN, and —NO₂;
n is 1, 2, 3, or 4; and
pharmaceutically acceptable salts, pharmaceutically acceptable N-oxides, pharmaceutically active metabolites, pharmaceutically acceptable prodrugs, or pharmaceutically acceptable solvates thereof.

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G.

In certain embodiments, R² is H.

In some embodiments, U is a bond. In other embodiments, U is CH₂.

In some embodiments, L is a bond, or an optionally substituted group selected from among C₁-C₆ alkylene, C₁-C₆ ketoalkylene, a monosaccharide, a disaccharide, —C(O)NR⁹—(CH₂)ₙ—, —NR⁹—C(O)—(CH₂)ₙ—, —NR⁹C(O)N(R⁹)—(CH₂)ₙ, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—. In other embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, —NR⁹C(O)N(R⁹)—(CH₂)ₙ—, —S(O)—(CH₂)ₙ—, and —S(O)₂—(CH₂)ₙ—. In some other embodiments, L is a bond, or an optionally substituted group selected from among C₁-C₆ alkylene, C₁-C₆ ketoalkylene, —C(O)NR₉—(CH₂)ₙ—, —NR₉—C(O)—(CH₂)ₙ—, and —NR⁹C(O)N(R⁹)—(CH₂)ₙ—. In other embodiments, L is a bond, or an optionally substituted group selected from among C₁-C₆ alkyl and C₁-C₆ ketoalkyl. In some embodiments, L is a bond.

In some embodiments, G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂).), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁹, —S(═O)₂NH-phenyl, —OH, —SH, —OC(O)NHR⁹, —NHC(O)OR⁸, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —OS(═O)₂OH, or —OP(═O)₂OH. In other embodiments, G is H, tetrazolyl, —NHS(═O)₂R⁸, —S(═O)₂NHR⁹, —S(═O)₂NH-phenyl, —OH, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹¹)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, or —CO₂H. In some other embodiments, G is H, tetrazolyl, —NHS(═O)₂R⁸, —S(═O)₂NHR⁹, —S(═O)₂NH-phenyl, —OH, —C(O)NHC(O)R⁸, —C(O)NHS(═O)₂R⁸, —S(═O)₂NHC(O)R⁸, —S(═O)₂NHC(O)NHR⁹, —N(R⁹)₂, —NHC(O)R⁸, —NHC(O)N(R⁹)₂, —C(═NR¹⁰)N(R⁹)₂, —NR⁹C(═NR¹⁰)N(R⁹)₂, or —CO₂H. In yet other embodiments, G is H, tetrazolyl, —NHS(═O)₂R⁸, —S(═O)₂NHR⁹, —S(═O)₂NH-phenyl, —OH, —C(O)NHS(═O)₂R⁸, —S(═O)₂NHC(O)R⁸, —N(R)₂, —NHC(O)R⁸, —NHC(O)N(R⁹)₂, —C(═NR¹⁰)N(R⁹)₂, —NR⁹C(═NR¹⁰)N(R⁹)₂, or —CO₂H; each R⁸ is independently a substituted or unsubstituted C₁-C₆ alkyl; each R⁹ is H; and each R¹⁰ is independently selected from among H, —S(═O)₂R⁸, —S(═O)₂NH₂, and —C(O)R⁸.

In some embodiments, each R⁸ is independently a substituted or unsubstituted C₁-C₆ alkyl; each R⁹ is H; and each R¹⁰ is independently selected from among H, —S(═O)₂R⁸, and —C(O)R⁸. In some embodiments, each R⁹ is H.

In some embodiments, G is -L⁵-(substituted or unsubstituted heteroaryl containing 1-3 N atoms); and L⁵ is a bond, —OC(O)O—, —NHC(O)NH—, —NHC(O)O—, —O(O)CNH—, —NHC(O)—, —C(O)NH—, —C(O)O—, or —OC(O)—. In some embodiments, G is -L⁵-(substituted or unsubstituted heteroaryl containing 1-3 N atoms); and L⁵ is a bond. In some embodiments, G is not H.

Further compounds provided herein have a structure selected from among:

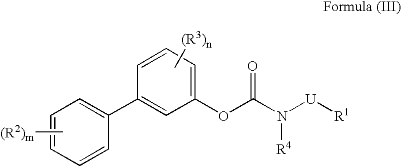

wherein:
R¹ is selected from the group consisting of:

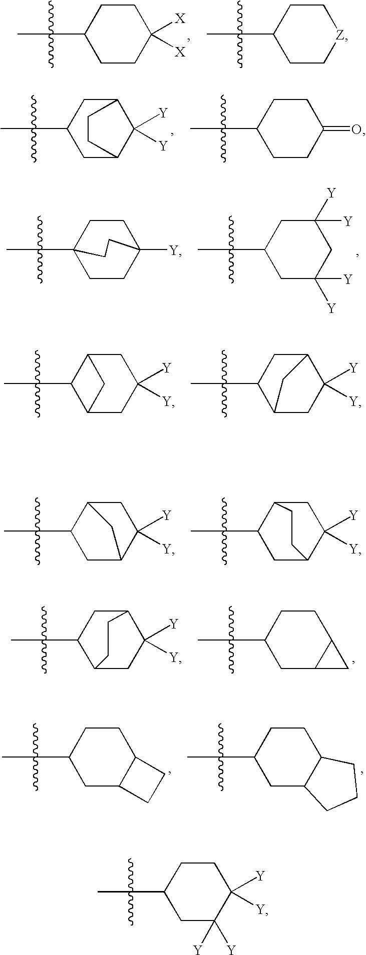

neopentyl, neohexyl, methylenecyclopropyl, methylenecyclobutyl, and methylenecyclopentyl; with the proviso that R¹ is not unsubstituted cyclohexyl;
R⁴ is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N—(C₁-C₆ alkyl), or SO₂;
U is a bond or CH₂;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), arloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl; and m and n are each independently 0-3; and
pharmaceutically acceptable salts, pharmaceutically acceptable N-oxides, pharmaceutically active metabolites, pharmaceutically acceptable prodrugs, or pharmaceutically acceptable solvates thereof.

In some embodiments, the compound of Formula (III) has the structure:

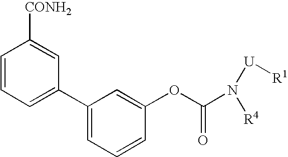

wherein:
R¹ is selected from the group consisting of:

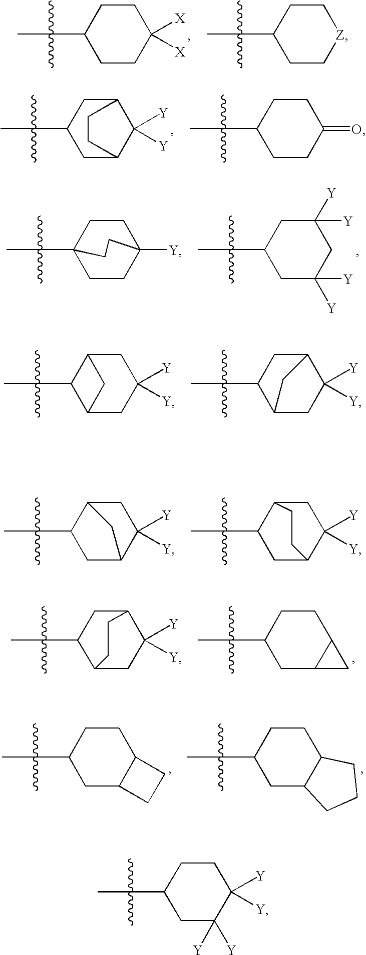

neopentyl, neohexyl, methylenecyclopropyl, methylenecyclobutyl, and methylenecyclopentyl; with the proviso that R¹ is not unsubstituted cyclohexyl;
R⁴ is H or an optionally substituted alkyl;
U is a bond or CH₂; and
   pharmaceutically acceptable salts, pharmaceutically acceptable N-oxides, pharmaceutically active metabolites, pharmaceutically acceptable prodrugs, or pharmaceutically acceptable solvates thereof.

Further compounds described herein are:

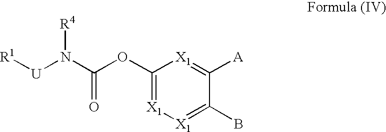

wherein:
R¹ is selected from the group consisting of:

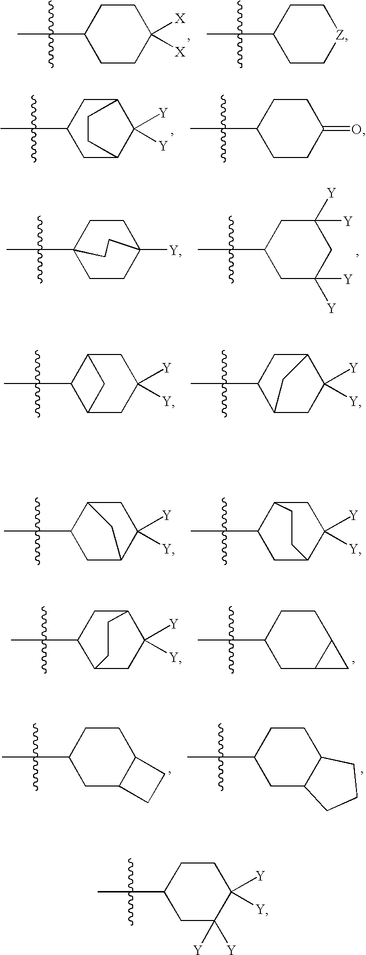

neopentyl, neohexyl, methylenecyclopropyl, methylenecyclobutyl, and methylenecyclopentyl; with the proviso that R¹ is not unsubstituted cyclohexyl;
R⁴ is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;

A and B are selected from:
(i) one of A or B is C(O)-alkyl or —C(O)N(alkyl)((CH₂)ₙG), and the other is H, alkyl, heteroalkyl;
   A and B can combine into a non-aromatic cyclic group;
   A and B can be substituted; or
(ii) A and B together form an optionally substituted heteroaromatic group;
   A and/or B are N, S, O, or CR⁹; or
(iii) A and B are H provided that at least one X₁ is not CH;

each X₁ is independently CH or N; and n is 1, 2, 3, or 4; and
pharmaceutically acceptable salts, pharmaceutically acceptable N-oxides, pharmaceutically active metabolites, pharmaceutically acceptable prodrugs, or pharmaceutically acceptable solvates thereof.

Further compounds described herein have the structure of Formula (1), Formula (2), Formula (3), Formula (4), Formula (5), Formula (6), Formula (7), Formula (8), Formula (9), Formula (10), Formula (11), Formula (12), Formula (13), Formula (14), Formula (15), Formula (16), Formula (17), Formula (18), Formula (19), Formula (20), Formula (21), Formula (22), Formula (23), Formula (24), Formula (25), Formula (26), Formula (27), Formula (28), or Formula (29) disclosed in U.S. Provisional Patent Application No. 60/755,035, filed on Dec. 29, 2005, herein incorporated by reference; provided that the R¹ group has the structure:

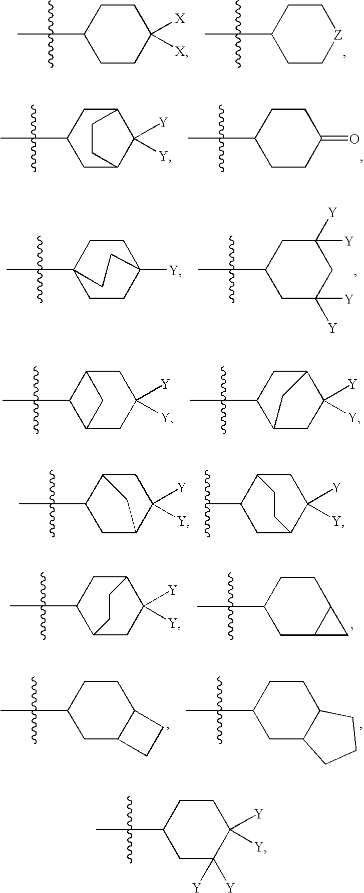

neopentyl, neohexyl, methylenecyclopropyl, methylenecyclobutyl, and methylenecyclopentyl; with the proviso that R¹ is not unsubstituted cyclohexyl;
   each X is independently halogen, methyl, fluoromethyl, or each X taken together can form a 3-, 4-, or 5-membered carbocyclic group;
   each Y is independently H, halogen, methyl, fluoromethyl, or each Y taken together can form a 3-, 4-, or 5-membered carbocyclic group; and
   Z is O, N—(C₁-C₆ alkyl), or SO₂; and
   pharmaceutically acceptable salts, pharmaceutically acceptable N-oxides, pharmaceutically active metabolites, pharmaceutically acceptable prodrugs, or pharmaceutically acceptable solvates thereof.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments, compounds provided herein have a structure selected from among:

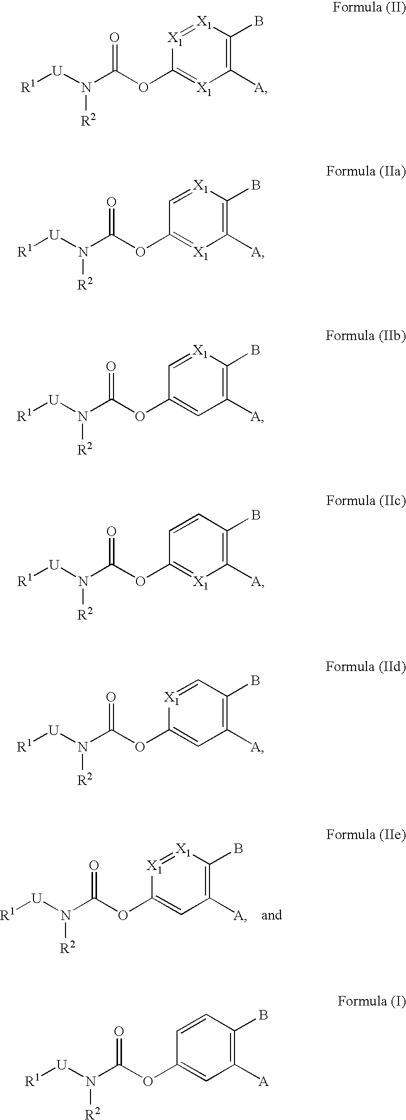

wherein:
R¹ is selected from the group consisting of:

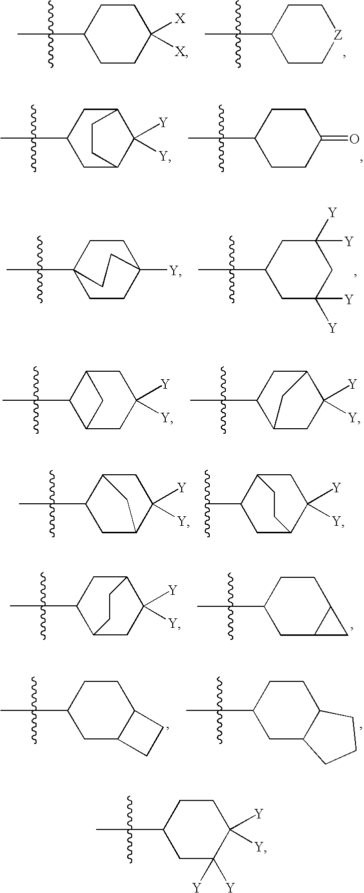

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is -L-G and the other is selected from among H and an optionally substituted C₁-C₆ alkyl; or
A and B are both selected from among H and an optionally substituted C₁-C₆ alkyl provided that at least one X₁ is present and is N;
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)ₙ—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹)N(R⁹)—(CH₂)ₙ—;
G is tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(—NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₓOH, —OP(═O)OR⁸OH, —OP(═O)R⁸OH, —NR⁹P(═O)OR⁸OH, NR⁹P(═O)R¹⁰H, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
X₁ is independently CH or N, provided that at least one X₁ is N;
n is 1, 2, 3, or 4; x is 1, 2, or 3; y is 0, 1, or 2; and pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure of Formula (II):

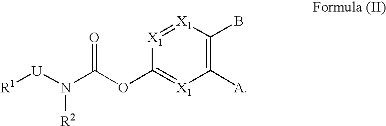

In some embodiments, compounds provided herein have a structure of Formula (IIa):

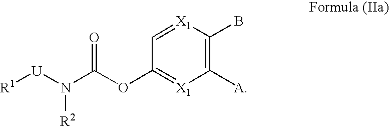

In other embodiments, compounds provided herein have a structure of Formula (IIb):

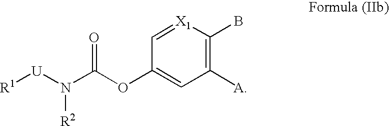

In some other embodiments, compounds provided herein have a structure of Formula (IIc):

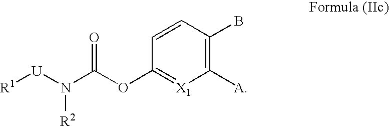

In another embodiment, compounds provided herein have a structure of Formula (IId):

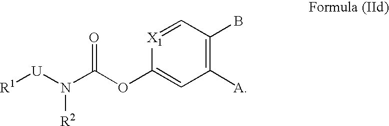

In yet some other embodiments, compounds provided herein have a structure of Formula (IIe):

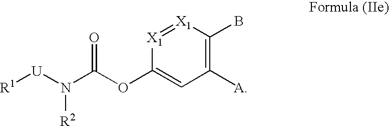

In some embodiments, both A and B are H provided that at least one X₁ is present and is N. In embodiments where X₁ is present, at least one X₁ is N.

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G. In some other embodiments, both A and B are selected from among H and an optionally substituted C₁-C₆ alkyl provided that at least one X₁ is present and is N.

In certain embodiments, R² is H.

In some embodiments, U is a bond. In other embodiments, U is CH₂.

In some embodiments, L is a bond, or an optionally substituted group selected from among C₁-C₆alkylene, C₁-C₆ ketoalkylene, a monosaccharide, a disaccharide, —C(O)NR⁹—(CH₂)ₙ—, —NR⁹—C(O)—(CH₂)ₙ—, or —S(O)₂—(CH₂)ₙ—.

In other embodiments, L is a bond. In some embodiments, if L is not a bond, L taken together with A or 13 can form a carbocyclic ring.

In certain embodiments, G is tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH 2-CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R)₂, —C(O)NR⁹C(═NR¹⁰)N(R⁹)₂, or —C(O)NR⁹C(═CHR¹⁰)N(R⁹)₂. In some embodiments, each R¹⁰ is H.

In other embodiments, G is —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —CO₂H, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; or —NR⁹S(O)_{y}OH. In some other embodiments, G is —S(═O)₂NH-phenyl, —C(O)NHC(O)R⁸, —C(O)NHS(═O)₂R⁸, —S(═O)₂NHC(O)R⁸, —S(═O)₂NHC(O)NHR⁸, —CO₂H, —(OP(═O)OH)ₓOH, —OP(═O)OR⁸OH, —OP(═O)R⁸OH, —P(═O)OR⁸OH; —P(═O)R⁸OH, or —S(O)_{y}OH. In yet other embodiments, G is —S(═O)₂NH-phenyl, —C(O)NHC(O)R⁸, —C(O)NHS(═O)₂R⁸, —S(═O)₂NHC(O)R⁸, —S(═O)₂NHC(O)NHR⁸, —CO₂H, —(OP(═O)OH)ₓOH, or —S(O)_{y}OH; x is 1; and y is 1 or 2.

In some embodiments, each R⁸ is independently a substituted or unsubstituted C₁-C₆ alkyl; each R⁹ is H; and each R¹⁰ is independently selected from among H, —S(═O)₂R⁸, and —C(O)R⁸. In some embodiments, each R⁹ is H. In some other embodiments, each R¹⁰ is H.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments, compounds provided herein have a structure of Formula (I):

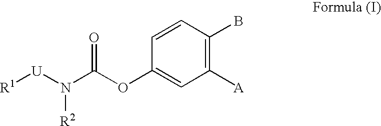

wherein:
R¹ is selected from the group consisting of:

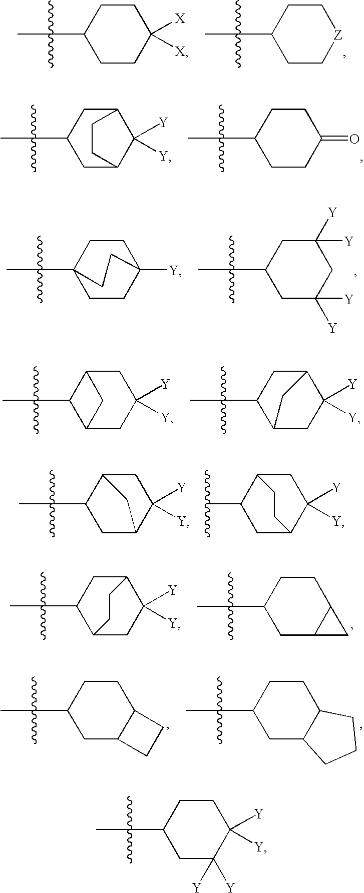

neopentyl, neohexyl, methylenecyclopropyl, methylenecyclobutyl, and methylenecyclopentyl; with the proviso that R¹ is not unsubstituted cyclohexyl;
R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is -L-G and the other is selected from among H and an optionally substituted C₁-C₆ alkyl;
L is a bond, or an optionally substituted group selected from among C₁-C₆ alkylene, C₁-C₆ heteroalkylene, C₁-C₆ ketoalkylene, a monosaccharide, a disaccharide, —C(O)NR⁹—(CH₂)ₙ—, —NR⁹—C(O)—(CH₂)—, —OC(O)O—(CH₂)ₙ—, —NHC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O—(CH₂—CH₂—O)_{q}—H, —CH₂—(O—CHR_{M}—CHR_{M})_{q}—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R⁹)₂, —(C(H)_{y}—((C₁-C₆)N(R⁹)₂).), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹)N(R)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently a substituted or unsubstituted C₁-C₆ alkyl;
each R⁹ is independently H, a substituted C₁-C₆ alkyl or unsubstituted C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 0, 1, or 2; and

pharmaceutically acceptable salts, pharmaceutically acceptable N-oxides, pharmaceutically active metabolites, pharmaceutically acceptable prodrugs, or pharmaceutically acceptable solvates thereof.

For any and all of the embodiments, substituents can be selected from among from a subset of the listed alternatives. For example, in some embodiments, one of A or B is -L-G and the other is H or an optionally substituted C₁-C₆ alkyl. In other embodiments, one of A or B is -L-G and the other is H. In some embodiments, A is -L-G. In some other embodiments, B is -L-G.

In certain embodiments, R² is H.

In some embodiments, L is a bond, or an optionally substituted group selected from among C₁-C₆ alkylene, C₁-C₆ ketoalkylene, a monosaccharide, a disaccharide, —C(O)NR⁹—(CH₂)ₙ—, —NR⁹—C(O)—(CH₂)ₙ—, or —S(O)₂—(CH₂)ₙ—.

In other embodiments, L is a bond. In some embodiments, if L is not a bond, L taken together with A or B can form a carbocyclic ring.

In certain embodiments, G is H, tetrazolyl, —CH₂—(O—CH₂—CH₂)_{q}—O—CH₃, —O—(CH₂—CH₂—O)_{q}—CH₃, —CH₂—(O—CH₂—CH₂)_{q}—OH, —O(CH₂—CH₂—O)_{q}—H, —CH₂—(O—CHR_{M}—CHR_{M})_{q}—O—CH₃ or —O—(CHR_{M}—CHR_{M}—O)_{q}—CH₃, —CH₂—(O—CHR_{M}—CHR_{M})_{q}—OH or —O—(CHR_{M}—CHR_{M}—O)_{q}—H, wherein one of R_{M} is methyl and the other R_{M} is H, and q is an integer between 1 and 300; —(C₁-C₆)—N(R)₂, —(C(H)_{y}—((C₁-C₆)N(R⁹)₂)ₓ), an amino acid having the 3-letter code selected from Ala, Arg, Asn, Asp, Cys, Glu, Gln, Gly, His, Ile, Leu, Lys, Met, Phe, Pro, Ser, Thr, Trp, Tyr, and Val attached at either the amine portion or the carboxylate portion, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, or —C(O)NR⁹C(═CHR¹⁰)N(R⁹)₂. In some embodiments, each R¹⁰ is H.

In other embodiments, G is —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —CO₂H, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; or —NR⁹S(O)_{y}OH. In some other embodiments, G is —S(═O)₂NH-phenyl, —C(O)NHC(O)R⁸, —C(O)NHS(═O)₂R⁸, —S(═O)₂NHC(O)R⁸, —S(═O)₂NHC(O)NHR⁸, —CO₂H, —(OP(═O)OH)ₓOH, —OP(═O)OR⁸OH, —OP(═O)R⁸OH, —P(═O)OR⁸OH; —P(═O)R⁸OH, or —S(O)_{y}OH. In yet other embodiments, G is —S(═O)₂NH-phenyl, —C(O)NHC(O)R⁸, —C(O)NHS(═O)₂R⁸, —S(═O)₂NHC(═O)R⁸, —S(═O)₂NHC(O)NHR⁸, —CO₂H, —(OP(═O)OH)ₓOH, or —S(O)_{y}OH; x is 1; and y is 1 or 2.

In some embodiments, are compounds of Formula (I) wherein:

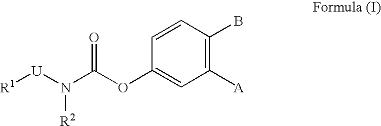

R¹ is selected from the group consisting of:

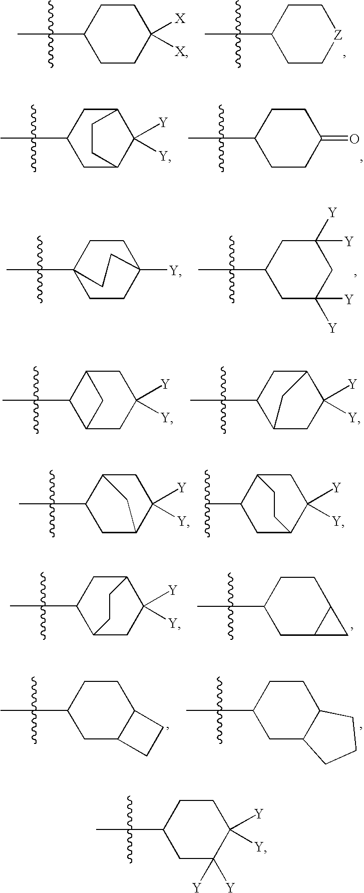

neopentyl, neohexyl, methylenecyclopropyl, methylenecyclobutyl, and methylenecyclopentyl, with the proviso that R¹ is not substituted cyclohexyl;
R² is H or an optionally substituted alkyl;
   each X is independently halogen, methyl, fluoromethyl, or each X taken together can form a 3-, 4-, or 5-membered carbocyclic group;
   each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is (CH₂)_{q}C(O)—N(R²)₂ and the other is H; q is 0 or 1;
wherein each optional substituent is independently selected from C₁-C₃ alkyl, C₁-C₃ alkoxy, benzyl, halogen, nitro, cyano, or benzyloxy —C(O)R′, —C(O)-(alkyl or substituted alkyl), -(alkyl or substituted alkyl)-C(O)R′, —C(O)N(R′)₂, —C(O)N(R′)-(alkyl or substituted alkyl), -(alkyl or substituted alkyl)-C(O)N(R′)₂, —OC(O)N(R′)₂, —OC(O)N(R′)-(alkyl or substituted alkyl), -(alkyl or substituted alkyl)-OC(O)N(R′)₂, —N(R′)C(O)R′, —NR′C(O)-(alkyl or substituted alkyl), -(alkyl or substituted alkyl)-NR′C(O)R′, —SR′, —S-(alkyl or substituted alkyl), —S(O)ₖR′, where k is 1, or 2, —S(O)ₖ(alkyl or substituted alkyl), —C(S)-(alkyl or substituted alkyl), —CSN(R′)₂, —CSN(R′)-(alkyl or substituted alkyl), —N(R′)CO-(alkyl or substituted alkyl), —N(R′)C(O)OR′, -(alkyl or substituted alkyl)-O—N═C(R′)₂, -(alkyl or substituted alkyl)-C(O)NR′-(alkyl or substituted alkyl), -(alkyl or substituted alkyl)-S(O)ₖ-(alkyl or substituted alkyl)-SR′, -(alkyl or substituted alkyl)-S—SR′, —S(O)ₖN(R′)₂, —N(R′)C(O)N(R′)₂, —N(R′)C(S)N(R′)₂, —N(R′)S(O)ₖN(R′)₂, —C(R′)═NR′—C(R′)═N—N(R′)₂, and —C(R′)₂—N(R′)—N(R′)₂; and

pharmaceutically acceptable salts, pharmaceutically acceptable N-oxides, pharmaceutically active metabolites, pharmaceutically acceptable prodrugs, or pharmaceutically acceptable solvates thereof.

In one embodiment is a compound of Formula (I) wherein:

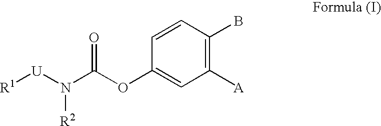

wherein:

one of A or B is -L-G and the other is H;

L is —C(O)NR⁹—(CH₂)ⱼ—;

R⁹ is independently H, a substituted C₁-C₆ alkyl or unsubstituted C₁-C₆ alkyl;

j is 0;

G is H;

R¹ is selected from the group consisting of:

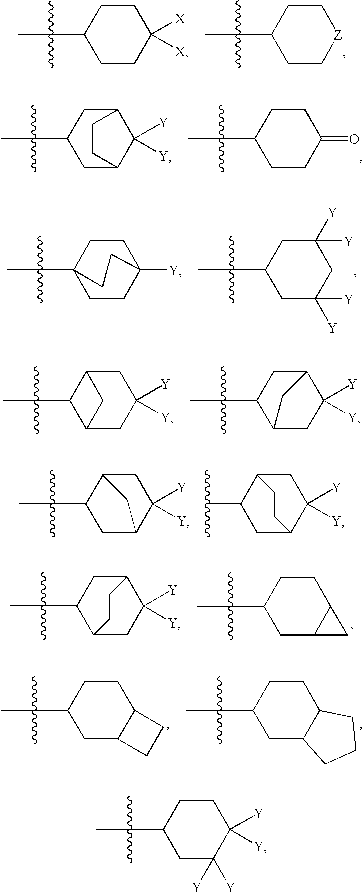

neopentyl, neohexyl, methylenecyclopropyl, methylenecyclobutyl, and methylenecyclopentyl, with the proviso that R¹ is not unsubstituted cyclohexyl;

R² is H;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂; and

pharmaceutically acceptable salts, pharmaceutically acceptable N-oxides, pharmaceutically active metabolites, pharmaceutically acceptable prodrugs, or pharmaceutically acceptable solvates thereof.

In a further embodiment, is a compound of Formula (I) selected from the group consisting of:

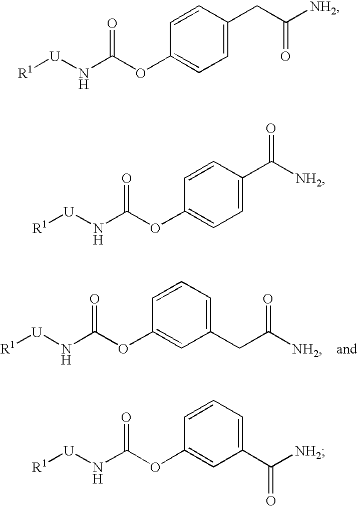

wherein:

R¹ is selected from the group consisting of:

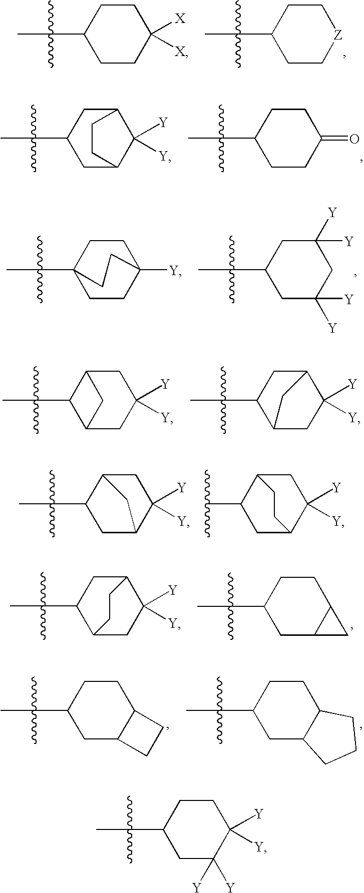

neopentyl, neohexyl, methylenecyclopropyl, methylenecyclobutyl, and methylenecyclopentyl, with the proviso that R¹ is not unsubstituted cyclohexyl;

U is a bond or CH₂; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment is a compound of Formula (I):

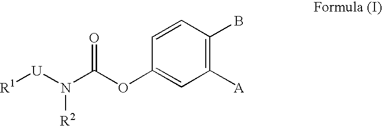

wherein:

R¹ is selected from the group consisting of:

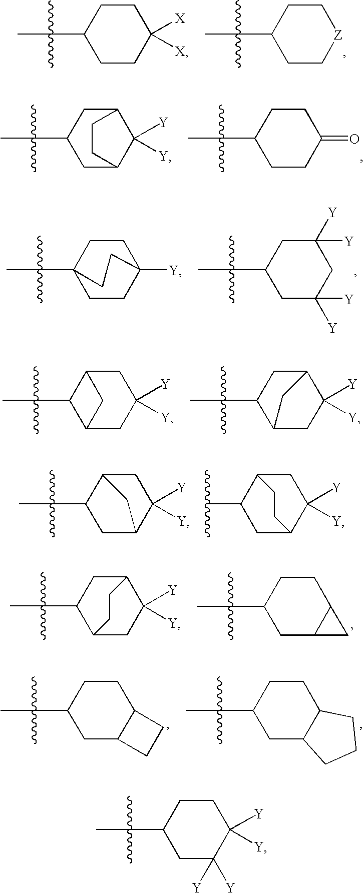

neopentyl, neohexyl, methylenecyclopropyl, methylenecyclobutyl, and methylenecyclopentyl, with the proviso that R¹ is not unsubstituted cyclohexyl;

R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂;

one of A or B is -L-G and the other is selected from among H and an optionally substituted C₁-C₆ alkyl;

L is a bond;

G is —CO₂H;

wherein each optional substituent is independently selected from C₁-C₃ alkyl, C₁-C₃ alkoxy, benzyl, halogen, nitro, cyano, or benzyloxy —C(O)R′, —C(O)-(alkyl or substituted alkyl), -(alkyl or substituted alkyl)-C(O)R′, —C(O)N(R′)₂, —C(O)N(R′)-(alkyl or substituted alkyl), -(alkyl or substituted alkyl)-C(O)N(R′)₂, —OC(O)N(R′)₂, —OC(O)N(R′)-(alkyl or substituted alkyl), -(alkyl or substituted alkyl)-OC(O)N(R′)₂, —N(R′)C(O)R′, —NR′C(O)-(alkyl or substituted alkyl), -(alkyl or substituted alkyl)-NR′C(O)R′, —SR′, —S-(alkyl or substituted alkyl), —S(O)ₖR′, where k is 1, or 2, —S(O)ₖ(alkyl or substituted alkyl), —C(S)-(alkyl or substituted alkyl), —CSN(R′)₂, —CSN(R′)-(alkyl or substituted alkyl), —N(R′)CO-(alkyl or substituted alkyl), —N(R′)C(O)OR′, -(alkyl or substituted alkyl)-O—N═C(R′)₂, -(alkyl or substituted alkyl)-C(O)NR′-(alkyl or substituted alkyl), -(alkyl or substituted alkyl)-S(O)ₖ-(alkyl or substituted alkyl)-SR′, -(alkyl or substituted alkyl)-S—SR′, —S(O)ₖN(R′)₂, —N(R′)C(O)N(R′)₂, —N(R′)C(S)N(R′)₂, —N(R′)S(O)ₖN(R′)₂, —C(R′)═NR′-C(R′)═N—N(R′)₂, and —C(R′)₂—N(R′)—N(R′)₂; and

pharmaceutically acceptable salts, pharmaceutically acceptable N-oxides, pharmaceutically active metabolites, pharmaceutically acceptable prodrugs, or pharmaceutically acceptable solvates thereof. In one embodiment, R¹ is selected from the group consisting of:

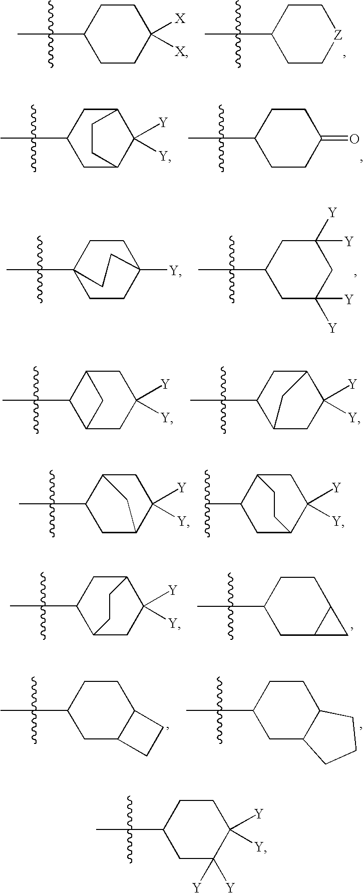

neopentyl, neohexyl, methylenecyclopropyl, methylenecyclobutyl, and methylenecyclopentyl, with the proviso that R¹ is not unsubstituted cyclohexyl; each X is independently halogen, methyl, fluoromethyl, or each X taken together can form a 3-, 4-, or 5-membered carbocyclic group; each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂; R² is H;

L is a bond; G is —CO₂H; and pharmaceutically acceptable salts, pharmaceutically acceptable N-oxides, pharmaceutically active metabolites, pharmaceutically acceptable prodrugs, or pharmaceutically acceptable solvates thereof.

In another embodiment, one of A or B is -L-G and the other is selected from among H and an optionally substituted C₁-C₆ alkyl;

L is an optionally substituted group selected from among C₁-C₆ alkylene;

G is —CO₂H;

wherein each optional substituent is independently selected from C₁-C₃ alkyl, C₁-C₃ alkoxy, benzyl, halogen, nitro, cyano, or benzyloxy —C(O)R′, —C(O)-(alkyl or substituted alkyl), -(alkyl or substituted alkyl)-C(O)R′, —C(O)N(R′)₂, —C(O)N(R′)-(alkyl or substituted alkyl), -(alkyl or substituted alkyl)-C(O)N(R′)₂, —OC(O)N(R′)₂, —OC(O)N(R′)-(alkyl or substituted alkyl), -(alkyl or substituted alkyl)-OC(O)N(R′)₂, —N(R′)C(O)R′, —NR′C(O)-(alkyl or substituted alkyl), -(alkyl or substituted alkyl)-NR′C(O)R′, —SR′, —S-(alkyl or substituted alkyl), —S(O)ₖR′, where k is 1, or 2, —S(O)ₖ(alkyl or substituted alkyl), —C(S)-(alkyl or substituted alkyl), —CSN(R′)₂, —CSN(R′)-(alkyl or substituted alkyl), —N(R′)CO-(alkyl or substituted alkyl), —N(R′)C(O)OR′, -(alkyl or substituted alkyl)-O—N═C(R′)₂, -(alkyl or substituted alkyl)-C(O)NR′-(alkyl or substituted alkyl), -(alkyl or substituted alkyl)-S(O)ₖ-(alkyl or substituted alkyl)-SR′, -(alkyl or substituted alkyl)-S—SR′, —S(O)ₖN(R′)₂, —N(R′)C(O)N(R′)₂, —N(R′)C(S)N(R′)₂, —N(R′)S(O)ₖN(R′)₂, —C(R′)═NR′-C(R′)═N—N(R′)₂, and —C(R′)₂—N(R′)—N(R′)₂; and

pharmaceutically acceptable salts, pharmaceutically acceptable N-oxides, pharmaceutically active metabolites, pharmaceutically acceptable prodrugs, or pharmaceutically acceptable solvates thereof. In another embodiment, L is CH₂; G is —CO₂H; and pharmaceutically acceptable salts, pharmaceutically acceptable N-oxides, pharmaceutically active metabolites, pharmaceutically acceptable prodrugs, or pharmaceutically acceptable solvates thereof.

In other embodiments, compounds provided herein have a structure selected from among:

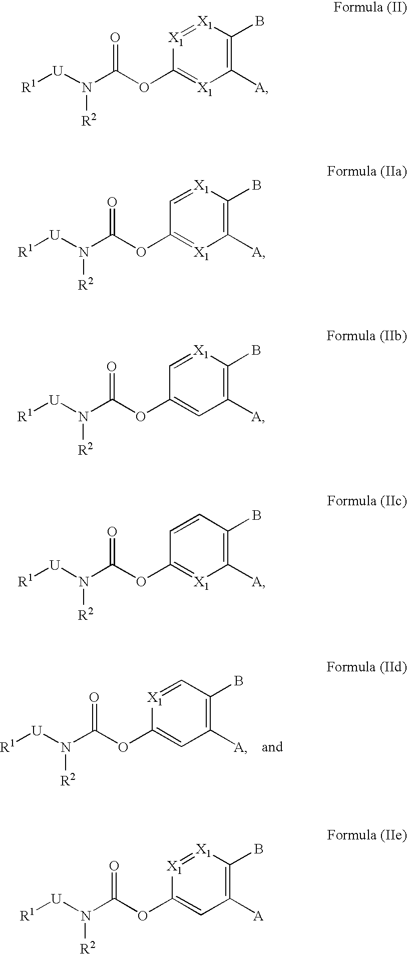

wherein:
R¹ is selected from the group consisting of:

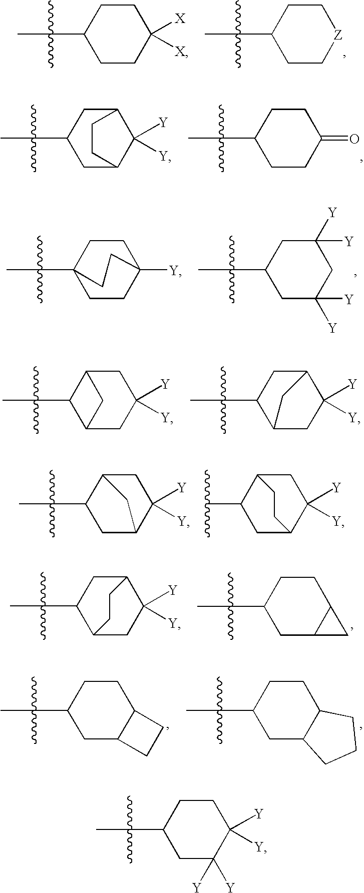

neopentyl, neohexyl, methylenecyclopropyl, methylenecyclobutyl, and methylenecyclopentyl; with the proviso that R¹ is not unsubstituted cyclohexyl;
R² is H;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is -L-G and the other is H;
L is a bond;
G is —CO₂R⁹;
R⁹ is H;
each X₁ is CH; and

pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure selected from among:

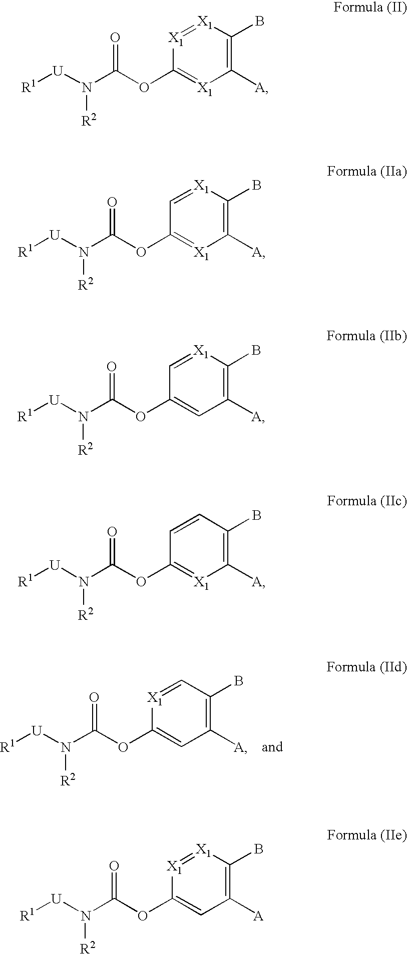

wherein:
R¹ is selected from the group consisting of:

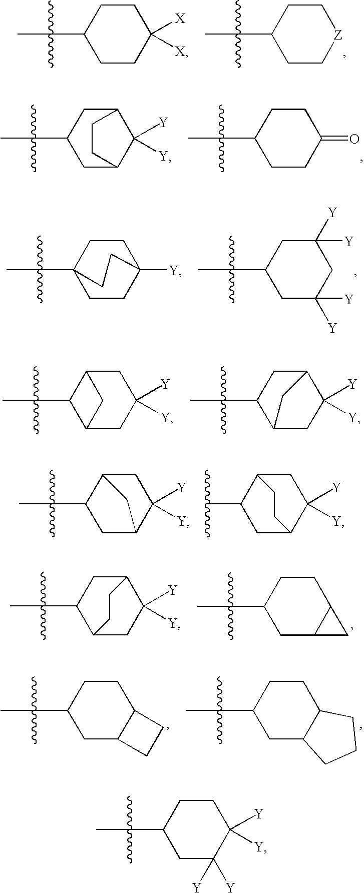

neopentyl, neohexyl, methylenecyclopropyl, methylenecyclobutyl, and methylenecyclopentyl; with the proviso that R¹ is not unsubstituted cyclohexyl;
R² is H;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is -L-G and the other is H;
L is CH₂;
G is —CO₂R⁹;
R⁹ is H;
each X₁ is CH; and

pharmaceutically acceptable salts, pharmaceutically acceptable N-oxides, pharmaceutically active metabolites, pharmaceutically acceptable prodrugs, or pharmaceutically acceptable solvates thereof.

In one embodiment, is a compound of Formula (I) selected from the group consisting of:

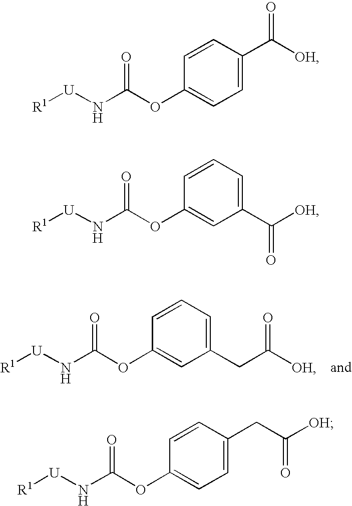

wherein:

R¹ is selected from the group consisting of:

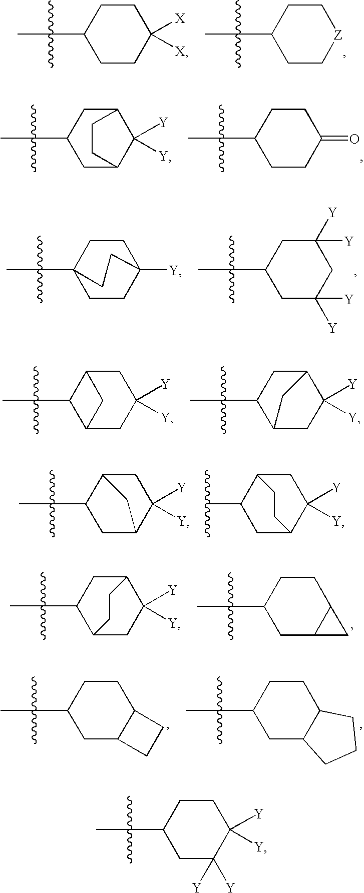

neopentyl, neohexyl, methylenecyclopropyl, methylenecyclobutyl, and methylenecyclopentyl, with the proviso that R¹ is not unsubstituted cyclohexyl;

U is a bond or CH₂; and

pharmaceutically acceptable salts, pharmaceutically acceptable N-oxides, pharmaceutically active metabolites, pharmaceutically acceptable prodrugs, or pharmaceutically acceptable solvates thereof.

In one embodiment is a compound of Formula (I):

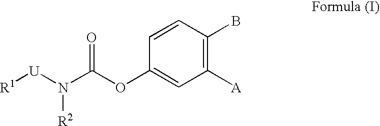

wherein:

R¹ is selected from the group consisting of:

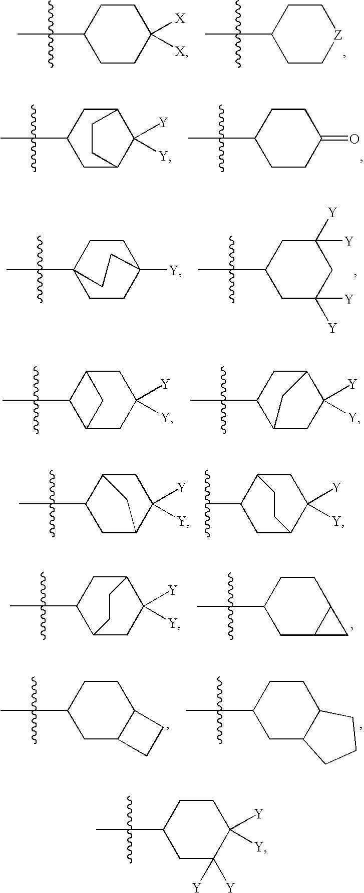

neopentyl, neohexyl, methylenecyclopropyl, methylenecyclobutyl, and methylenecyclopentyl, with the proviso that R¹ is not unsubstituted cyclohexyl;

R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂;

A and B are each independently selected from among H and an optionally substituted amide;

wherein each optional substituent is independently selected from C₁-C₃ alkyl, C₁-C₃ alkoxy, benzyl, halogen, nitro, cyano, or benzyloxy —C(O)R′, —C(O)-(alkyl or substituted alkyl), -(alkyl or substituted alkyl)-C(O)R′, —C(O)N(R′)₂, —C(O)N(R′)-(alkyl or substituted alkyl), -(alkyl or substituted alkyl)-C(O)N(R′)₂, —OC(O)N(R′)₂, —OC(O)N(R′)-(alkyl or substituted alkyl), -(alkyl or substituted alkyl)-OC(O)N(R′)₂, —N(R′)C(O)R′, —NR′C(O)-(alkyl or substituted alkyl), -(alkyl or substituted alkyl)-NR′C(O)R′, —SR′, —S-(alkyl or substituted alkyl), —S(O)ₖR′, where k is 1, or 2, —S(O)ₖ(alkyl or substituted alkyl), —C(S)-(alkyl or substituted alkyl), —CSN(R′)₂, —CSN(R′)-(alkyl or substituted alkyl), —N(R′)CO-(alkyl or substituted alkyl), —N(R′)C(O)OR′, -(alkyl or substituted alkyl)-O—N═C(R′)₂, -(alkyl or substituted alkyl)-C(O)NR′-(alkyl or substituted alkyl), -(alkyl or substituted alkyl)-S(O)ₖ-(alkyl or substituted alkyl)-SR′, -(alkyl or substituted alkyl)-S—SR′, —S(O)ₖN(R′)₂, —N(R′)C(O)N(R′)₂, —N(R′)C(S)N(R′)₂, —N(R′)S(O)ₖN(R′)₂, —C(R′)═NR′-C(R′)═N—N(R′)₂, and —C(R′)₂—N(R′)—N(R′)₂; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment, is a compound of Formula (I) wherein one of A or B is -L-G and the other is selected from among H and an optionally substituted C₁-C₆ alkyl; wherein L is a bond and G is —NHC(O)R⁸; R⁸ is an unsubstituted C₁-C₆ alkyl; and pharmaceutically acceptable salts, pharmaceutically acceptable N-oxides, pharmaceutically active metabolites, pharmaceutically acceptable prodrugs, or pharmaceutically acceptable solvates thereof. In another embodiment, R⁸ is CH₃.

In some embodiments, compounds provided herein have a structure selected from among:

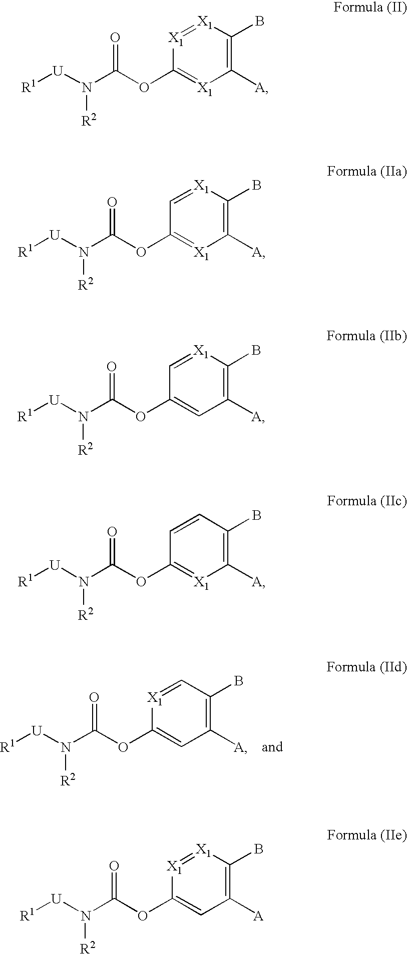

wherein:
R¹ is selected from the group consisting of:

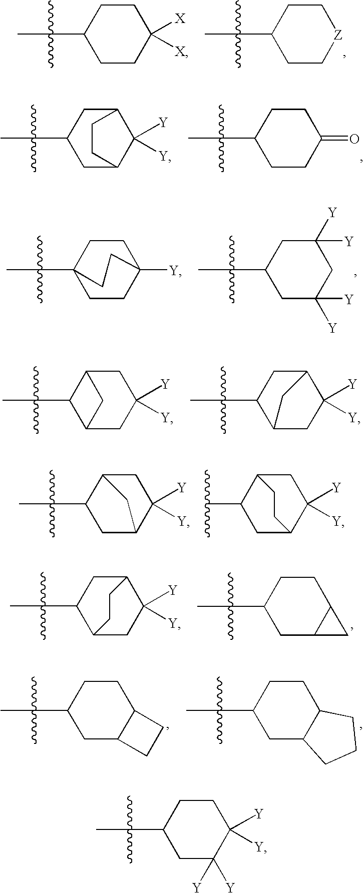

neopentyl, neohexyl, methylenecyclopropyl, methylenecyclobutyl, and methylenecyclopentyl; with the proviso that R¹ is not unsubstituted cyclohexyl;
R² is H;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is -L-G and the other is H;
L is —NR⁹—C(O)—(CH₂)ₙ—;
G is H;
R⁹ is H;
each X₁ is CH;
n is 1; and

pharmaceutically acceptable salts, pharmaceutically acceptable N-oxides, pharmaceutically active metabolites, pharmaceutically acceptable prodrugs, or pharmaceutically acceptable solvates thereof.

In a further embodiment, is a compound selected from the group consisting of:

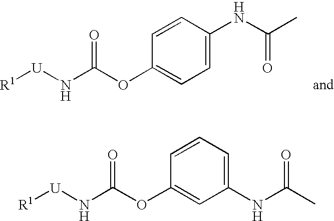

wherein:

R¹ is selected from the group consisting of:

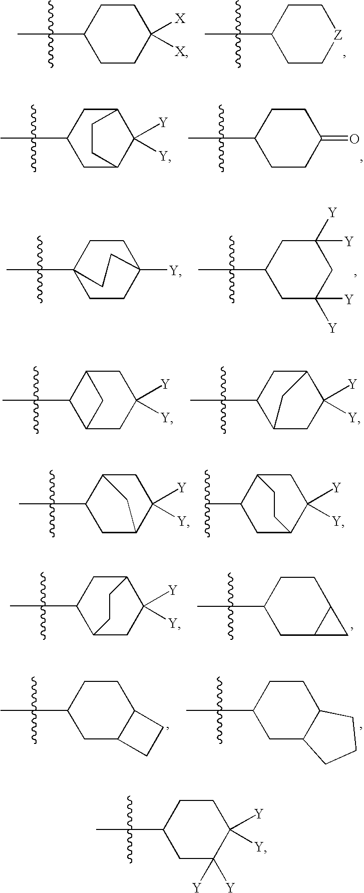

neopentyl, neohexyl, methylenecyclopropyl, methylenecyclobutyl, and methylenecyclopentyl, with the proviso that R¹ is not unsubstituted cyclohexyl;

U is a bond or CH₂; and

pharmaceutically acceptable salts, pharmaceutically acceptable N-oxides, pharmaceutically active metabolites, pharmaceutically acceptable prodrugs, or pharmaceutically acceptable solvates thereof.

In one embodiment, is a compound of Formula (I)

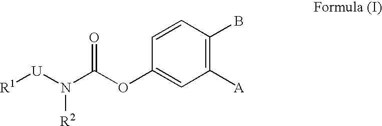

wherein:

R¹ is selected from the group consisting of:

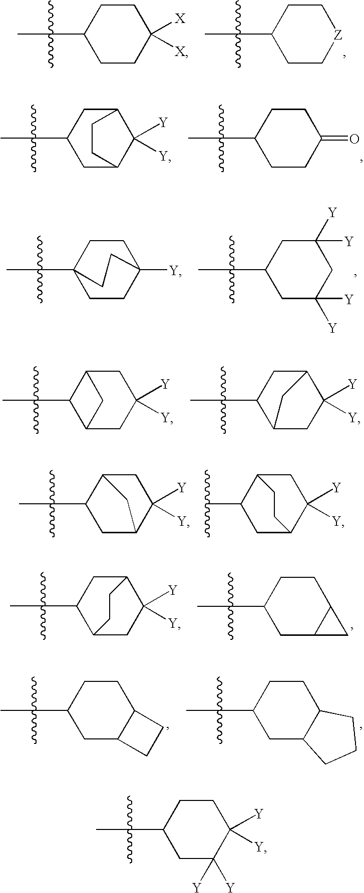

neopentyl, neohexyl, methylenecyclopropyl, methylenecyclobutyl, and methylenecyclopentyl, with the proviso that R¹ is not unsubstituted cyclohexyl;

R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂;

A and B together form an optionally substituted aromatic or non-aromatic cyclic group comprising at least one N, NR², S, or O group;

wherein each optional substituent is independently selected from C₁-C₃ alkyl, C₁-C₃ alkoxy, benzyl, halogen, nitro, cyano, or benzyloxy —C(O)R′, —C(O)-(alkyl or substituted alkyl), -(alkyl or substituted alkyl)-C(O)R′, —C(O)N(R′)₂, —C(O)N(R′)-(alkyl or substituted alkyl), -(alkyl or substituted alkyl)-C(O)N(R′)₂, —OC(O)N(R′)₂, —OC(O)N(R′)-(alkyl or substituted alkyl), -(alkyl or substituted alkyl)-OC(O)N(R′)₂, —N(R′)C(O)R′, —NR′C(O)-(alkyl or substituted alkyl), -(alkyl or substituted alkyl)-NR′C(O)R′, —SR′, —S-(alkyl or substituted alkyl), —S(O)ₖR′, where k is 1, or 2, —S(O)ₖ(alkyl or substituted alkyl), —C(S)-(alkyl or substituted alkyl), —CSN(R′)₂, —CSN(R′)-(alkyl or substituted alkyl), —N(R′)CO-(alkyl or substituted alkyl), —N(R′)C(O)OR′, -(alkyl or substituted alkyl)-O—N═C(R′)₂, -(alkyl or substituted alkyl)-C(O)NR′-(alkyl or substituted alkyl), -(alkyl or substituted alkyl)-S(O)ₖ-(alkyl or substituted alkyl)-SR′, -(alkyl or substituted alkyl)-S—SR′, —S(O)ₖN(R′)₂, —N(R′)C(O)N(R′)₂, —N(R′)C(S)N(R′)₂, —N(R′)S(O)ₖN(R′)₂, —C(R′)═NR′-C(R′)═N—N(R′)₂, and —C(R′)₂—N(R′)—N(R′)₂; and

pharmaceutically acceptable salts, pharmaceutically acceptable N-oxides, pharmaceutically active metabolites, pharmaceutically acceptable prodrugs, or pharmaceutically acceptable solvates thereof.

In one embodiment, is a compound of Formula (I) wherein R² is H. In a further embodiment, the compounds of Formula (I) are unsubstituted.

In one embodiment, is a compound of Formula (I) wherein A and B together form an optionally substituted aromatic cyclic group comprising a N and an O group. In another embodiment, is a compound of Formula (I) wherein A and B together form an optionally substituted aromatic cyclic group comprising a N and a S group. In yet a further embodiment, is a compound of Formula (I) wherein the aromatic cyclic group comprising a N and an O group is CH₃ substituted. In another embodiment, is a compound of Formula (I) wherein the aromatic cyclic group comprising a N and a S group is CH₃ substituted.

In one embodiment is a compound of Formula (IV):

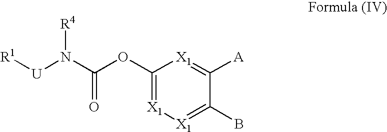

wherein:
R¹ is selected from the group consisting of:

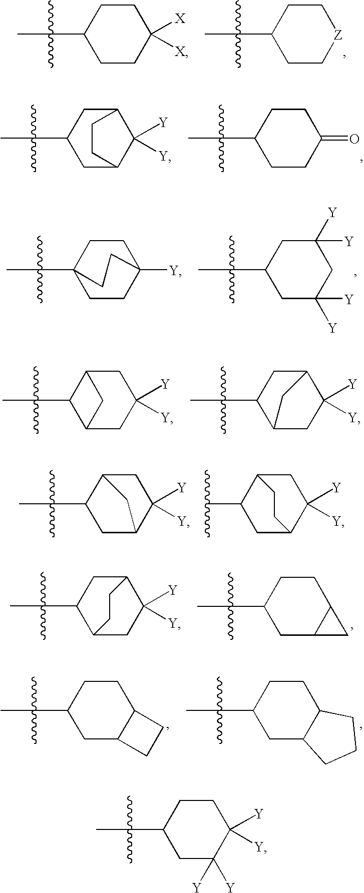

neopentyl, neohexyl, methylenecyclopropyl, methylenecyclobutyl, and methylenecyclopentyl; with the proviso that R¹ is not unsubstituted cyclohexyl;
R⁴ is H;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;

A and B together form an optionally substituted heteroaromatic group;

A and/or B are N, S, or O;

each X₁ is CH; and

pharmaceutically acceptable salts, pharmaceutically acceptable N-oxides, pharmaceutically active metabolites, pharmaceutically acceptable prodrugs, or pharmaceutically acceptable solvates thereof.

In a further embodiment is a compound selected from the group consisting of:

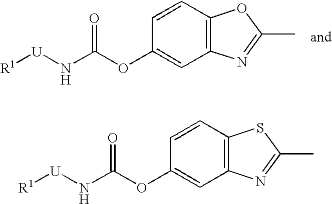

wherein:

R¹ is selected from the group consisting of:

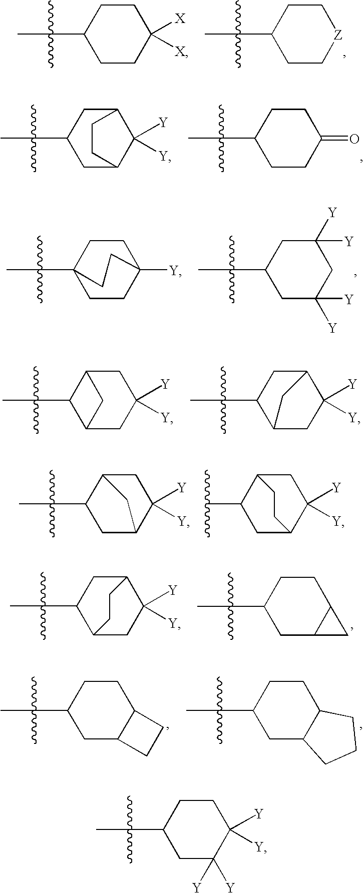

neopentyl, neohexyl, methylenecyclopropyl, methylenecyclobutyl, and methylenecyclopentyl, with the proviso that R¹ is not unsubstituted cyclohexyl;

U is a bond or CH₂; and

pharmaceutically acceptable salts, pharmaceutically acceptable N-oxides, pharmaceutically active metabolites, pharmaceutically acceptable prodrugs, or pharmaceutically acceptable solvates thereof.

In yet another embodiment is a compound of Formula (I):

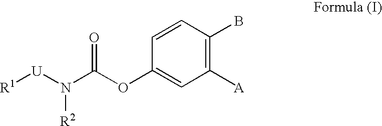

wherein:

R¹ is selected from the group consisting of:

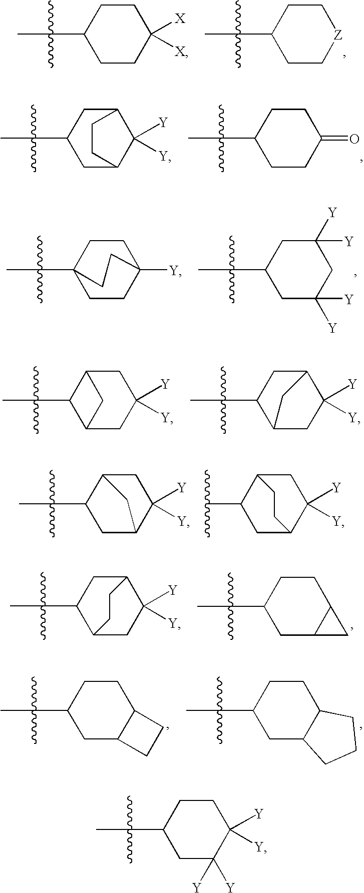

neopentyl, neohexyl, methylenecyclopropyl, methylenecyclobutyl, and methylenecyclopentyl, with the proviso that R¹ is not unsubstituted cyclohexyl;

R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂;

one of A or B is -L-G and the other is H;

L is a bond;

G is tetrazolyl; and

pharmaceutically acceptable salts, pharmaceutically acceptable N-oxides, pharmaceutically active metabolites, pharmaceutically acceptable prodrugs, or pharmaceutically acceptable solvates thereof.

In some embodiments, compounds provided herein have a structure selected from among:

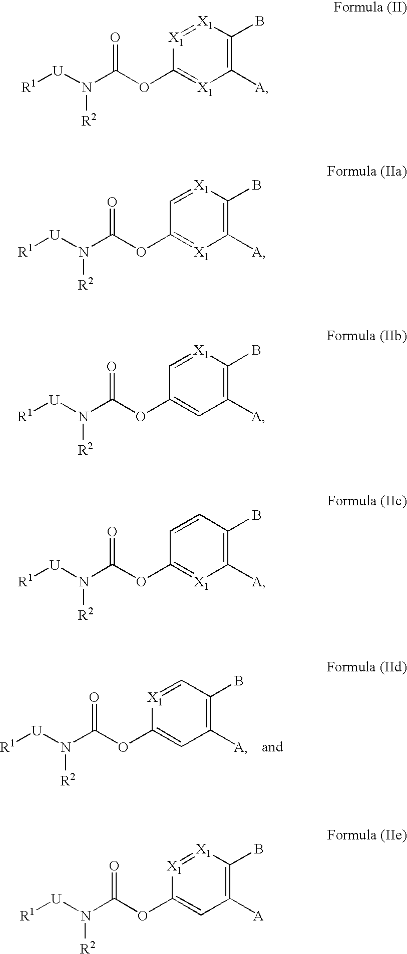

wherein:
R¹ is selected from the group consisting of:

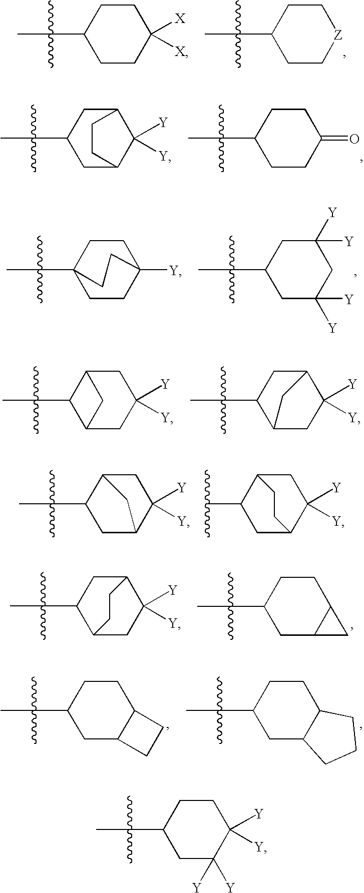

neopentyl, neohexyl, methylenecyclopropyl, methylenecyclobutyl, and methylenecyclopentyl; with the proviso that R¹ is not unsubstituted cyclohexyl;
R² is H;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;
Z is O, N—(C₁-C₆ alkyl), or SO₂;
U is a bond or CH₂;
one of A or B is -L-G and the other is H;
L is a bond;
G is tetrazolyl;
each X₁ is CH; and

pharmaceutically acceptable salts, pharmaceutically acceptable N-oxides, pharmaceutically active metabolites, pharmaceutically acceptable prodrugs, or pharmaceutically acceptable solvates thereof.

In another embodiment is a compound:

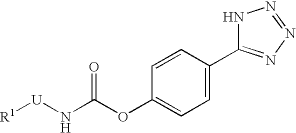

wherein:

R¹ is selected from the group consisting of:

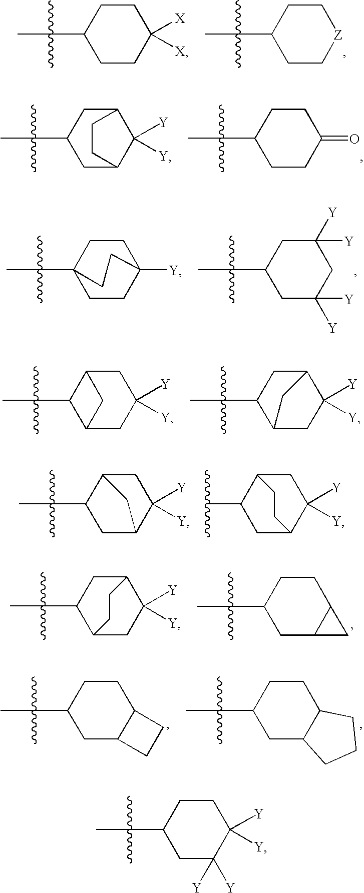

neopentyl, neohexyl, methylenecyclopropyl, methylenecyclobutyl, and methylenecyclopentyl, with the proviso that R¹ is not unsubstituted cyclohexyl;

R² is H or an optionally substituted alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group;

Z is O, N—(C₁-C₆ alkyl), or SO₂;

U is a bond or CH₂; and

pharmaceutically acceptable salts, pharmaceutically acceptable N-oxides, pharmaceutically active metabolites, pharmaceutically acceptable prodrugs, or pharmaceutically acceptable solvates thereof.

Non-limiting examples of metabolically stabilized inhibitors of fatty acid amide hydrolase, include those in Table 1.

TABLE 1

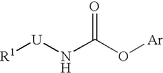

| Com- | | |
|---|---|---|
| pound | | |
| no. | R¹—U— | Ar |
| 1 | 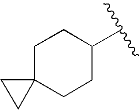 | 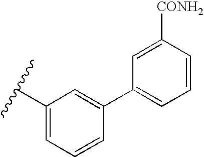 |
| 2 | 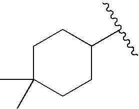 | 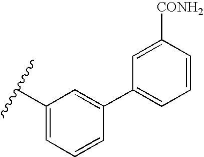 |
| 3 | 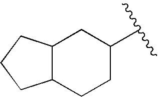 | 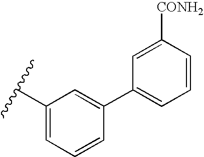 |
| 4 | 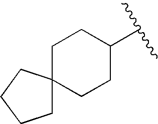 | 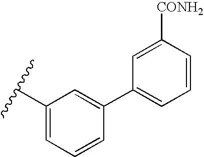 |
| 5 | 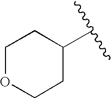 | 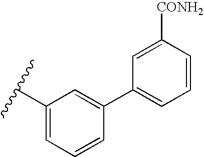 |
| 6 | 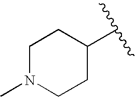 | 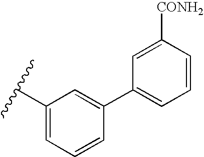 |
| 7 | 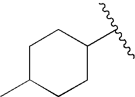 | 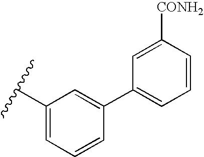 |
| 8 | 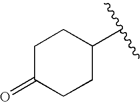 | 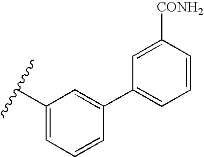 |
| 9 | 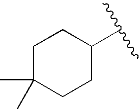 | 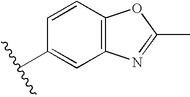 |
| 10 | 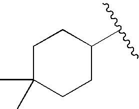 | 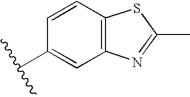 |
| 11 | 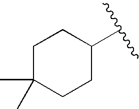 | 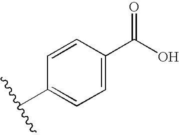 |
| 12 | 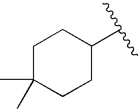 | 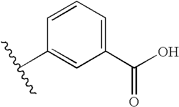 |
| 13 | 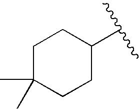 | 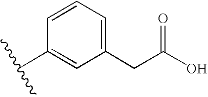 |
| 14 | 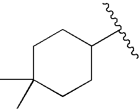 | 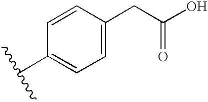 |
| 15 | 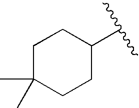 | 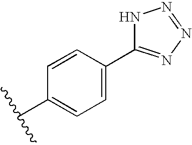 |
| 16 | 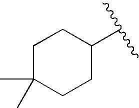 | 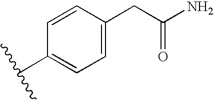 |
| 17 | 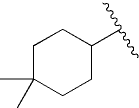 | 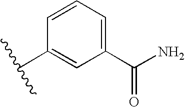 |
| 18 | 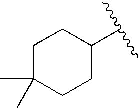 | 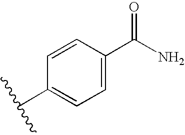 |
| 19 | 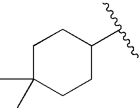 | 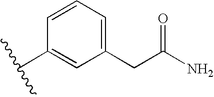 |
| 20 | 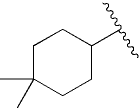 | 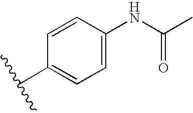 |
| 21 | 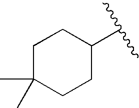 | 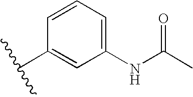 |

Any combination of the groups described above for the various variables is contemplated herein. It is understood that substituents and substitution patters on the compounds provided herein can be selected by one of ordinary skill in the art to provide compounds that are chemically stable and that can be synthesized by techniques known in the art, as well as those set forth herein.

### Preparation of Compounds

Compounds provided herein that inhibit the activity of FAAH may be synthesized using standard synthetic techniques known to those of skill in the art or using methods known in the art in combination with methods described herein. As a further guide the following synthetic methods may also be utilized.

The reactions can be employed in a linear sequence to provide the compounds described herein or they may be used to synthesize fragments which are subsequently joined by the methods described herein and/or known in the art.

### Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates. In one embodiment, a compound containing both a carboxylic acid reactive moiety and a hydroxy reactive moiety may have one of the reactive moieties blocked while the other reactive moiety is not blocked.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

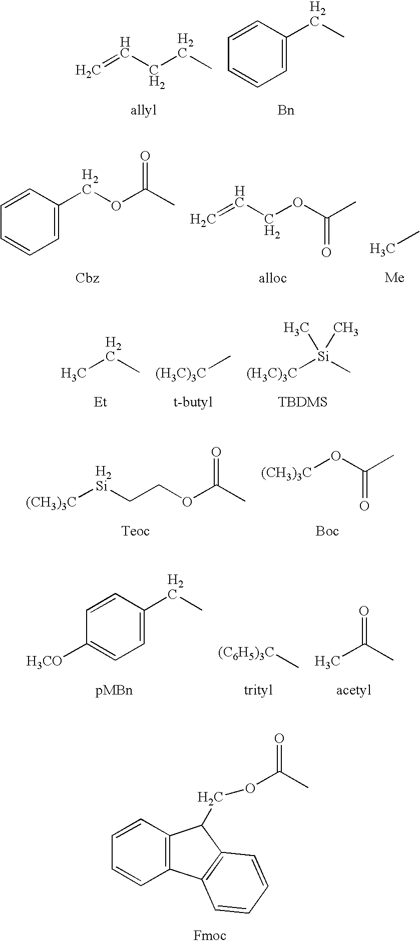

Other protecting groups are described in Greene and Wuts, Protective Groups in Organic Synthesis, 3rd Ed., John Wiley & Sons, New York, N.Y., 1999, which is incorporated herein by reference in its entirety.

### Process for the Preparation of Esters of Alkylcarbamic Acids

In certain embodiments, provided herein are methods of making and methods of using FAAH inhibitor compounds provided herein. In certain embodiments, compounds provided herein can be synthesized using the following synthetic schemes. Compounds may be synthesized using methodologies analogous to those described below by the use of appropriate alternative starting materials.

Described herein are compounds that inhibit the activity of fatty acid amide hydrolase (FAAH) and processes for their preparation. Also described herein are pharmaceutically acceptable salts, pharmaceutically acceptable N-oxides, pharmaceutically active metabolites and pharmaceutically acceptable prodrugs of such compounds. Pharmaceutical compositions that include at least one such compound or a pharmaceutically acceptable salt, pharmaceutically acceptable N-oxide, pharmaceutically active metabolite or pharmaceutically acceptable prodrug of such compound, are provided.

The synthesis of carbamates disclosed herein, such as inhibitors of fatty acid amide hydrolase described herein, may be accomplished using a variety of methods known in the art.

In one embodiment, carbamates disclosed herein are prepared by the reaction of isocyanates with hydroxy compounds, such as substituted phenols or hydroxy heteroaryls as shown in Scheme 1.

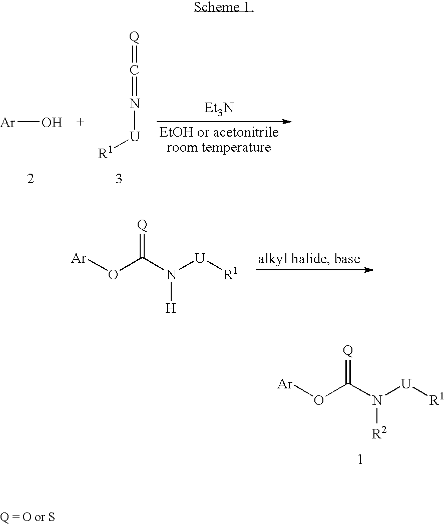

Treatment of Ar—OH (2), where Ar represents an aryl or heteroaryl, with an isocyanate or isothiocyanate (3) in the presence of a base, such as, for example, triethylamine, in an organic solvent, such as, for example, ethanol or acetonitrile, results in the formation of esters of alkylcarbamic acids of structure 1 (R²=hydrogen; see, for example, U.S. Pat. No. 5,112,859; WO 2004/033422; US 2006/0014830; J. Med. Chem. 2004, 47(21); 4998-5008; Tarzia et al. J. Med. Chem. 46:2352-2360 (2003); Kathuria et al. Nature Medicine 9(1): 76 (2003)). Isocyanates or isothiocyanates are commercially available or may be prepared by methods known in the art.

Alternatively as shown in Scheme 2, alkylcarbamic acid esters (1) may be prepared by treatment of Ar—OH (2) with alkylcarbamic acid derivatives of structure (4), where G is 4-nitrophenoxy, chlorine or imidazol-1-yl, in the presence of a base, such as, for example, triethylamine, to provide the desired compound (I).

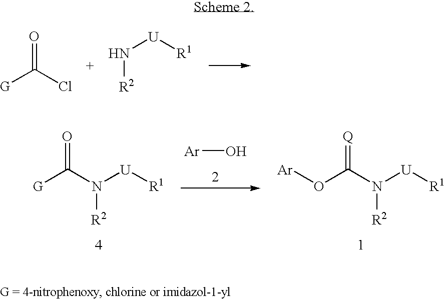

Compounds of structure (4) may be prepared using procedures well known in the art, such as, procedures described in Greene, T. W. and Wuts, P. G. M “Protective Groups in Organic Synthesis”, 3rd Edition, p. 549, New York:Wiley, 1999. Briefly, alkylamines (e.g. R¹—U—NH₂) are treated with phosgene or a phosgene equivalent, such as, for example, trichloromethyl chloroformate or carbonyldiimidazole, to yield compounds of structure (4).

Esters of alkyl(thio)carbamic acids also can be synthesized by the method outlined in Scheme 3.

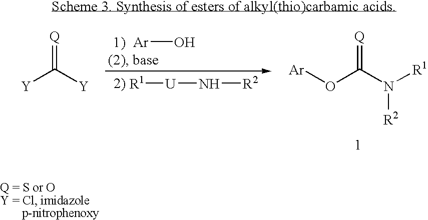

Esters of alkylcarbamic acids may also be prepared by a two-step procedure. Thiophosgene, phosgene, or an equivalent thereof (such as 4-nitrophenyl chloroformate), is first treated with Ar—OH (2) in the presence of a base in a suitable organic solvent, followed by treatment with an alkylamine such as, (R¹—U)(R²)NH. The order of the reaction can be reversed, i.e. thiophosgene, phosgene, or an equivalent thereof, can be treated with the alkylamine followed by Ar—OH (2). Equivalents of thiophosgene and phosgene include, but are not limited to, 1,1′-thiocarbonyldiimidazole, 1,1′-carbonyldiimidazole, and trichloromethyl chloroformate. Other methods for the synthesis of carbamates include those described in D. A. Black, et al. Org. Lett., 2006, 8, 1991-1993; S. Caddick, et al. Tetrahedron, 2003, 59, 5417-5423; H. Lebel, et al., Org. Lett., 2006, 8, 5717-5720; H. Lebel, et al., Org. Lett., 2005, 7, 4107-4110.

Methods for the preparation of isocyanates or isothiocyanates (3) are well known in the art. Non-limiting examples of the synthesis of isocyantes are shown in Scheme 4.

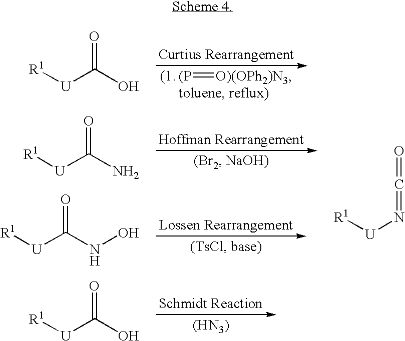

For example, isocyanates may be prepared from the corresponding carboxylic acid (i.e. R¹—U—COOH) or acid derivative (e.g. R¹—U—C(O)Cl) by treatment with an azide source such as, for example, sodium azide or diphenylphosphoryl azide followed by a Curtius-type rearrangement (see, for example, Synth. Commun. 1993, 23, 335; Heterocycles 1993, 36, 1305). In another embodiment, primary amides may be treated with bromine in the presence of a base under Hoffman conditions. The reaction of bromine with sodium hydroxide forms sodium hypobromite in situ, which transforms the primary amide into an isocyanate. In another embodiment, hydroxamic acids are treated with a dehydrating agent, such as, but not limited to tosyl chloride, under Lossen conditions. In another embodiment, carboxylic acids may be treated with HN₃ under Schmidt reaction conditions to provide isocyanates.

The requisite hydroxy-containing compounds, Ar—OH (2), can be purchased from commercial sources or prepared using procedures known in the art or outlined herein.

In one embodiment, metabolically stabilized inhibitors of fatty acid amide hydrolase may be prepared starting from commercially available ethyl 4-hydroxycyclohexanecarboxylate (SigmaAldrich, CAS Number 17159-80-7) as depicted in Scheme 5.

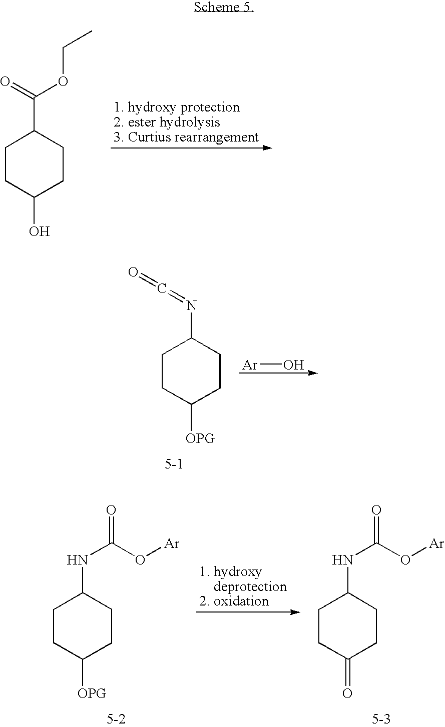

The hydroxy group of ethyl 4-hydroxycyclohexanecarboxylate is protected with a suitable protecting group, such as, for example triisopropyl silyl chloride. The ester group is hydrolyzed to provide the carboxylic acid, which is treated under Curtius rearrangement conditions to provide isocyanates of structure 5-1. Isocyanates of structure 5-1 are treated with a suitable phenol or hydroxy containing heteroaryl to furnish carbamates of structure 5-2. The protecting group of carbamate 5-2 is removed, with for example tetrabutylammonoium fluoride, and the liberated hydroxy group is oxidized to the ketone.

In another embodiment, metabolically stabilized inhibitors of fatty acid amide hydrolase may be prepared starting from aminospiranes (Rice et al. J. Med. Chem., 8, 1965, 825-829; Rice et al, J. Med. Chem. 1964, 2637; U.S. Pat. No. 3,214,470 and U.S. Pat. No. 4,005,224) as depicted in Scheme 6.

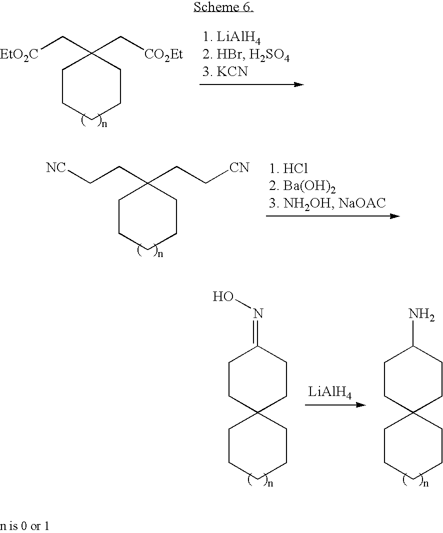

Briefly, cycloalkane-1,1-diacetic acids are obtained by the Guareschi condensation (Kon et al, J. Chem. Soc. 115, 701 (1919); Guareschi, Atti. Accad. Sci. Torino, 36, 443, (1900/1901)). Reduction of the diester to the glycols is achieved with LiAlH4 and converted into the corresponding dibromides with HBr in the presence of sulfuric acid. The dibromides are then converted into the corresponding dinitriles by treatment with KCN in aqueous alcohol. The dinitriles are then hydrolyzed to the cycloalkane-1,1-dipropionic acids, which are treated with Ba(OH)₂ to provide spiro cyclohexanones that are converted into the oxime by treatment with hydroxylamine. Reduction of the oxime with LiAlH₄ provides amines, which may then be used to prepare carbamate compounds as described herein.

In another embodiment, metabolically stabilized inhibitors of fatty acid amide hydrolase may be prepared starting from 1,4-cyclohexanediol (Sigma Aldrich, CAS Number 556-48-9) as shown in Scheme 7.

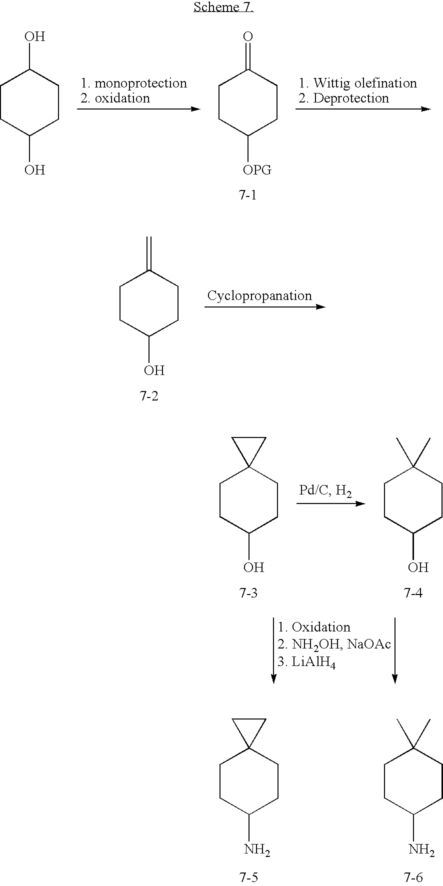

Monoprotection of 1,4-cyclohexanediol, with a suitable protecting group, such as triisopropyl silyl chloride, is followed by oxidation. Suitable oxidation conditions include, but are not limited to, Swern oxidation conditions (DMSO, Et₃N, oxalyl chloride), and tetrapropylammonium perruthenate (TPAP) with N-methylmorpholine N-oxide (NMO). Removal of the hydroxy protecting group is followed by Wittig olefination of the ketone. Cyclopropanation (e.g. Zn, CH₂I₂) of the alkene provides cyclopropane 7-3. Hydrogenation of cyclopropane 7-3 with, for example, Pd/C and hydrogen gas provides gem-dimethyl cyclohexane 7-4. Oxidation of cylcohexanol 7-3 or 7-4, provides the corresponding ketone. Treatment of the ketone with hydroxylamine provides the corresponding oximes, which are reduced, with for example LiAlH₄, to provide amines 7-5 or 7-6. Amines 7-5 or 7-6 can then be used as described above to prepare carbamates disclosed herein.

Metabolically stabilized inhibitors of fatty acid amide hydrolase may be synthesized using Diels-Alder reaction conditions as shown in schemes 8-10.

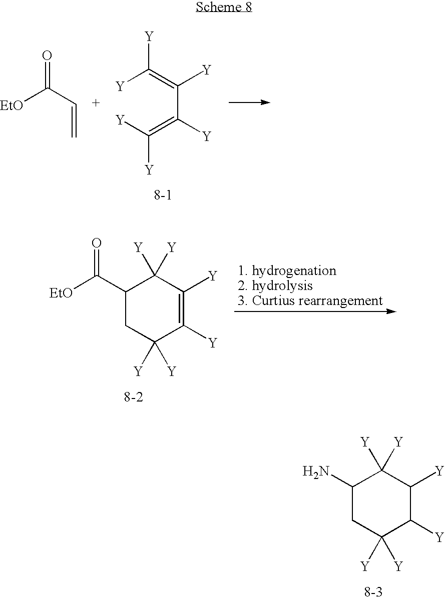

Reacting ethyl acrylate with a butadiene of structure 8-1 gives cyclohexenes of structure 8-2. Hydrogenation of the alkene with for example, Pd/C/H2 is followed by hydrolysis of the ester, and Curtius rearrangement as described above to give amines of structure 8-3. Amines of structure 8-3 are then converted to carbamates as described herein.

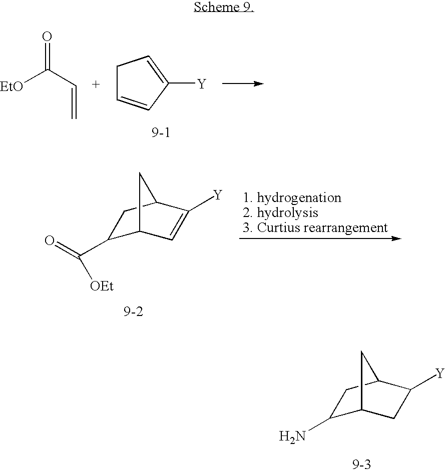

Reacting ethyl acrylate with a cyclopentadiene of structure 9-1 gives bicyclo[2.2.1]heptenes of structure 9-2. Hydrogenation of the alkene with for example, Pd/C/H₂ is followed by hydrolysis of the ester, and Curtius rearrangement as described above to give amines of structure 9-3. Amines of structure 9-3 are then converted to carbamates as described herein.

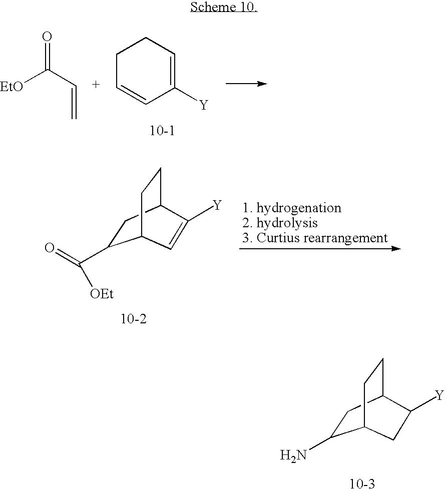

Reacting ethyl acrylate with a cyclohexadiene of structure 10-1 gives bicyclo[2.2.0]octenes of structure 10-2. Hydrogenation of the alkene with for example, Pd/C/H₂ is followed by hydrolysis of the ester, and Curtius rearrangement as described above to give amines of structure 10-3. Amines of structure 10-3 are then converted to carbamates as described herein.

Other amines that may be used to prepare metabolically stabilized inhibitors of fatty amide hydrolase as described herein include, but are not limited to: bicyclo[4.1.0]hept-3-ylamine (Avramoff., Eur. J. Med. Chem. Chim. Ther. EN; 16; 3; 1981; 199-206), bicyclo[4.2.0]oct-3-ylamine (Avramoff., Eur. J. Med. Chem. Chim. Ther. EN; 16; 3; 1981; 199-206); bicyclo[4.2.0]oct-3-ylamine (Avramoff., Eur. J. Med. Chem. Chim. Ther. EN; 16; 3; 1981; 199-206); octahydro-1H-inden-5-amine (Granger et al.; CHDCAQ; C. R. Hebd. Seances Acad. Sci. Ser. C; 265; 1967; 53); 4-aminotetrahydropyran (Apollo Scientific Ltd; CAS No. 38041-19-9); trahydrothiopyran-4-ylamine (Acros Organics; may be oxidized to the sulfone with meta-chloro peroxybenzoic acid); 4-amino-1-methylpiperidine (SynChem Inc.; CAS: 41838-46-4; CAS: 45584-07-4); 4-methyl-bicyclo-[2.2.2]-octan-1-amine; bicyclo[2.2.2]oct-1-ylamine.

The requisite hydroxy-containing compounds, Ar—OH (2), can be purchased from commercial sources or prepared using procedures known in the art or outlined herein.

Using the reaction conditions described herein, esters of alkylcarbamic acids as disclosed herein are obtained in good yields and purity. The compounds prepared by the methods disclosed herein are purified by conventional means known in the art, such as, for example, filtration, recrystallization, chromatography, distillation, and combinations thereof.

Any combination of the groups described above for the various variables is contemplated herein.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions that include a compound described herein and a pharmaceutically acceptable diluent(s), excipient(s), or carrier(s). In addition, the compounds described herein can be administered as pharmaceutical compositions in which compounds described herein are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

In certain embodiments, compositions may also include one or more pH adjusting agents or buffering agents, including organic acids such as acetic, citric, lactic, ascorbic, tartaric, maleic, malonic, fumaric, glycolic, succinic, propionic, and methane sulfonic acid; and mineral acids such as phosphoric, hydrobromic, sulfuric, boric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

A pharmaceutical composition, as used herein, refers to a mixture of a compound described herein with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

### Certain Pharmaceutical Terminology

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “acceptable” with respect to a formulation, composition or ingredient, as used herein, means having no persistent detrimental effect on the general health of the subject being treated.

As used herein, the term “selective inhibitor compound” refers to a compound that selectively inhibits a specific function/activity of one or more target proteins.

As used herein, the term “selectively inhibits” refers to the ability of a selective inhibitor compound to inhibit a specific function/activity of a target protein, such as, for example, the fatty acid amide hydrolytic activity of fatty acid amide hydrolase, with greater potency than the activity of a non-target protein. In certain embodiments, selectively inhibiting refers to inhibiting a target protein activity with a selective inhibitor that has a IC₅₀ that is at least 10, 50, 100, 250, 500, 1000 or more times lower than for that of a non-target protein activity.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “selective FAAH modulator” refers to a compound that selectively modulates at least one activity associated with FAAH.

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, 5, 10, 50, 100, 150, 200, 250, 300, 350, 400, 450 or more than 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the IC₅₀ refers to an amount, concentration or dosage of a particular test compound that achieves a 50% inhibition of a maximal response, such as inhibition of FAAH, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound administered, age, weight, general condition of the subject, the condition being treated, the severity of the condition being treated, and the judgment of the prescribing physician.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401).

By “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts also may be obtained by reacting a compound described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

“Bioavailability” refers to the percentage of the weight of compounds disclosed herein dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein are absorbed into the general circulation when the pharmaceutical composition is taken orally as compared to intravenous injection.

“Blood plasma concentration” refers to the concentration of compounds provided herein in the plasma component of blood of a subject. It is understood that the plasma concentration of compounds provided herein may vary significantly between subjects, due to variability with respect to metabolism and/or possible interactions with other therapeutic agents. In accordance with one embodiment disclosed herein, the blood plasma concentration of the compounds provided herein may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound provided herein may vary from subject to subject.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Steady state,” as used herein, is when the amount of drug administered is equal to the amount of drug eliminated within one dosing interval resulting in a plateau or constant plasma drug exposure.

### Dosage Forms

The compositions described herein can be formulated for administration to a subject via any conventional means including, but not limited to, oral, parenteral (e.g., intravenous, subcutaneous, intrathecal, or intramuscular), buccal, intranasal, epidural, pulmonary, local, rectal or transdermal administration routes. As used herein, the term “subject” is used to mean an animal, preferably a mammal, including a human or non-human. The terms patient and subject may be used interchangeably.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound provided herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof, as described in the standard reference Gennaro, A. R. et al., Remington: The Science and Practice of Pharmacy (20th Edition, Lippincott Williams & Wilkins, 2000, see especially Part 5: Pharmaceutical Manufacturing).

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound disclosed herein, the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

### Methods of Dosing and Treatment Regimens

The compounds described herein can be used in the preparation of medicaments for the inhibition of fatty acid amide hydrolase, or for the treatment of diseases or conditions that would benefit, at least in part, from inhibition of fatty acid amide hydrolase. In addition, a method for treating any of the diseases or conditions described herein in a subject in need of such treatment, involves administration of pharmaceutical compositions containing at least one compound disclosed herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to the subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve; upon the doctor's discretion the administration of the compounds may be administered chronically, that is, for an extended period of time, including throughout the duration of the patient's life in order to ameliorate or otherwise control or limit the symptoms of the patient's disease or condition.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, the daily dosages appropriate for the compounds described herein to alleviate the symptoms described herein are from about 0.001 to about 50 mg/kg per body weight. In other embodiments, the daily dosages appropriate for the compounds described herein are from about 0.01 to about 20 mg/kg per body weight. In further embodiments, the daily dosages appropriate for the compounds described herein described herein are from about 0.01 to about 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

### Combination Treatments

The compositions and methods described herein may also be used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

### EXAMPLES

The person skilled in the art may further appreciate various aspects and advantages of the present disclosure upon review of the following illustrative and non-limiting examples:

### Example 1

### General Procedure for the Preparation of Metabolically-Stabilized Inhibitors of FAAH

To a stirred solution of 4-Dimethylcyclohexylamine (1 mmol, 127 mgs) in THF (10 mL) at room temperature was added 4-nitrophenol carbonate (1 mmol, 304 mgs). After 30 minutes, 60% sodium hydride in mineral oil (1 mmol, 40 mgs) was added in one portion followed by 5′-hydroxybiphenyl-3-carboxamide (1 mmol, 213 mgs). The reaction mixture was stirred for 5 minutes and 60% sodium hydride in mineral oil (1 mmol, 40 mgs) was added in one portion. The reaction mixture was stirred for 3 more hours and was quenched with water. The crude product was extracted with ethyl acetate and the organic layer was evaporated. The residual solid was purified by reverse phase HPLC to yield the product as a white powder. MS (ESI) MH⁺:367.

### Example 2

### Methods of Screening Compounds for Metabolic Stability

Generally, a FAAH inhibitor was incubated in human liver S9 fractions. Incubations were conducted at 37° C. in a potassium phosphate buffer (pH 7.2). NADPH and a regenerating system consisting of NADP, glucose 6-phosphate dehydrogenase were provided to the incubates. Incubations were terminated by the addition of methanol and freezing at −80° C. See, e.g., Singh, R. et al. Rapid Commun. Mass Spectrom., 10: 1019-26 (1996).

### Example 3

### Methods of Screening Compound for FAAH Inhibitory Activity

Generally, a FAAH inhibitor used in the methods described herein is identified as an inhibitor of FAAH in vitro. Preferred in vitro assays detect a decrease in the level of a FAAH substrate (e.g., anandamide, OEA) or an increase in the release of a reaction product (e.g., fatty acid amide or ethanolamine) by FAAH-mediated hydrolysis of a substrate such as AEA or OEA. The substrate may be labeled to facilitate detection of the released reaction products. High throughput assays for the presence, absence, or quantification of particular reaction products are well known to those of ordinary skill in the art. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification of a large number of in vitro FAAH inhibitors without undue effort.

Candidate in vivo FAAH inhibitors can be identified by their ability to increase systemic levels of one or more FAAs. Suitable FAAs include fatty acid ethanolamides with a fatty acid moiety containing 14 to 28 carbons, with 0 to 6 double bonds, such as, for example, OEA, PEA, AEA, and stearoylethanolamide (SEA). Other suitable FAAs include primary fatty acid amides with a fatty acid moiety containing 14 to 28 carbons, with 0 to 6 double bonds, such as oleamide. Biological samples from which FAA levels can be assayed include, but are not limited to, plasma, serum, blood, cerebrospinal fluid, saliva, or urine.

FAA levels in a biological sample are assayed, e.g., by liquid chromatography tandem-mass spectrometry (LC-MS/MS). Increased assay reproducibility is achieved by spiking biological samples with a known amount of an isotopically labeled FAA, which serves as an internal standard for the FAA to be assayed. The level of the FAA can also be determined using spectrophotometric techniques (e.g., a fluorometric method). Alternatively, the level of the FAA can be determined using a biological assay. In some embodiments, the level of the FAA is determined using a combination of the aforementioned techniques. Any of the foregoing assays for FAA levels can be partly or fully automated for high throughput. Details of this and other FAA assays, as well as methods for analyzing changes in FAA levels are known in the art. See, e.g., Quistad et al. (2002), Toxicology and Applied Pharmacology 179: 57-63; Quistad et al. (2001), Toxicology and Applied Pharmacology 173, 48-55; Boger et al. (2000), Proc. Natl. Acad. Sci. U.S.A. 97, 5044-49; Cravatt et al. Proc. Natl. Acad. Sci. U.S.A. 98, 9371-9376 (2001); Ramarao et al. (2005) Anal. Biochem. 343: 143-51. See also U.S. Pat. No. 6,096,784, U.S. Pat. Publication No. 2004/0127518, U.S. patent application Ser. No. 10/681,858, International Patent Publication No. WO 98/24396, and WO 04/033422.

### Example 4

### Compound Screening for Inhibition of FAAH Activity

### FAAH LC-MS/MS Screening Assay

In one embodiment, inhibition of FAAH activity is determined using LC-MS/MS. The following are combined in a 5-mL glass tube: anandamide (5 μL of 200 ug/mL), 960 μL of 50 mM ammonium phosphate buffer (pH 7.4) containing 0.125% BSA (w/v), 10 μL of DMSO without (control) or with a FAAH inhibitor (1 μg/mL), and 25 μL of human liver microsomes (31.3/g). Prior to incubation, a 100 μL aliquot is transferred to a 96-well plate containing 0.25 mL of acetonitrile and D₄ (deuterated) anandamide (0.2 #M). Each 5-mL tube is capped and placed in a shaking water bath maintained at 37° C. for 60 minutes. After a 60 minute incubation, a second 100 μL aliquot is transferred to a 96-well plate as performed earlier. The 96-well plate is then capped, vortex mixed, and placed on an HPLC for liquid chromatography/tandem mass spectrometry (LC/MS/MS) analyses. HPLC is carried out on a Waters 2790 Alliance system (Milford, Mass.). Separation was performed on a Phenomenex C18 column (2 mm×50 mm, 4μ; Torrance, Calif.) using an isocratic mobile phase of acetonitrile:water:formic acid (80:20:0.1, v/v/v) at a flow rate of 0.3 mL min⁻¹ and a column temperature of 45° C. The HPLC system was interfaced with a Micromass Ultima tandem MS (Beverly, Mass.). The samples are analyzed using an electrospray probe in the positive ionization mode with the cone voltage set at 40 V and capillary at 3.2 kV. The source and desolvation temperature settings are 130° C. and 500° C., respectively. The voltage of the CID chamber is set at −20 eV. Multiple reaction monitoring is used for the detection of anandamide as [M+H] (m/z 348>62) and D₄ anandamide (internal standard) as [M+H] (m/z 352>66). An area ratio response (anandamide area response/D₄ anandamide area response) was determined for each sample. Percent anandamide hydrolysis of each sample is determined by the following equation, [(T=0 response)−(T=60 response)/T=0 response]*100. The percent hydrolysis normalized to control is determined by dividing the % hydrolysis of test sample by the % hydrolysis of the control sample. As an example, the compound prepared by the method of Example 1 [(4,4-dimethylcyclohexyl)carbamic acid 3′carbamoylbiphenyl-3-yl ester] at a concentration of 30 nM significantly inhibited anandamide hydrolysis in this assay, resulting in <50% anandamide hydrolysis compared to the control, 3′-carbamoylbiphenyl-3-yl cyclohexylcarbamate, which resulted in a 30% anandamide hydrolysis.

For determining IC₅₀ values for candidate FAAH inhibitor compounds, the above method is used with an adjusted FAAH inhibitor concentration. In the IC₅₀ assay, the FAAH inhibitor is added at a concentration range of approximately 3 μM to 0.03 nM. The final calculation of IC₅₀ is determined by first transforming the concentrations by “X=log(X)” and then analyzing the data with a sigmoidal dose-response curve (no constraints) using GraphPad Prism version 4.00 for Windows (GraphPad Software, San Diego Calif. USA, www.graphpad.com).

### Example 5

### Compound Screening for Inhibition of FAAH Activity

### FAAH Fluorescent Screening Assay

To a black 96-well plate (Nunc, cat #267342) is added 180 μL of arachidonyl 7-amino,4-methylcoumarin amide (AAMCA, 3 μM), 20 μL of a FAAH inhibitor (0.05 μg/mL in DMSO) and 50 μL of human liver microsomes (0.25 mg/mL). The diluent for the AAMCA and human liver microsomes is fatty acid free BSA (1.4 mg/mL) in HEPES/EDTA (50 mM/1 mM) at pH 7.4. The plate is read at excitation 355 nm and emission 460 nm at T=0 on a fluorescence plate reader (SpectraMax GeminiXS, Molecular Devices) and incubated for 30 minutes at 37° C. After the minute incubation, the plate is read a final time and % hydrolysis (normalized to control) was determined. The calculation for % hydrolysis is [(T=0−T=30)/T=0]*100. The percent hydrolysis normalized to control is determined by dividing the % hydrolysis of test sample by the % hydrolysis of the control sample (DMSO).

### Example 6

### Compound Screening for Inhibition of FAAH Activity

### Screening for In Vivo FAAH Inhibition in Rats

Potential FAAH inhibitors are formulated for oral (p.o.), intraperitoneal (i.p.) or intravenous (i.v.) delivery to rats. Formulated compounds are administered and the animals were sacrificed at pre-determined time points post dose. At sacrifice, blood samples are collected into EDTA plasma tubes and whole brains were snap frozen in liquid nitrogen. EDTA plasma was isolated from blood samples after centrifugation. Brain and plasma samples are stored at −80° C. prior to analysis. All samples (brain and plasma) are analyzed for the concentrations of test compound (FAAH inhibitor), metabolites of the test compound and endogenous fatty acid ethanolamide levels (including anandamide, oleoylethanolamide, and palmitoylethanolamide) by LC-MS/MS. Levels of these compounds are compared across time points to determine pharmacokinetic properties of the test compounds and partial pharmacological effects of inhibiting FAAH activity (including changes of fatty acid ethanolamide levels).

In one embodiment, additional tissues and fluid samples can be collected at sacrifice. In one embodiment, FAAH activity can also be determined in fluid and tissues samples according to the methods disclosed or according to methods known in the art. In one embodiment, metabolites of the test compounds can be determined in fluid and tissue samples.

### Example 7

### Determination of Pharmacokinetics

The pharmacokinetic properties of compounds provided herein were assessed in rats following oral administration as a solution. To test the oral bioavailability of compounds provided herein, a solution of the test compound was prepared for oral administration as a 10 mg/mL solutions in 80% cremephor and 20% ethanol (w/w) or as a 10 mg/mL solution of 90% PEG-400 and 10% Tween 80 (w/w). The solution of the test compound was administered to rats at a dose of 10 mg/kg via oral gavage.

### Animal Models

Any of a variety of animal models can be used to test the compounds disclosed herein for their effectiveness in reducing inflammation and treating pain. Useful compounds can exhibit effectiveness in reducing inflammation or pain in one or more animal models.

### Animal Models for Assessing Anti-inflammatory Activity

### Example 8

### Carrageenan-Induced Foot Pad Edema Model

The model is described, for example, by Winter et al. (1962 Proc Soc Exp Biol Med 111:544). Briefly, rats are fasted with free access to water for 17 to 19 hours before oral treatment with up to three doses of a test compound, indomethacin or celecoxib, or a control vehicle (1% methylcellulose in deionized water). One hour after the last treatment, paw edema is induced by injecting 0.05 ml of a 2% carrageenan solution into the left hindpaw. The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

### Example 9

### Complete Freund's Adjuvant (CFA) Induced Arthritis Model

In this model, arthritis is induced in groups of eight Lewis derived male rats weighing 160±10 g by injecting a well-ground suspension of killed Mycobacterium tuberculosis (0.3 mg in 0.1 mL of light mineral oil; Complete Freund's Adjuvant, CFA) into the subplantar region of the right hind paw on Day 1. Hind paw volumes are measured by water displacement on Days 0, 1 and 5 (right hind paw, with CFA), and on Days 0, 14 and 18 (left hind paw, without CFA); rats are weighed on Days 0 and 18. Test compounds, dissolved or suspended in 2% Tween 80, are prepared fresh daily and administered orally twice daily for 5 consecutive days (Day 1 through day 5) beginning one hour before injection of CFA. For CFA-injected vehicle control rats, the increase in paw volume on Day 5 relative to Day 1 (Acute Phase of inflammation) is generally between 0.7 and 0.9 mL; and, that on Day 18 relative to day 14 (Delayed Phase of inflammation) is generally between 0.2 and 0.4 mL. Thus, anti-inflammatory activity in this model may be denoted by values calculated during the Acute Phase as well as the Delayed Phase. Animals are also weighed on Day 0 and Day 18; CFA-injected vehicle control animals generally gain between 40 to 60 g body weight over this time period. A 30 percent or more reduction in paw volume relative to vehicle treated controls is considered of significant anti-inflammatory activity. The mean ±SEM for each treatment group is determined and a Dunnett test is applied for comparison between vehicle and treated groups. Differences are considered significant at P<0.05. Polyarthritis of fore paw, tail, nose and ear can be scored visually and noted on the first day and final day, wherein positive (+) sign is for swelling response and negative (−) sign is normal. X-ray radiographies of the hindpaws can also be performed for further radiological index determination of arthritic symptoms. Hyperalgesia can also be measured in this model, allowing determination of analgesic effects of test compounds (Bertorelli et al. 1999 Brit J. Pharmacol 128:1252).

### Example 10

### Air-Pouch Model

This model is described by Masferrer et al. (1994 Proc Natl Acad Sci USA 91:3228). Briefly, male Lewis rats (175-200 g, Harlan Sprague-Dawley) are subcutaneously injected with 20 mL of sterile air into the intrascapular area of the back to create air cavities. An additional 10 mL of air is injected into the cavity every 3 days to keep the space open. Seven days after the initial air injection, 2 mL of a 1% solution of carrageenan dissolved in sterile saline is injected directly into the pouch to produce an inflammatory response. In treated and untreated animals, the volume of exudate is measured and the number of leukocytes present in the exudate is determined by Wright-Giemsa staining. In addition, PGE₂ and 6-keto-PGF_{1α} are determined in the pouch exudates from treated and untreated animals by specific ELISAs (Cayman Chemicals, Ann Arbor, Mich.).

### Animal Models for Assessing Analgesic Activity

### Example 11

### Carrageenan-Induced Thermal Hyperalgesia

This model is described by Hargreaves et al. (1988 Pain 32:77). Briefly, inflammation is induced by subplantar injection of a 2% carrageenan suspension (0.1 mL) into the right hindpaw. Three hours later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 hour after the oral treatment with the test compound or a control. The results can be expressed as the nociceptive threshold in seconds (sec) for each hindpaw and the percentage of variation of the nociceptive threshold (mean ±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

### Example 12

### Phenylbenzoquinone-Induced Writhing Model

This model is described by Siegmund et al. (1957 Proc Soc Exp Bio Med 95:729). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5th to the 10th minute after PBQ injection, and can also be counted between the 35th and 40th minute and between the 60th and 65th minute to provide a kinetic assessment. The results are expressed as the number of stretches and writhings (mean ±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

### Example 13

### Kaolin-Induced Arthritis Model

This model is described by Hertz et al. (1980 Arzneim Forsch 30:1549). Briefly, arthritis is induced by injection of 0.1 mL of kaolin suspension into the knee joint of the right hind leg of a rat. Test compounds are administered subcutaneously after 15 minutes and again after two hours. Reference compounds can be administered orally or subcutaneously. Gait is assessed every hour from 1.5 hours to 5.5 hours after treatment and is scored as follows: normal gait (O), mild disability (1), intermittent raising of paw (2), and elevated paw (3). Results are expressed as the mean gait score (mean ±SEM) calculated from individual values at each time point and the percentage of variation of the mean score calculated from the mean value of the vehicle-treated group at 4.5 hours and 5.5 hours after treatment. The statistical significance of differences between the treated groups and the vehicle-treated group is determined by a Dunnett's test using the residual variance after a one-way analysis of variance (P<0.05) at each time point.

### Example 14

### Peripheral Mononeuropathy Model

This model is described by Bennett et al. (1988 Pain 33:87) and can be used to assess anti-hyperalgesic effect of an orally administered test compound in a model of peripheral mononeuropathy. The effect of the test substance can be compared to a no treatment control or reference substance, e.g., morphine. Peripheral mononeuropathy is be induced by loose ligation of the sciatic nerve in anaesthetized male Sprague Dawley rats (pentobarbital; 45 mg/kg by intraperitoneal route). Fourteen days later, the nociceptive threshold is evaluated using a mechanical nociceptive stimulation (analgesimeter paw pressure test; Ugo Basile, Italy). The test and reference compounds and the vehicle are orally administered (10 mL/kg carried 1% methylcellulose). Increasing pressure is applied to the hindpaw of the animal until the nociceptive reaction (vocalization or paw withdrawal) is reached. The pain threshold (grams of contact pressure) is measured in ipsilateral (injured) and in contralateral (non injured) hindpaws, 60 minutes after treatment. The results are expressed as: the nociceptive threshold (mean ±SEM) in grams of contact pressure for the injured paw and for the non-injured paw (vehicle-treated group) and the percentage of variation the nociceptive threshold calculated from the mean value of the vehicle-treated group. A comparison of the nociceptive threshold between the non injured paw and the injured paw of the vehicle-treated group is performed using a Student's t test. The statistical significance of the difference between the treated groups and the vehicle group is determined for the injured hindpaw by a Dunnett's test using the residual variance after a one-way analysis of variance (P<0.05) using SigmaStat Software (SigmaStat.RTM. v. 2.0.3 (SPSS Science Software, Erkrath GmbH)).

### Example 15

### Chung Rat Model of Peripheral Neuropathy

In one embodiment, the effectiveness of a compound provided herein in alleviating neuropathic pain is demonstrated using the well-recognized Chung rat model of peripheral neuropathy. In the Chung rat model, spinal nerve partial ligation of left spinal nerves L-5 and L-6 produces a long-lasting hypersensitivity to light pressure on the affected left foot. The hypersensitivity is similar to the pain experienced by humans with the neuropathic condition of causalgia (Kim and Chung, Pain 50:355-363 (1992), which is incorporated herein by reference).

### Example 16

### Diabetic Neuropathy Paw Pressure Test

Complete protocol details can be found in Rakieten et al. (1963 Cancer Chemother Rep 29:91). Briefly, diabetes is induced by intraperitoneal injection of streptozotocin in rats. Three weeks later, the nociceptive threshold is measured using the paw pressure test to assess hyperalgesia. Test compound or controls are administered intraperitoneally 30 minutes prior to pain measurement.

### Example 17

### Acetic Acid Writhing Test

Briefly, a test compound is administered orally one hour before intraperitoneal injection of acetic acid (0.5%, 10 ml/kg) in rats. Reduction in the number of writhes by 50 percent or more (≧50) per group of animals observed during the 5 to 11 minute period after acetic acid administration, relative to a vehicle treated control group, indicates possible analgesic activity. This assay is based on that described in Inoue, K. et al. (1991 Arzneim. Forsch./Drug Res. 41: 235).

### Example 18

### Formalin Test

Complete protocol details can be found in Hunskaar et al. (1985 Neurosci. Meth. 14:69). Briefly, 30 minutes after intraperitoneal administration of a test compound or a control, 20 μL of a 5% formalin solution is injected by subplantar route into the right hindpaw of the rat. Hindpaw licking time is recorded during the early phase and the later phase after formalin injection.

### Example 19

### Tail Flick Test

Complete protocol details can be found in D'Amour and Smith (1941 J Pharmacol. Exp Ther. 72:74). Briefly, 30 minutes after intraperitoneal administration of a test compound or a control, a light beam is focused onto the tail of the rat. The nociceptive reaction latency, characterized by tail withdrawal, is recorded. The cutoff time is set to 15 seconds.

### Example 20

### Tail Immersion Test

In this test the tail of the rat is immersed into a 50-60° C. water bath. The nociceptive reaction latency, characterized by tail withdrawal, is measured (Haubrich et al. 1990 J Pharmacol Exp Ther 255:511 and Lichtman et al. 2004 Pain 109:319).

### Example 21

### Hot Plate Test

Complete protocol details can be found in Eddy et al. (1950 J. Pharmacol. Exp. Ther. 98:121). Briefly, 30 minutes after intraperitoneal administration of a test compound or a control, the mouse is placed on a metallic hot plate maintained at 52° C. The nociceptive reaction latency, characterized by a licking reflex of the forepaws or by a jumping off the hot plate is recorded. The cut-off time is set to 30 seconds.

### Assays for Assessing Anxiolytic Activity

Compounds provided herein that inhibit FAAH activity, and thus modulate fatty acid amide levels, may also have anxiolytic activity. Animal models to assess anxiolytic activity include:

### Example 22

### Elevated Plus Maze

The elevated plus maze consists of four maze arms that originate from a central platform, effectively forming a plus sign shape as described in van Gaalen and Steckler (2000 Behavioural Brain Research 115:95). The maze can be made of plexiglas and is generally elevated. Two of the maze arms are unwalled (open) and two are walled (closed). The two open arms are well lit and the two enclosed arms are dark (Crawley 2000 What's Wrong With My Mouse?: Behavioral Phenotyping of Transgenic and Knockout Mice. Wiley-Liss, New York). The test is premised on the naturalistic conflict between the tendency of an animal to explore a novel environment and the aversive properties of a brightly lit, open area (Pellow et al. 1985 J. Neuroscience Methods. 14:149).

Complete protocol details can be found in Fedorova et al. (2001 J. Pharm. Exp. Ther. 299: 332). Briefly, 15 minutes following intraperitoneal administration of test compound or control, an animal is placed individually on the central platform, facing one of the open arms opposite to the observer. The number of open and closed arm entries, and the time spent in the different compartments of the maze by the animal (central platform, open and closed arms) is scored (as described in Gaalen et al. (supra)). An arm visit is recorded when an animal moves all four paws into the arm as described in Simonin et al. (1998 EMBO J. 17: 886). Behavior is scored by an observer and/or via a video camera over a 5-minute test session. A greater amount of time spent or entries made by the animal in the open versus the closed arms is an indicator of anxiolytic activity.

### Example 23

### Elevated Zero Maze

The elevated zero maze is a modification of the elevated plus maze. The elevated zero maze consists of a plexiglas apparatus in the shape of a circle (i.e., a circular runway of 46 cm diameter and 5.5 cm runway width) with two open and two wall-enclosed sectors of equal size. It is elevated up to a meter above the ground. This apparatus is described in Simonin et al. (supra) and Crawley (supra).

Complete protocol details can be found in Kathuria et al (2003 Nature Medicine 9: 76). Briefly, 30 minutes following intraperitoneal administration of test compound or control, an animal is placed on one open sector in front of an enclosed sector. Time in a new sector is recorded as entry with all four paws. Behavior will be scored by an observer and/or via a video camera over a 5-minute test session. A greater amount of time spent or entries made by the animal in the open versus the walled sector is an indicator of anxiolytic activity.

### Example 24

### Isolation-Induced Ultrasonic Emission Test

In another animal model, the isolation-induced ultrasonic emission test, compounds provided herein are tested for their anti-anxiety effects. The isolation-induced ultrasonic emission test measures the number of stress-induced vocalizations emitted by rat pups removed from their nest (Insel, T. R. et al., Pharmacol. Biochem. Behav., 24, 1263-1267 (1986); Miczek, K. A. et al., Psychopharmacology, 121, 38-56 (1995); Winslow, J. T. et al., Biol. Psychiatry, 15, 745-757 (1991); U.S. Pat. No. 6,326,156).

### Assays for Assessing Antinociception Mechanism

Compounds can be tested to determine if they influence pathways involved in nociception. The results of such assays can be used to investigate the mechanism by which a test compound mediates its antinociceptive effect.

### Example 25

### Elevation of 3α,5α-THP

3α-hydroxy-5α-pregan-20-one (3α,5α-THP or allopregnanolone) is a pregnane steroid that acts as an agonist of the inhibitory GABA_{A} receptor subtype and is known to have both anxiolytic and analgesic effects in a variety of animal systems, with supportive evidence for a similar role in humans. Thus, compounds that elevate 3α,5α-THP may have an antinociceptive effect. The level of 3α,5α-THP in the brain of animals treated with a test compound can be measured as described by VanDoren et al. (1982 J Neuroscience 20:200). Briefly, steroids are extracted from individual cerebral cortical hemispheres dissected in ice-cold saline after euthanasia. Cortices are frozen at −80° C. until use. Samples are digested in 0.3 N NaOH by sonication and extracted three times in 3 mL aliquots of 10% (v/v) ethyl acetate in heptane. The aliquots are combined and diluted with 4 mL of heptane. The extracts are applied to solid phase silica columns (Burdick & Jackson, Muskegon, Mich.), washed with pentane, and steroids of similar polarity to 3α,5α-THP are eluted off of the column by the addition of 25% (v/v) acetone in pentane. The eluant is then dried under N₂ and steroids are redissolved in 20% (v/v) isopropanol RIA buffer (0.1 M NaH₂PO₄, 0.9 M NaCl, 0.1% w/v BSA, pH 7.0). Extraction efficiency is determined in 50 μL of the redissolved extract by liquid scintillation spectroscopy and the remaining sample is used in the determination of 3α,5α-THP by radioimmunoassay. Reconstituted sample extracts (75 μL) and 3α,5α-THP standards (5-40,000 pg in 6.25% v/v ethanol, 31% v/v isopropyl alcohol in RIA buffer) are assayed in duplicate by the addition of 725 μL of RIA buffer, 100 μL of [³H] 3α,5α-THP (20,000 dpm), and 100 μL of anti-3α,5α-THP antibody. Total binding is determined in the absence of unlabeled 3α,5α-THP, and nonspecific binding is determined in the absence of antibody. The antibody-binding reaction is allowed to equilibrate for 120 min at room temperature and is terminated by cooling the mixture to 4° C. Bound 3α,5α-THP is separated from unbound 3α,5α-THP by incubation with 300 μL of cold dextran coated charcoal (DCC; 0.04% dextran, 0.4% powdered charcoal in double-distilled H₂O) for 20 min. DCC is removed by centrifugation at 2000×g for 10 min. Bound radioactivity in the supernatant is determined by liquid scintillation spectroscopy. Sample values are compared to a concurrently run 3α,5α-THP standard curve and corrected for extraction efficiency.

### Example 26

### Evaluation of Anti-Depressive Effects

In one embodiment, compounds provided herein are evaluated for anti-depressive effects in animal models. The chronic mild stress induced anhedonia model is based on the observation that chronic mild stress causes a gradual decrease in sensitivity to rewards, for example consumption of sucrose, and that this decrease is doses-dependent and reversed by chronic treatment with antidepressants. The method has previously been described by Willner, Paul, Psychopharmacology, 1997, 134, 319-329.

Another test for antidepressant activity is the forced swimming test (Nature 266, 730-732, 1977). In this test, animals are administered the compound preferably by the intraperitoneal route or by the oral route 30 or 60 minutes before the test. The animals are placed in a crystallizing dish filled with water and the time during which they remain immobile is clocked. The immobility time is then compared with that of the control group treated with distilled water. Imipramine (25 mg/kg) may be used as the positive control. The antidepressant compounds decrease the immobility time of the mice thus immersed.

Another test for antidepressant activity is the caudal suspension test on the mouse (Psychopharmacology, 85, 367-370, 1985). In this test, animals are preferably treated with a compound provided herein by the intraperitoneal route or by the oral route 30 minutes to 6 hours before the test. The animals are then suspended by the tail and their immobility time is automatically recorded by a computer system. The immobility times are then compared with those of a control group treated with vehicle. Imipramine (25 mg/kg) may be used as the positive control. Antidepressant compounds decrease the immobility time of the mice.

Antidepressant effects of the compounds provided herein can be tested in the DRL-72 TEST. This test, carried out according to the protocol of Andrews et al “Effects of imipramine and mirtazapine on operant performance in rats” Drug Development Research 32, 5 8-66 (1994), gives an indication of antidepressant-like activity. The effects of the compounds provided herein also may be examined in serotonin disorders and bipolar disorders, such as described in U.S. Pat. Nos. 6,403,573 and 5,952,315, incorporated herein by reference.

### Example 27

### Evaluation of Anticonvulsant Effects

In another embodiment, compounds provided herein are examined for anticonvulsant activity in animal models, as described in U.S. Pat. Nos. 6,309,406 and 6,326,156.

### Example 28

### Effects of Compounds on Appetite Behaviour

In one embodiment, compounds provided herein are administered to a rat in order to measure the effect on appetite behavior. The effect of the administered compound is assessed by examining the intake of a sucrose solution by the rat. This method is taught in W. C. Lynch et al., Physiol. Behav., 1993, 54, 877-880. Male Sprague-Dawley rats weighing about 190 g to about 210 g are under a normal light cycle (from 7 am to 7 pm) and receive water and food ad libitum. For 6 days, between 11 am and 3 pm, the food and the water bottles are withdrawn and the rats are given a 5% sucrose solution to drink. Rats drinking less than 3 g of sucrose solution are eliminated. On the seventh day the test is carried out according to the following procedure: 9 am: withdrawal of food, 10 am: administration of either a compound provided herein or vehicle to the test animals; 11 am=T0: introduction of bottles containing a weighed sucrose solution; T0+1 hour, T0+2 hours, T0+3 hours, T0+4 hours: measurement of the sucrose consumption by weighing of the bottles. Followed by comparison of the experimental (administered a compound provided herein) and control groups' intake of the sucrose solution. Animals can be, for example, obese or normal guinea pigs, rats, mice, or rabbits. Suitable rats include, for example, Zucker rats. Suitable mice include, for example, normal mice, ALS/LtJ, C3.5W-H-2b/SnJ, (NON/LtJ x NZO/H1J)Fl, NZO/H1J, ALR/LtJ, NON/LtJ, KK.Cg-AALR/LtJ, NON/LtJ, KK.CgAy/J, B6.HRS(BKS)-Cpefat/+, B6.129P2-Gcktm1Efr, B6.V-Lepob, BKS.Cg-m+1+Leprdb, and C57BL/6J with Diet Induced Obesity.

In another test, the effect of a compound of the invention on the consumption of an alcohol solution can be shown in mice. For instance, male C 57 BL 6 mice are isolated on the day of their arrival in an animal housing under a reverse cycle (night from 10 am to 10 pm) with 2 bottles filled with water. After 1 week, one of the bottles of water is replaced with a bottle filled with a 10% alcohol solution for 6 hours of the test. Each day, 30 minutes before the bottle of alcohol is introduced, the mice are treated with a compound of the invention. The amounts of alcohol and water consumed are measured after 6 hours. The test is repeated for 4 days. The results for an experimental and a control or vehicle are compared.

### Example 29

### Reduction of Body Weight, Body Fat, and Liver Steatosis

The effects of inhibiting FAAH activity on body weight, body fat, triglyceride levels, cholesterol levels can be determined in APOE*3-Leiden transgenic (E3L) mice, an animal model of hyperlipidemia. E3L mice express a mutated variant of human apoE, apoE*3-Leiden, that has impaired binding of apoE to the LDL receptor. Consequently, E3L mice exhibit a decreased clearance rate of apoB-containing lipoproteins and elevated serum lipid levels. See van Vlijmen et al. (1994), J. Clin Invest, 93:1403-1410. Upon high fat and cholesterol feeding, these mice develop various stages of atherosclerotic lesions depending on plasma total cholesterol levels and resembling those found in humans. See Groot et al. (1996), Arterioscler. Thromb. Vasc. Biol., 16:926-933; Verschuren et al. (2005), Arterioscler. Thromb. Vasc Biol., 25:161-167; and Lutgens et al. (1999), Circulation; 99(2):276-283). Thus, the E3L mouse is a suitable model for the investigation of the efficacy of anti-atherosclerotic drugs.

E3L mice are fed a high cholesterol (1% w/w) diet (HC diet) for a period of four weeks. Animals are then matched based on their plasma cholesterol levels, and are divided into five groups, each of which was maintained on an HC diet. Every day for the remainder of the study (four weeks), a “control” group receives food with no additives, a “fenofibrate” group receives food containing fenofibrate (0.04% w/w), an “oral vehicle” group receives an oral suspension of vehicle, an “oral OEA” group receives an oral suspension of OEA at a dose of 500 mg/kg, and an “oral Carbamate” group receives an oral suspension of a compound provided herein at a dose of 10 mg/kg.

Blood samples are collected at days 0, 14, and 28 of the treatment period. At the end of the treatment period, animals are sacrificed, and various tissues and organs are analyzed.

### Example 30

### Cannabinoid Receptor Binding

Compounds may exert an antinociceptive effect via binding to either or both of the cannabinoid receptors CB₁ and CB₂. CB₁ is expressed in the brain (Matsuda et al. 1990 Nature 346:561), and CB₂ is expressed by macrophages and in the spleen (Munro et al. 1993 Nature 365:61). Both of these receptors have been implicated in mediating analgesic effects through binding of agonists (see, for example, Clayton et al. 2002 Pain 96:253). Thus, test compounds can be assayed to determine whether they bind to one or both human cannabinoid receptors. An assay for CB₁ binding is described by Matsuda et al. (supra). This assay employs recombinant cells expressing CB₁. Binding to CB₂ can be determined in the same manner using recombinant cells expressing CB₂. Briefly, to measure the ability of a test compound to bind to CB₁, the binding of a labelled CB₁ ligand, e.g., [³H]WIN 55212-2 (2 nM for CB₁ and 0.8 nM for CB₂) to membranes isolated from HEK-293 cells expressing recombinant CB₁ is measured in the presence and absence of a compound. Non-specific binding is separately determined in the presence of several-fold excess of unlabelled WIN 55212-2 (5 μM for CB₁ and 10 μM for CB₂). The specific ligand binding to the receptors is defined as the difference between the total binding and the non-specific binding determined in the presence of an excess of unlabelled WIN 55212-2. The IC₅₀ values and Hill coefficients (n_{H}) are determined by non-linear regression analysis of the competition curves using Hill equation curve fitting. The inhibition constants (Kᵢ) are calculated from the Cheng Prusoff equation (Kᵢ=IC₅₀/(I+(L/K_{D})), where L=concentration of radioligand in the assay, and K_{D}=affinity of the radioligand for the receptor).

### Example 31

### Pharmaceutical Compositions

### Example 31a

### Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example 31b

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example 31c

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 31d

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 31e

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 31f

### Topical Gel Composition

To prepare a pharmaceutical topical gel composition, 100 mg of a compound described herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 31g

### Ophthalmic Solution Composition

To prepare a pharmaceutical ophthalmic composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes suggested to persons skilled in the art are to be included within the spirit and purview of this application and scope of the appended claims. All publications, patents, and patent applications cited herein are hereby incorporated by reference for all purposes.

What is claimed is:

1. A compound of Formula (I): 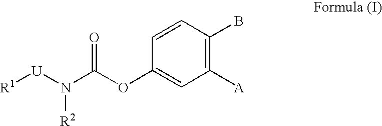
wherein:
R¹ is selected from the group consisting of: 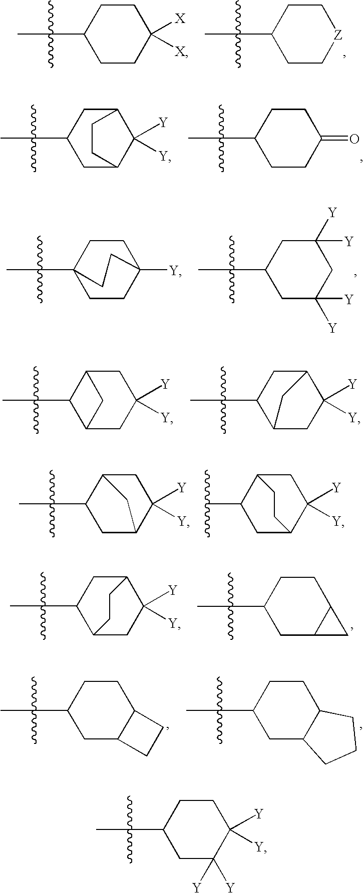
neopentyl, neohexyl, methylenecyclopropyl, methylenecyclobutyl, and methylenecyclopentyl;
each R² is independently H or C₁-C₆ saturated alkyl;
each X is independently halogen, methyl, fluoromethyl, or each X taken together can form a 3-, 4-, or 5-membered carbocyclic group;
each Y is independently H, halogen, methyl, fluoromethyl, or each Y taken together can form a 3-, 4-, or 5-membered carbocyclic group; with the proviso that R¹ is not unsubstituted cyclohexyl;
Z is O, N(C₁-C₆ saturated alkyl), or SO₂;
U is a bond or CH₂;
one of A or B is (CH₂)_{q}C(O)—C₁-C₆ alkyl, (CH₂)_{q}C(O)—N(R²)₂ and the other is H, C₁-C₆ alkyl, or C₁-C₆ heteroalkyl, q is 0, 1, 2, 3, or 4;
or A and B together form a C(O)—(CH₂)_{q}— moiety, wherein q is 1, 2, 3 or 4;
or A and B together form a 5- or 6-membered heteroaromatic group comprising at least one N, NR², S, or O group;
or A and B together form a non-aromatic or aromatic 5- or 6-membered carbocycle group;
or A and B together form an oxo-substituted 5- or 6-membered heterocyclic group comprising a heteroatom selected from N, NR², O and S;
or A and B are each independently selected from among H, C₁-C₆ alkyl, C₁-C₆ heteroalkyl, a heterocyclic group, an aryl group, a heteroaryl group, a ketoalkyl, and a ketoheteroalkyl;
or one of A or B is -L-G and the other is selected from among H and C₁-C₆ alkyl;
L is a bond, or a group selected from among C₁-C₆ alkylene, C₁-C₆ heteroalkylene, C₁-C₆ ketoalkylene, —C(O)NR⁹—(CH₂)ₙ—, —NR⁹—C(O)—(CH₂)ₙ—, —OC(O)O—(CH₂)ₙ—, —N—HC(O)O—(CH₂)ₙ—, —O(O)CNH—(CH₂)ₙ—, —C(O)O—(CH₂)ₙ—, or —OC(O)—(CH₂)ₙ—, —NR⁹C(O)N(R⁹)—(CH₂)ₙ—, —S(O)—(CH₂)ₙ—, —S(O)₂—(CH₂)ₙ—, —C(═NR¹⁰)N(R⁹)—(CH₂)ₙ—, and —NR⁹C(═NR¹⁰)N(R⁹)—(CH₂)ₙ—;
G is H, tetrazolyl, —NHS(═O)₂R⁸, —S(═O)₂NHR⁸, —S(═O)₂NH-phenyl, —OH, —SH, —OC(O)NHR⁸, —NHC(O)OR⁸, —C(O)NHC(O)R⁸, —C(O)NHS(═O)₂R⁸, —S(═O)₂NHC(O)R⁸, —S(═O)₂NHC(O)NHR⁸, —NHC(O)R⁸, —NHC(O)N(R⁹)₂, —C(═NR¹⁰)N(R⁹)₂, —NR⁹C(═NR¹⁰)N(R⁹)₂, —NR⁹C(═NR¹⁰)NHC(═NR¹⁰)N(R⁹)₂, —NR⁹C(═CHR¹⁰)N(R⁹)₂, —C(O)NR⁹C(═NR¹⁰)N(R⁹)₂, —C(O)NR⁹C(═CHR¹⁰)N(R⁹)₂, —CO₂H, —(OP(═O)OH)ₓOH, —OP(═O)OR⁸OH, —OP(═O)R⁸OH, —NR⁹P(═O)OR⁸OH, —NR⁹P(═O)R⁸OH, —P(═O)OR⁸OH; —P(═O)R⁸OH, —S(O)_{y}OH; —OS(O)_{y}OH; —NR⁹S(O)_{y}OH;
each R⁸ is independently C₁-C₆ alkyl;
each R⁹ is independently H or C₁-C₆ alkyl;
each R¹⁰ is independently selected from among H, —S(═O)₂R⁸, —S(═O)₂NH₂, —C(O)R⁸, —CN, and —NO₂;
n is 1, 2, 3, or 4; x is 1, 2, or 3; y is 1 or 2; and
pharmaceutically acceptable salts, pharmaceutically acceptable N-oxides, pharmaceutically active metabolites, pharmaceutically acceptable prodrugs, or pharmaceutically acceptable solvates thereof.

2. The compound of claim 1, wherein one of A or B is -L-G and the other is H.

3. The compound of claim 1, wherein A is -L-G.

4. The compound of claim 1, wherein B is -L-G.

5. The compound of claim 1, wherein R² is H.

6. The compound of claim 2, wherein:
L is a bond, or a group selected from among C₁-C₆ alkylene, —NR⁹—C(O)—(CH₂)ₙ—.

7. The compound of claim 6 wherein L is —NR⁹—C(O)—(CH₂)ₙ—, G is H; R⁹ is H; and n is 1.

8. The compound of claim 6, wherein L is a bond.

9. The compound of claim 8 wherein G is —CO₂H.

10. The compound of claim 6 wherein L is CH₂; and G is —CO₂H.

11. The compound of claim 8 wherein G is tetrazolyl.

12. The compound of claim 1, wherein one of A or B is (CH₂)_{q}C(O)—C₁-C₆ alkyl, (CH₂)_{q}C(O)—N(R²)₂ and the other is H, C₁-C₆ alkyl, or C₁-C₆ heteroalkyl, and q is 0, 1, 2, 3, or 4.

13. The compound of claim 12 wherein one of A or B is (CH₂)_{q}C(O)—N(R²)₂; wherein q is 0, R² is H; and the other is H.

14. The compound of claim 12 wherein one of A or B is (CH₂)_{q}C(O)—N(R²)₂; wherein q is 1, R² is H; and the other is H.

15. The compound of claim 1 wherein A and B together form a 5- or 6-membered heteroaromatic group comprising at least one N, NR², S, or O group.

16. The compound of claim 15 wherein A and B together form a 5- or 6-membered heteroaromatic group comprising N and S.

17. The compound of claim 15 wherein A and B together form a 5- or 6-membered heteroaromatic group comprising N and O.

18. The compound of claims 16 and 17 wherein the heteroaromatic group is substituted with a CH₃ group.

19. A pharmaceutical composition comprising a compound of Formula (I), pharmaceutically acceptable salt, pharmaceutically acceptable N-oxide, pharmaceutically active metabolite, pharmaceutically acceptable prodrug, or pharmaceutically acceptable solvate and a pharmaceutically acceptable diluent, excipient or binder.

20. A method of treatment comprising administering to a patient having pain a therapeutically effective amount of a compound of Formula (I), pharmaceutically acceptable salt, pharmaceutically acceptable N-oxide, pharmaceutically active metabolite, pharmaceutically acceptable prodrug, or pharmaceutically acceptable solvate.

21. The method of claim 20, wherein the pain is selected from nociceptive pain, neuropathic pain, inflammatory pain, non-inflammatory pain, painful hemorrhagic cystitis, pain associated with the herpes virus, pain associated with diabetes, peripheral neuropathic pain, peri-operative pain, cancer pain, pain and spasticity associated with multiple sclerosis, central pain, deafferentiation pain, chronic nociceptive pain, stimulus of nociceptive receptors, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, myofascial pain, and phantom and transient acute pain.

22. Use of a compound of claim 1 for inhibiting the activity of fatty acid amide hydrolase activity or for the treatment of pain in a human patient.

23. Use of a compound of claim 1 for the formulation of a medicament for the treatment of pain.

24. An article of manufacture, comprising packaging material, a compound of claim 1, which is effective for inhibiting the activity of fatty acid amide hydrolase (FAAH), within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for inhibiting the activity of fatty acid amide hydrolase (FAAH).
